(19) United States
(12) Patent Application Publication
(10) Pub. No.: US 2011/0166162 A1
(43) Pub. Date: Jul. 7, 2011

(54) 1,2-Benzisothiazole Compounds Useful for Combating Animal Pests

(75) Inventors: Wolfgang von Deyn, Neustadt (DE); Michael Puhl, Hirschberg (DE); Matthias Pohlman, Freinsheim (DE); Michael Rack, Eppelheim (DE); Liliana Parra Rapado, Offenburg (DE); Jürgen Langewald, Mannheim (DE); Douglas D. Anspaugh, Apex, NC (US); Hassan Oloumi-Sadeghi, Raleigh, NC (US); Gale F. Matt Hews, RTP, NC (US); Deborah L. Culbertson, Fuquay Varina, NC (US); Nancy B. Rankl, Cary, NC (US); Hennicke Kamp, Bischheim (DE); Bennard van Ravenzwaay, Altrip (DE)

(73) Assignee: BASF SE, Ludwigshafen (DE)

(21) Appl. No.: 12/999,349

(22) PCT Filed: Jun. 17, 2009
(86) PCT No.: PCT/EP09/57531

§ 371 Date: Mar. 11, 2011

(30) Foreign Application Priority Data
Jul. 21, 2007 (DE) 10 2007 034 158.1

(51) Int. Cl. A01N 43/54; C07D 417/06; C07D 401/06; A01N 43/40; A01P 11/00; A01P 15/00; A01P 7/04; A01P 5/00; A01P 7/00

(52) U.S. Cl. 514/256; 546/271.1; 544/333; 514/338

(57) ABSTRACT

The present invention relates to sulfonamide compounds of formula (I)

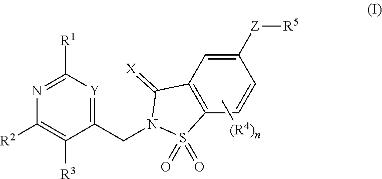

wherein R¹ is H, halogen, CN, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl or C₁-C₆-haloalkoxy; R² and R³ are H, halogen, CN, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl or C₁-C₆-haloalkoxy; or R² together with R³ form a fused 5 or 6-membered carbocycle or heterocycle; R⁴ is halogen, CN, C₁-C₆-alkyl, C₂-C₆-alkenyl, C₂-C₆-alkynyl, C₁-C₆-alkoxy, C₁-C₆-alkylthio, C₁-C₆-alkylsulfinyl, C₁-C₆-alkylsulfonyl, C₁-C₆-haloalkyl, C₁-C₆-haloalkoxy, C₁-C₆-haloalkylthio, C₁-C₆-haloalkylsulfinyl or C₁-C₆-haloalksulfonyl; n is 0, 1, 2 or 3; R⁵ is phenyl or a 5- or 6-membered heterocycle; X is O or NR^{x}, wherein R^{x} is H, C₁-C₆-alkyl, C₂-C₆-alkenyl, C₂-C₆-alkynyl, C₃-C₇-cycloalkyl, C₁-C₆-alkoxy, C₂-C₆-alkenyloxy, C₂-C₆-alkynyloxy, C₁-C₆-alkylcarbonyl or C₁-C₆-alkylcarbonyloxy; Y is N or C(R^{y}), wherein R^{y} is H, halogen, CN, C₁-C₆-alkyl, C₂-C₆-alkenyl, C₂-C₆-alkynyl, C₃-C₇-cycloalkyl, C₃₋₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy; and Z is a chemical bond, O or N(R^{z}), wherein R^{z} is C₁-C₆-alkyl, C₂-C₆-alkenyl, C₂-C₆-alkynyl, C₃-C₇-cycloalkyl, C₁-C₆-alkoxy, C₂-C₆-alkenyloxy, C₁-C₆-alkylcarbonyl or C₁-C₆-alkylcarbonyloxy;
as well as to the N-oxides and salts thereof. These compounds are useful for combating animal pests. The invention also relates to a process for the preparation of these compounds and to intermediate compounds used in said process. The invention further relates to a method for controlling animal pests by using the compounds of formula (I), the N-oxides or the salts thereof, to plant propagation material and to an agricultural and veterinary composition comprising said compounds, the N-oxides or the salts thereof.

The present invention relates to sulfonamide compounds which are useful for combating animal pests as well as to process for the preparation of those compounds and to precursor compounds used in that process. The invention further relates to a method for controlling animal pests by using these compounds, to plant propagation material and to an agricultural and veterinary composition comprising said compounds.

### BACKGROUND OF THE INVENTION

Animal pests and in particular invertebrate pests such as arthropods and nematodes, but also rodent pests, destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating animal pests. It is therefore an object of the present invention to provide compounds having a good pesticidal activity and showing a broad activity spectrum against a large number of different animal pests, especially against difficult to control arthropod pests such as insects or arachnids and nematodes and also rodent pests. Because of their special eating and social behavior, an effective rodenticide should be palatable in lethal concentrations and have a delayed toxic effect.

WO 2006/097489 describes biphenylsulfonamides of the formula (A),

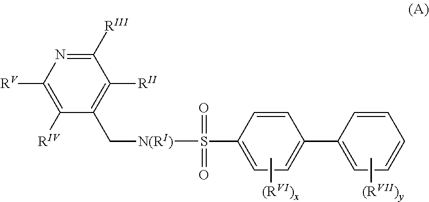

wherein R^{I} is e.g. H, alkyl, alkoxy, alkenyl, alkynyl or benzyl, R^{II} to RV are e.g. H, halogen, alkyl, halomethyl, alkoxy or halomethoxy, x is 0, 1, 2, 3 or 4, y is 0, 1, 2, 3, 4 or 5 and R^{V} and R^{VII} are e.g. H, halogen, hydroxyl, cyano, alkyl, haloalkyl, alkoxy, haloalkoxy etc.; and their use for controlling phytopathogenic harmful fungi and harmful arthropodes.

WO 2008/031824 describes biphenyl-4-ylsulfonamide compounds of formula (B)

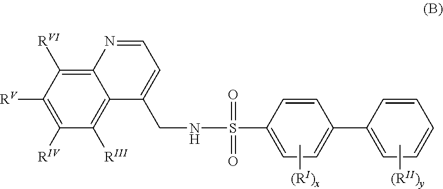

wherein x is 1 or 2, y is 0, 1, 2, 3, 4 or 5, R^{I} and RII are e.g. halogen, hydroxy, cyano, amino, nitro, alkyl, alkenyl, alkynyl, cycloalkyl, cycloalkylalkyl, alkoxy, alkenyloxy, alkynyloxy, alkoxyalkoxy, cycloalkylalkoxy, C(OH)(CF₃)₂, haloalkyl, haloalkenyl, haloalkoxy, haloalkenyloxy, alkylthio, haloalkylthio, alkylsulfinyl, haloalkylsulfinyl, alkylsulfonyl, haloalkylsulfonyl etc. and R^{III} to R^{VI} are e.g. hydrogen, halogen, hydroxy, cyano, amino, nitro, alkyl, alkoxy, haloalkyl, haloalkoxy, lkylthio, haloalkylthio, alkylsulfinyl, haloalkylsulfinyl, alkylsulfonyl, haloalkylsulfonyl etc., and their use for combating arthropod pests and nematodes.

### DESCRIPTION OF THE INVENTION

It is an object of the present invention to provide compounds that have a good pesticidal activity, in particular insecticidal activity, and show a broad activity spectrum against a large number of different animal pests, especially against difficult to control arthropod pests such as insects or arachnids.

It is another object of the present invention to provide compounds that have a good pesticidal activity against rodents and other vertebrate pests.

Surprisingly it has been found that these and further objects are achieved by sulfonamide compounds of formula (I)

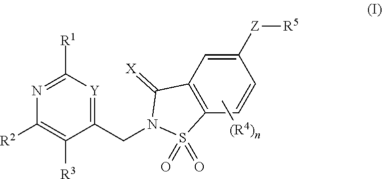

wherein
R¹ is selected from hydrogen, halogen, cyano, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy;
R² and R³ are independently selected from hydrogen, halogen, cyano, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy; or
R² together with R³ and together with the carbon atoms to which they are bound form a fused 5 or 6-membered carbocycle or a fused 5- or 6-membered heterocycle, which contains 1 or 2 heteroatoms selected from O, N and S as ring members, wherein each fused carbocycle or heterocycle is unsubstituted or carries 1, 2, 3 or 4 substituents, independently of one another selected from halogen, cyano, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy;
R⁴ is selected from halogen, cyano, C₁-C₆-alkyl, C₂-C₆-alkenyl, C₂-C₆-alkynyl, C₁-C₆-alkoxy, C₁-C₆-alkylthio, C₁-C₆-alkylsulfinyl, C₁-C₆-alkylsulfonyl, C₁-C₆-haloalkyl, C₁-C₆-haloalkoxy, C₁-C₆-haloalkylthio, C₁-C₆-haloalkylsulfinyl and C₁-C₆-haloalksulfonyl;
n is 0, 1, 2 or 3;
R⁵ is selected from phenyl and a 5- or 6-membered heterocycle Het, which contains 1, 2 or 3 heteroatoms selected from O, N and S as ring members, wherein phenyl and Het are unsubstituted or carry 1, 2, 3 or 4 substituents, independently of one another selected from halogen, cyano, C₁-C₆-alkyl, C₂-C₆-alkenyl, C₂-C₆-alkynyl, C₃-C₇-cycloalkyl, C₁-C₆-alkoxy, C₁-C₆-alkylthio, C₁-C₆-alkylsulfinyl, C₁-C₆-alkylsulfonyl, C₁-C₆-haloalkyl, C₁-C₆-haloalkoxy C₁-C₆-haloalkylthio, C₁-C₆-haloalkylsulfinyl and C₁-C₆-haloalkylsulfonyl;
X is O or NR^{x}, wherein R^{x} is selected from hydrogen, C₁-C₆-alkyl, C₂-C₆-alkenyl, C₂-C₆-alkynyl, C₃-C₇-cycloalkyl, C₁-C₆-alkoxy, C₂-C₆-alkenyloxy, C₂-C₆-alkynyloxy, C₁-C₆-alkylcarbonyl and C₁-C₆-alkylcarbonyloxy;
Y is N or C(R^{y}), wherein R^{y} is selected from hydrogen, halogen, cyano, C₁-C₆-alkyl, C₂-C₆-alkenyl, C₂-C₆-alkynyl, C₃-C₇-cycloalkyl, C₃₋₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy; and
Z is a chemical bond, O or N(R^{z}), wherein R^{z} is selected from C₁-C₆-alkyl, C₂-C₆-alkenyl, C₂-C₆-alkynyl, C₃-C₇-cycloalkyl, C₁-C₆-alkoxy, C₂-C₆-alkenyloxy, C₁-C₆-alkylcarbonyl and C₁-C₆-alkylcarbonyloxy;
   and the N-oxides and salts thereof.

Accordingly, the present invention relates to sulfonamide compounds of formula (I) and to the N-oxides and salts thereof.

The compounds according the present invention, i.e. the compounds of the formula I, their salts, the N-oxides of I and the salts of the N-oxides have a good pesticidal activity against invertebrate pests, in particular against arthropod pests and especially against insect pests, and show a broad activity spectrum against a large number of different animal pests, especially against difficult to control arthropod pests such as insects or arachnids. The compounds of the present invention also show a delayed toxic effect against certain vertebrate pests, in particular against rodents which renders the suitable for controlling rodents and other vertebrate pests.

The present invention also relates to a process for the preparation of the sulfonamide compounds of formula (I), which process comprises reacting a compound of the formula (II)

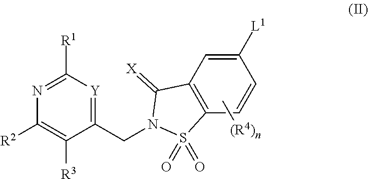

with a boronic acid derivative of the formula R⁵—(Z)—B(OR^{b1})(OR^{b2}) in the presence of a base and a transition metal catalyst to give sulfonamide compounds of formula (I), wherein R¹, R², R³, R⁴, R⁵, X, Y, Z and n are as defined for the compounds of formula (I) in any of claims 1 to 15 and wherein L¹ is a suitable leaving group and R^{b1} and R^{b2} are each independently hydrogen or C₁-C₄-alkyl, or R^{b1} and R^{b2} together form an 1,2-ethylene moiety the carbon atoms of which may be unsubstituted or may all or in part be substituted by methyl groups.

Sulfonamide compounds of formula (II),

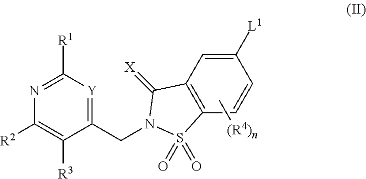

wherein R¹, R², R³, R⁴, n, X and Y have one of the meanings as defined herein for the compounds of formula (I) and wherein L¹ is halogen, are novel and useful intermediates for the preparation of the sulfonamide compounds of formula (I) according to the present invention. Accordingly, the present invention further relates to compounds of formula (II) as defined herein.

The present invention further relates to a composition, in particular an agricultural or a veterinary composition or a bait composition containing at least one sulfonamide compound of formula (I) as defined herein, an N-oxide thereof and/or a salt thereof, and at least one liquid or solid carrier.

The present invention further relates to a method for controlling animal pests which method comprises treating the pests, their food supply, their habitat or their breeding ground or a plant, seed, soil, area, material or environment in which the pests are growing or may grow, or the materials, plants, seeds, soils, surfaces or spaces to be protected from pest attack or infestation with a pesticidally effective amount of an sulfonamide compound of formula (I) or with a pesticidally effective amount of an agricultural composition containing at least one sulfonamide compound of formula (I) as defined herein.

A further aspect of the present invention relates to a method for protecting plant propagation material and/or the plants which grow therefrom, which method comprises treating the plant propagation material with a pesticidally effective amount of a compound of the formula (I) according to the present invention or an agriculturally acceptable salt or an N-oxide thereof.

A further aspect of the present invention relates to plant propagation material, comprising at least one compound of formula (I) according to the present invention and/or an agriculturally acceptable salt or an N-oxide thereof.

The present invention further relates to a method for treating or protecting an animal, e.g. mammals, birds or fish, from infestation or infection by parasites which comprises bringing the animal in contact with a parasiticidally effective amount of a sulfonamide compound of formula (I) or a veterinarily acceptable salt or an N-oxide thereof as defined herein. Bringing the animal in contact with the compound of the formula (I), its salt or the veterinary composition of the invention means applying or administering it to the animal.

The present invention further relates to the use of sulfonamide compounds of formula (I) or the veterinarily acceptable salts or the N-oxides thereof as defined herein for controlling animal pests.

### DETAILED DESCRIPTION OF THE INVENTION

The radicals attached to the backbone of the compounds of formula (I) may contain one or more centers of chirality. In this case the compounds of the formula (I) are present in the form of different enantiomers or diastereomers, depending on the substituents. The present invention relates to every possible stereoisomer of the compounds of formula (I) i.e. to single enantiomers or diastereomers, as well as to mixtures thereof.

The compounds of the present invention may be amorphous or may exist in one or more different crystalline states (polymorphs) which may have a different macroscopic properties such as stability or show different biological properties such as activities. The present invention includes both amorphous and crystalline compounds of the formula I, mixtures of different crystalline states of the respective compound I, as well as amorphous or crystalline salts thereof.

The term “animal pest” as used herein encompasses animal populations, in particular populations of invertebrate pests, such as insects, arachnids and nematodes. These pests may attack plants thereby causing substantial damage to the plants attacked. The term “animal pest” as used herein encompasses ectoparasites which may infest animals, e.g. mammals, birds or fish, thereby causing substantial damage to the animals infested. The term “animal pest” as used herein also encompasses animal populations of rodents may attack plants or plant propagation material thereby causing substantial damage to the plants attacked or which may be carrier of diseases.

Agriculturally useful salts of the compounds (I) encompass especially the salts of those cations or the acid addition salts of those acids whose cations and anions, respectively, have no adverse effect on the pesticidal action of the compounds (I). Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl, hydroxy-C₁-C₄-alkyl, C₁-C₄-alkoxy-C₁-C₄-alkyl or hydroxy-C₁-C₄-alkoxy-C₁-C₄-alkyl substituents and/or one phenyl or benzyl substituent, examples including, ammonium, methylammonium, isopropylammonium, diisopropylammonium, tetramethylammonium, tetraethylammonium, tetrabutylammonium, 2-hydroxyethylammonium, 2-(2-hydroxyethoxy)eth-1-ylammonium (diglycolamine salts), di(2-hydroxyeth-1-yl)ammonium (diolamine salts), tris((2-hydroxyeth-1-yl)ammonium (trolamine salts), tris(3-propanol)ammonium, trimethylbenzylammonium, triethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting compounds of formula (I) with an acid of the corresponding anion, preferably of hydrochloric acid, hydrobromic acid, sulfuric acid, phosphoric acid or nitric acid.

Veterinarily acceptable salts of the compounds of formula (I) encompass especially the salts of those cations or the acid addition salts which are known and accepted in the art for the formation of salts for veterinary use. Suitable acid addition salts, e.g. formed by compounds of formula (I) containing a basic nitrogen atom, e.g. an amino group, include salts with inorganic acids, for example hydrochlorids, sulphates, phosphates, and nitrates and salts of organic acids for example acetic acid, maleic acid, e.g. the monoacid salts or diacid salts of maleic acid, dimaleic acid, fumaric acid, e.g. the monoacid salts or diacid salts of fumaric acid, difumaric acid, methane sulfenic acid, methane sulfonic acid, and succinic acid.

In the definitions of the variables given above, collective terms are used which are generally representative for the substituents in question. The term Cₓ-C_{y} indicates the number of carbon atoms possible in each case in the substituent or substituent moiety in question.

The term “halogen” as used herein refers to fluoro, chloro, bromo and iodo.

The term “C₁-C₆-alkyl” as used herein and in the alkyl moieties of C₁-C₆-alkoxy, C₁-C₆-alkylcarbonyl, C₁-C₆-alkylcarbonyloxy, C₁-C₆-alkylsulfinyl, C₁-C₆-alkylsulfonyl, C₁-C₆-alkylthio refers to a straight-chained or branched saturated hydrocarbon group having 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and the like.

Accordingly, “C₁-C₆-alkoxy” and “C₁-C₆-alkylthio” as used herein refer to straight-chain or branched alkyl groups having 1 to 6 carbon atoms (as mentioned above) bonded through an oxygen atom or a sulfur atom respectively, at any position in the alkyl group. Examples include methoxy, ethoxy, propoxy, isopropoxy, methylthio, ethylthio, propylthio, isopropylthio, n-butylthio and the like.

Accordingly, “C₁-C₆-alkylsulfinyl” and “C₁-C₆-alkylsulfonyl” refer to straight-chain or branched alkyl groups having 1 to 6 carbon atoms (as mentioned above) bonded through a—S(═O)— moiety or a —S(═O)₂— moiety, respectively, at any position in the alkyl group. Examples include methylsulfinyl, methylsulfonyl and the like.

Accordingly, “C₁-C₆-alkylcarbonyl” and “C₁-C₆-alkylcarbonyloxy” refer to straight-chain or branched alkyl groups having 1 to 6 carbon atoms (as mentioned above) bonded through a —C(═O)— moiety or a —C(═O)O— moiety, respectively, at any position in the alkyl group. Examples include acetyl, acetyloxy and the like.

The term “C₁-C₆-haloalkyl” as used herein and in the haloalkyl moieties of C₁-C₆-haloalkoxy, C₁-C₆-haloalkylthio, C₁-C₆-haloalkylsulfinyl and C₁-C₆-haloalkylsulfonyl, refers to a straight-chained or branched alkyl group having 1 to 6 carbon atoms (as mentioned above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 5-fluoro-1-pentyl, 5-chloro-1-pentyl, 5-bromo-1-pentyl, 5-iodo-1-pentyl, 5,5,5-trichloro-1-pentyl, undecafluoropentyl, 6-fluoro-1-hexyl, 6-chloro-1-hexyl, 6-bromo-1-hexyl, 6-iodo-1-hexyl, 6,6,6-trichloro-1-hexyl, dodecafluorohexyl and the like.

Accordingly, “C₁-C₆-haloalkoxy” and “C₁-C₆-haloalkylthio” as used herein refer to straight-chain or branched haloalkyl groups having 1 to 6 carbon atoms (as mentioned above) bonded through an oxygen atom or a sulfur atom respectively, at any position in the alkyl group. Examples include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio and the like.

Accordingly, “C₁-C₆-haloalkylsulfinyl” and “C₁-C₆-haloalkylsulfonyl” refer to straight-chain or branched haloalkyl groups having 1 to 6 carbon atoms (as mentioned above) bonded through a —S(═O)— moiety or a —S(═O)₂— moiety, respectively, at any position in the alkyl group. Examples include chloromethylsulfinyl, bromomethylsulfoiyl, dichloromethylsulfinyl, trichloromethylsulfinyl, fluoromethylsulfinyl, difluoromethylsulfinyl, trifluoromethylsulfinyl, chlorofluoromethylsulfinyl, dichlorofluoromethylsulfinyl, chlorodifluoromethylsulfinyl, chloromethylsulfonyl, bromomethylsulfonyl, dichloromethylsulfonyl, trichloromethylsulfonyl, fluoromethylsulfonyl, difluoromethylsulfonyl, trifluoromethylsulfonyl, chlorofluoromethylsulfonyl, dichlorofluoromethylsulfonyl, chlorodifluoromethylsulfonyl and the like.

The term “C₂-C₆-alkenyl” as used herein and in the alkenyl moieties of C₂-C₆-alkenyloxy and C₂-C₆-alkenylthio, refers to a branched or unbranched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like.

Accordingly, “C₂-C₆-alkenyloxy” and “C₂-C₆-alkenylthio” as used herein refer to straight-chain or branched alkenyl groups having 2 to 6 carbon atoms (as mentioned above) bonded through an oxygen atom or a sulfur atom respectively, at any position in the alkyl group. Examples include ethenyloxy, ethenylthio and the like.

The term “C₂-C₆-alkynyl” as used herein and in the alkynyl moieties of C₂-C₆-alkynyloxy and C₂-C₆-alkynylthio, refers to a branched or unbranched unsaturated hydrocarbon group having 2 to 6 carbon atoms and containing at least one triple bond, such as ethynyl, propynyl, 1-butynyl, 2-butynyl, and the like.

Accordingly, “C₂-C₆-alkynyloxy” and “C₂-C₆-alkynylthio” as used herein refer to straight-chain or branched alkynyl groups having 2 to 6 carbon atoms (as mentioned above) bonded through an oxygen atom or a sulfur atom respectively, at any position in the alkyl group. Examples include ethynyloxy, ethynylthio and the like.

The term “C₃-C₇-cycloalkyl” as used herein and in the cycloalkyl moiety of C₃-C₇-cycloalkyl-C₁-C₄-alkyl refers to monocyclic 3- to 7-membered saturated carbon atom rings, such as cyclopropyl, cyclobutyl, cyclopentyl, cyclohexyl, or cycloheptyl.

The term “C₃-C₇-cycloalkyl-C₁-C₄-alkyl” as used herein refers to alkyl having 1 to 4 carbon atoms (as mentioned above), wherein one hydrogen atom of the alkyl radical is replaced by a C₃-C₇-cycloalkyl group.

The term “5- or 6-membered heterocycle” as used herein refers to saturated or partially unsaturated heterocyclic radicals having 5 or 6 ring members and 1, 2, 3 or 4, preferably 1, 2 or 3 heteroatoms as ring members (“saturated or partially unsaturated heterocycle”) as well as to monocyclic heteroaromatic radicals which has 5 or 6 ring members, which may be fused to a carbocyclic or heterocyclic 5, 6 or 7 membered ring thus having a total number of ring members from 8 to 10, wherein in each case 1, 2, 3 or 4, preferably 1, 2 or 3, of these ring members are heteroatoms selected, independently from each other, from the group consisting of O, N and S (“hetaryl”). The heterocyclic radical may be attached to the remainder of the molecule via a carbon ring member or via a nitrogen ring member.

Examples for saturated or partially unsaturated heterocycles include pyrrolidinyl, pyrazolinyl, imidazolinyl, pyrrolinyl, pyrazolinyl, imidazolinyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperazinyl, pyranyl, dihydropyranyl, tetrahydropyranyl, 1,3- and 1,4-dioxanyl, thiopyranyl, dihydrothiopyranyl, tetrahydrothiopyranyl, morpholinyl, thiazinyl and the like.

Examples for monocyclic 5- to 6-membered heteroaromatic radicals include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

The term “fused 5 or 6-membered carbocycle or heterocycle” as used herein refers to a partially unsaturated or aromatic carbocyclic or heterocyclic group having 5 or 6 ring members, as defined above, sharing at least one bond between ring members with the cyclic radical to which they are fused, e.g. benzene fused to pyridine is quinoline, benzene fused to pyrimidine is quinazoline, pyridine fused to pyridine is naphtyridine, furane fused to pyridine is furopyridine, dihydrofurane fused to pyridine is dihydrofuropyridine, thiophen fused to pyridine is thienopyridine and dihydrothiophen fused to pyridine is dihydrothienopyridine.

The term “plant propagation material” as used herein includes all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “plants” comprises any types of plants including “non-cultivated plants” and in particular “cultivated plants”.

The term “non-cultivated plants” refers to any wild type species or related species or related genera of a cultivated plant.

The term “cultivated plants” as used herein includes plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transitional modification of protein(s) (oligo- or polypeptides) poly for example by glycosylation or polymer additions such as prenylated, acetylated or farnesylated moieties or PEG moieties (e.g. as disclosed in Biotechnol Prog. 2001 July-August; 17(4):720-8, Protein Eng Des Sel. 2004 January; 17(1):57-66, Nat. Protoc. 2007; 2(5):1225-35, Curr. Opin. Chem. Biol. 2006 October; 10(5):487-91. Epub 2006 Aug. 28, Biomaterials. 2001 March; 22(5):405-17, Bioconjug Chem. 2005 January-February; 16(1):113-21).

The term “cultivated plants” as used herein further includes plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e. g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A-0242236, EP-A-242246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e.g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” as used herein further includes plants that are by the use of recombinant DNA techniques capable to synthesize one or more insecticidal proteins, especially those known from the bacterial genus bacillus, particularly from bacillus thuringiensis, such as ä-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods insects, particularly to beetles (Coleoptera), flies (Diptera), and butterflies and moths (Lepidoptera) and to plant parasitic nematodes (Nematoda).

The term “cultivated plants” as used herein further includes plants that are by the use of recombinant DNA techniques capable to synthesize one or more proteins to increase the resistance or tolerance of those plants to bacterial; viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lyso-zym (e.g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” as used herein further includes plants that are by the use of recombinant DNA techniques capable to synthesize one or more proteins to increase the productivity (e.g. bio mass production, grain yield, starch content, oil content or protein content), tolerance to drought, salinity or other growth-limiting environ-mental factors or tolerance to pests and fungal, bacterial or viral pathogens of those plants.

The term “cultivated plants” as used herein further includes plants that contain by the use of recombinant DNA techniques a modified amount of substances of content or new substances of content, specifically to improve human or animal nutrition, for ex-ample oil crops that produce health-promoting long-chain omega-3 fatty acids or unsaturated omega-9 fatty acids (e.g. Nexera® rape).

The term “cultivated plants” as used herein further includes plants that contain by the use of recombinant DNA techniques a modified amount of substances of content or new substances of content, specifically to improve raw material production, for example potatoes that produce increased amounts of amylopectin (e.g. Amflora® potato).

The remarks made below concerning preferred embodiments of the variables of the compounds of formula (I), of the features of the use and method according to the invention and of the composition of the invention are valid on their own as well as preferably in combination with each other.

Preference is given to sulfonamide compounds of formula (I), wherein R¹ is selected from hydrogen, halogen, C₁-C₆-alkyl, C₃-C₇-cycloalkyl, C₁-C₆-alkoxy, C₁-C₆-alkylthio, C₁-C₆-haloalkyl and C₁-C₆-haloalkoxy. More preferably R¹ is selected from hydrogen, C₁-C₄-alkyl, C₁-C₄-alkoxy, C₁-C₄-haloalkyl and C₁-C₄-haloalkoxy. In particular R¹ is hydrogen, methoxy or methyl, especially hydrogen or methyl.

Preference is further given to sulfonamide compounds of formula (I), wherein R² and R³ are independently selected from hydrogen, halogen, C₁-C₆-alkyl, C₃-C₇-cycloalkyl, C₁-C₆-alkoxy, C₁-C₆-alkylthio, C₁-C₆-haloalkyl and C₁-C₆-haloalkoxy. More preferably R² and R³ are independently selected from hydrogen, C₁-C₄-alkyl, C₁-C₄-alkoxy, C₁-C₄-haloalkyl and C₁-C₄-haloalkoxy. In particular R² and R³ are hydrogen or methyl, especially hydrogen.

Preference is likewise given to sulfonamide compounds of formula (I), wherein R² together with R³ and together with the carbon atoms to which they are bound form a fused 5 or 6-membered carbocycle or a fused 5- or 6-membered heterocycle, which contains 1 heteroatom selected from O, N and S as a ring member, wherein each fused carbocycle or heterocycle is unsubstituted or carries 1 or 2 substituents, independently of one another selected from halogen, cyano, C₁-C₆-alkyl, C₃-C₇-cycloalkyl, C₁-C₆-alkoxy, C₁-C₆-alkylthio, C₁-C₆-haloalkyl and C₁-C₆-haloalkoxy. More preferably in this case R² together with R³ and together with the carbon atoms to which they are bound form a fused dihydrofuran, furan, dihydrothiophene, thiophene, benzene or pyridine, wherein each fused ring is unsubstituted or carries 1 or 2 substituents, independently of one another selected from C₁-C₄-alkyl, C₁-C₄-alkoxy, C₁-C₄-haloalkyl and C₁-C₄-haloalkoxy. In particular R² together with R³ and together with the carbon atoms to which they are bound form a fused benzene, which is unsubstituted or which carries 1 or 2 substituents, independently of one another selected from C₁-C₄-alkyl, C₁-C₄-alkoxy, C₁-C₄-haloalkyl and C₁-C₄-haloalkoxy, especially from methyl or methoxy.

Preference is further given to sulfonamide compounds of formula (I), wherein Y is C(R^{y}) and R^{y} is selected from hydrogen, halogen, C₁-C₆-alkyl, C₃-C₇-cycloalkyl, C₁-C₆-alkoxy, C₁-C₆-alkylthio, C₁-C₆-haloalkyl and C₁-C₆-haloalkoxy. R^{y} in this embodiment is preferably selected from hydrogen, C₁-C₄-alkyl, C₁-C₄-alkoxy, C₁-C₄-haloalkyl and C₁-C₄-haloalkoxy. In particular R^{y} is hydrogen.

Preference is also given to sulfonamide compounds of formula (I), wherein Y is N.

Preference is further given to sulfonamide compounds of formula (I), wherein X is O or NH. In particular X is O.

If n is different from 0, preference is further given to sulfonamide compounds of formula (I), wherein R⁴ is selected from halogen, C₁-C₄-alkyl and C₁-C₄-haloalkyl.

Preference is likewise given to sulfonamide compounds of formula (I), wherein n is 0 or 1. More preferably n is 0.

Preference is further given to sulfonamide compounds of formula (I), wherein Z is a chemical bond or O. In particular Z is a chemical bond.

One preferred embodiment of the present invention relates to sulfonamide compounds of formula (I), wherein R⁵ is selected from phenyl which is unsubstituted or carries 1 or 2 substituents as defined above and which are preferably independently of one another selected from halogen, C₁-C₄-alkyl, C₃-C₇-cycloalkyl, C₁-C₄-alkoxy, C₁-C₄-alkylthio, C₁-C₄-alkylsulfinyl, C₁-C₄-alkylsulfonyl, C₁-C₄-haloalkyl, C₁-C₄-haloalkoxy, C₁-C₄-haloalkylthio, C₁-C₄-haloalkylsulfinyl and C₁-C₄-haloalkylsulfonyl. More preferably R⁵ is phenyl which is unsubstituted or carries 1 or 2 substituents, independently of one another selected from halogen, C₁-C₄-alkyl, C₁-C₄-alkoxy, C₁-C₄-alkylthio, C₁-C₄-alkylsulfonyl, C₁-C₄-haloalkyl, C₁-C₄-haloalkoxy and C₁-C₄-haloalkylthio. In particular R⁵ is phenyl which is unsubstituted or carries 1 or 2 substituents, independently of one another selected from F, Cl, CN, CH₃, C₂H₅, CH(CH₃)₂, CF₃, OCH₃, OC₂H₅, OCHF₂, SCH₃, SCF₃ and SO₂CH₃.

If R⁵ is phenyl which carries 1 substituent, this substituent is preferably attached in the 2-position or in the 4-position relative to the bonding position. If R⁵ is phenyl which carries 2 substituents, these substituents are preferably attached in the 2- and 4-position or alternatively in the 2- and 5-position relative to the bonding position.

Particular preference is given to sulfonamide compounds of formula (I) wherein R⁵ is phenyl which is unsubstituted or carries 1 or 2 substituents, as defined above, and wherein Z is a chemical bond.

Another preferred embodiment of the present invention relates to sulfonamide compounds of formula (I), wherein R⁵ is selected from 5- or 6-membered heterocycles Het, which contain 1, 2 or 3 heteroatoms selected from O, N and S as ring members, wherein Het is unsubstituted or carries 1 or 2 substituents as defined above and wherein the substituents are preferably independently of one another selected from halogen, C₁-C₄-alkyl, C₃-C₇-cycloalkyl, C₁-C₄-alkoxy, C₁-C₄-alkylthio, C₁-C₄-alkylsulfinyl, C₁-C₄-alkylsulfonyl, C₁-C₄-haloalkyl, C₁-C₄-halo-alkoxy, C₁-C₄-haloalkylthio, C₁-C₄-haloalkylsulfinyl and C₁-C₄-haloalkylsulfonyl.

Preferably Het is selected from furanyl (=furyl), pyrrolyl, thiophenyl (=thienyl), oxazolyl, isoxazolyl, pyrazolyl, imidazolyl, thiazolyl, isothiazolyl, 1,2,4-oxadiazolyl, 1,3,4-oxadiazolyl, 1,2,4-thiadiazolyl, 1,3,4-thiadiazolyl, 1,2,3-triazolyl, 1,2,4-triazolyl, pyridinyl, pyrazinyl, pyrimidinyl and pyridazinyl, wherein each of the aforementioned radicals are unsubstituted or carry 1 or 2 substituents as defined above, and wherein the substituents are preferably independently of one another selected from halogen, C₁-C₄-alkyl, C₃-C₇-cycloalkyl, C₁-C₄-alkoxy, C₁-C₄-alkylthio, C₁-C₄-alkylsulfonyl, C₁-C₄-haloalkyl, C₁-C₄-haloalkoxy, C₁-C₄-haloalkylthio, C₁-C₄-haloalkylsulfinyl and C₁-C₄-haloalkylsulfonyl. More preferably Het is selected from isoxazol-3-yl, isothiazol-3-yl, 1,2,4-oxadiazol-3-yl, 1,2,4-thiadiazol-3-yl, pyridin-2-yl and pyridin-3-yl, wherein each of the aforementioned radicals are unsubstituted or carry 1 or 2 substituents, independently of one another selected from halogen, C₁-C₄-alkyl, C₃-C₇-cycloalkyl, C₁-C₄-alkoxy, C₁-C₄-alkylthio, C₁-C₄-alkylsulfonyl, C₁-C₄-haloalkyl, C₁-C₄-haloalkoxy, C₁-C₄-haloalkylthio, C₁-C₄-haloalkylsulfinyl and C₁-C₄-haloalkylsulfonyl. Most preferably Het is selected from pyridin-2-yl which is unsubstituted or carries 1 or 2 of the aforementioned substituents in the 3- and/or 5-position of the pyridine ring.

In particular Het is unsubstituted or carries 1 or 2 substituents independently of one another selected from F, Cl, CN, CH₃, C₂H₅, CH(CH₃)₂, CF₃, OCH₃, OC2H5, OCHF2, SCH₃, SCF₃ and SO₂CH₃.

Particular preference is further given to sulfonamide compounds of formula (I) wherein R⁵ is Het, as defined above, and wherein Z is a O.

Accordingly, one particularly preferred embodiment of the present invention relates to sulfonamide compounds of formula (I), wherein R⁵ is selected from pyridin-2-yl and Z is O, wherein pyridine is unsubstituted or carries 1 or 2 substituents as defined above, which are preferably located in the 3- and/or 5-position of the pyridine ring, and wherein the substituents are as defined above and preferably independently of one another selected from halogen, C₁-C₄-alkyl, C₃-C₇-cycloalkyl, C₁-C₄-alkoxy, C₁-C₄-alkylthio, C₁-C₄-alkylsulfinyl, C₁-C₄-alkylsulfonyl, C₁-C₄-haloalkyl, C₁-C₄-haloalkoxy, C₁-C₄-haloalkylthio, C₁-C₄-haloalkylsulfinyl and C₁-C₄-haloalkylsulfonyl and especially independently of one another selected from F, Cl, CN, CH₃, C₂H₅, CH(CH₃)₂, CF₃, OCH₃, OC₂H₅, OCHF₂, SCH₃, SCF₃ and SO₂CH₃.

Examples of suitable pyridine or pyrimidine moieties (Py)

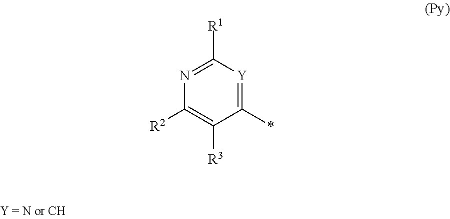

are given in the following table A. In formula (Py) * denotes the bonding site to the remainder of the sulfonamide compound (I).

TABLE A

| (Py) | R¹ | R² | R³ |
|---|---|---|---|
| 1 | H | H | H |
| 2 | Cl | H | H |
| 3 | CH₃ | H | H |
| 4 | C₂H₅ | H | H |
| 5 | CH(CH₃)₂ | H | H |
| 6 | Cyclo- | H | H |
| | propyl | | |
| 7 | CF₃ | H | H |
| 8 | OCH₃ | H | H |
| 9 | OCHF₂ | H | H |
| 10 | SCH₃ | H | H |
| 11 | H | Cl | H |
| 12 | Cl | Cl | H |
| 13 | CH₃ | Cl | H |
| 14 | C₂H₅ | Cl | H |
| 15 | CH(CH₃)₂ | Cl | H |
| 16 | cyclo- | Cl | H |
| | propyl | | |
| 17 | CF₃ | Cl | H |
| 18 | OCH₃ | Cl | H |
| 19 | OCHF₂ | Cl | H |
| 20 | SCH₃ | Cl | H |
| 21 | H | CH₃ | H |
| 22 | Cl | CH₃ | H |
| 23 | CH₃ | CH₃ | H |
| 24 | C₂H₅ | CH₃ | H |
| 25 | CH(CH₃)₂ | CH₃ | H |
| 26 | cyclo- | CH₃ | H |
| | propyl | | |
| 27 | CF₃ | CH₃ | H |
| 28 | OCH₃ | CH₃ | H |
| 29 | OCHF₂ | CH₃ | H |
| 30 | SCH₃ | CH₃ | H |
| 31 | H | C₂H₅ | H |
| 32 | Cl | C₂H₅ | H |
| 33 | CH₃ | C₂H₅ | H |
| 34 | C₂H₅ | C₂H₅ | H |
| 35 | CH(CH₃)₂ | C₂H₅ | H |
| 36 | cyclo- | C₂H₅ | H |
| | propyl | | |
| 37 | CF₃ | C₂H₅ | H |
| 38 | OCH₃ | C₂H₅ | H |
| 39 | OCHF₂ | C₂H₅ | H |
| 40 | SCH₃ | C₂H₅ | H |
| 41 | H | CH(CH₃)₂ | H |
| 42 | Cl | CH(CH₃)₂ | H |
| 43 | CH₃ | CH(CH₃)₂ | H |
| 44 | C₂H₅ | CH(CH₃)₂ | H |
| 45 | CH(CH₃)₂ | CH(CH₃)₂ | H |
| 46 | cyclo- | CH(CH₃)₂ | H |
| | propyl | | |
| 47 | CF₃ | CH(CH₃)₂ | H |
| 48 | OCH₃ | CH(CH₃)₂ | H |
| 49 | OCHF₂ | CH(CH₃)₂ | H |
| 50 | SCH₃ | CH(CH₃)₂ | H |
| 51 | H | cyclo- | H |
| | | propyl | |
| 52 | Cl | cyclo- | H |
| | | propyl | |
| 53 | CH₃ | cyclo- | H |
| | | propyl | |
| 54 | C₂H₅ | cyclo- | H |
| | | propyl | |
| 55 | CH(CH₃)₂ | cyclo- | H |
| | | propyl | |
| 56 | cyclo- | cyclo- | H |
| | propyl | propyl | |
| 57 | CF₃ | cyclo- | H |
| | | propyl | |
| 58 | OCH₃ | cyclo- | H |
| | | propyl | |
| 59 | OCHF₂ | cyclo- | H |
| | | propyl | |
| 60 | SCH₃ | cyclo- | H |
| | | propyl | |
| 61 | H | CF₃ | H |
| 62 | Cl | CF₃ | H |
| 63 | CH₃ | CF₃ | H |
| 64 | C₂H₅ | CF₃ | H |
| 65 | CH(CH₃)₂ | CF₃ | H |
| 66 | cyclo- | CF₃ | H |
| | propyl | | |
| 67 | CF₃ | CF₃ | H |
| 68 | OCH₃ | CF₃ | H |
| 69 | OCHF₂ | CF₃ | H |
| 70 | SCH₃ | CF₃ | H |
| 71 | H | OCH₃ | H |
| 72 | Cl | OCH₃ | H |
| 73 | CH₃ | OCH₃ | H |
| 74 | C₂H₅ | OCH₃ | H |
| 75 | CH(CH₃)₂ | OCH₃ | H |
| 76 | cyclo- | OCH₃ | H |
| | propyl | | |
| 77 | CF₃ | OCH₃ | H |
| 78 | OCH₃ | OCH₃ | H |
| 79 | OCHF₂ | OCH₃ | H |
| 80 | SCH₃ | OCH₃ | H |
| 81 | H | OCHF₂ | H |
| 82 | Cl | OCHF₂ | H |
| 83 | CH₃ | OCHF₂ | H |
| 84 | C₂H₅ | OCHF₂ | H |
| 85 | CH(CH₃)₂ | OCHF₂ | H |
| 86 | cyclo- | OCHF₂ | H |
| | propyl | | |
| 87 | CF₃ | OCHF₂ | H |
| 88 | OCH₃ | OCHF₂ | H |
| 89 | OCHF₂ | OCHF₂ | H |
| 90 | SCH₃ | OCHF₂ | H |
| 91 | H | SCH₃ | H |
| 92 | Cl | SCH₃ | H |
| 93 | CH₃ | SCH₃ | H |
| 94 | C₂H₅ | SCH₃ | H |
| 95 | CH(CH₃)₂ | SCH₃ | H |
| 96 | cyclo- | SCH₃ | H |
| | propyl | | |
| 97 | CF₃ | SCH₃ | H |
| 98 | OCH₃ | SCH₃ | H |
| 99 | OCHF₂ | SCH₃ | H |
| 100 | SCH₃ | SCH₃ | H |
| 101 | H | H | Cl |
| 102 | Cl | H | Cl |
| 103 | CH₃ | H | Cl |
| 104 | C₂H₅ | H | Cl |
| 105 | CH(CH₃)₂ | H | Cl |
| 106 | cyclo- | H | Cl |
| | propyl | | |
| 107 | CF₃ | H | Cl |
| 108 | OCH₃ | H | Cl |
| 109 | OCHF₂ | H | Cl |
| 110 | SCH₃ | H | Cl |
| 111 | H | H | CH₃ |
| 112 | Cl | H | CH₃ |
| 113 | CH₃ | H | CH₃ |
| 114 | C₂H₅ | H | CH₃ |
| 115 | CH(CH₃)₂ | H | CH₃ |
| 116 | cyclo- | H | CH₃ |
| | propyl | | |
| 117 | CF₃ | H | CH₃ |
| 118 | OCH₃ | H | CH₃ |
| 119 | OCHF₂ | H | CH₃ |
| 120 | SCH₃ | H | CH₃ |
| 121 | H | H | C₂H₅ |
| 122 | Cl | H | C₂H₅ |
| 123 | CH₃ | H | C₂H₅ |
| 124 | C₂H₅ | H | C₂H₅ |
| 125 | CH(CH₃)₂ | H | C₂H₅ |
| 126 | cyclo- | H | C₂H₅ |
| | propyl | | |
| 127 | CF₃ | H | C₂H₅ |
| 128 | OCH₃ | H | C₂H₅ |
| 129 | OCHF₂ | H | C₂H₅ |
| 130 | SCH₃ | H | C₂H₅ |
| 131 | H | H | CH(CH₃)₂ |
| 132 | Cl | H | CH(CH₃)₂ |
| 133 | CH₃ | H | CH(CH₃)₂ |
| 134 | C₂H₅ | H | CH(CH₃)₂ |
| 135 | CH(CH₃)₂ | H | CH(CH₃)₂ |
| 136 | cyclo- | H | CH(CH₃)₂ |
| | propyl | | |
| 137 | CF₃ | H | CH(CH₃)₂ |
| 138 | OCH₃ | H | CH(CH₃)₂ |
| 139 | OCHF₂ | H | CH(CH₃)₂ |
| 140 | SCH₃ | H | CH(CH₃)₂ |
| 141 | H | H | cyclo- |
| | | | propyl |
| 142 | Cl | H | cyclo- |
| | | | propyl |
| 143 | CH₃ | H | cyclo- |
| | | | propyl |
| 144 | C₂H₅ | H | cyclo- |
| | | | propyl |
| 145 | CH(CH₃)₂ | H | cyclo- |
| | | | propyl |
| 146 | cyclo- | H | cyclo- |
| | propyl | | propyl |
| 147 | CF₃ | H | cyclo- |
| | | | propyl |
| 148 | OCH₃ | H | cyclo- |
| | | | propyl |
| 149 | OCHF₂ | H | cyclo- |
| | | | propyl |
| 150 | SCH₃ | H | cyclo- |
| | | | propyl |
| 151 | H | H | CF₃ |
| 152 | Cl | H | CF₃ |
| 153 | CH₃ | H | CF₃ |
| 154 | C₂H₅ | H | CF₃ |
| 155 | CH(CH₃)₂ | H | CF₃ |
| 156 | cyclo- | H | CF₃ |
| | propyl | | |
| 157 | CF₃ | H | CF₃ |
| 158 | OCH₃ | H | CF₃ |
| 159 | OCHF₂ | H | CF₃ |
| 160 | SCH₃ | H | CF₃ |
| 161 | H | H | OCH₃ |
| 162 | Cl | H | OCH₃ |
| 163 | CH₃ | H | OCH₃ |
| 164 | C₂H₅ | H | OCH₃ |
| 165 | CH(CH₃)₂ | H | OCH₃ |
| 166 | cyclo- | H | OCH₃ |
| | propyl | | |
| 167 | CF₃ | H | OCH₃ |
| 168 | OCH₃ | H | OCH₃ |
| 169 | OCHF₂ | H | OCH₃ |
| 170 | SCH₃ | H | OCH₃ |
| 171 | H | H | OCHF₂ |
| 172 | Cl | H | OCHF₂ |
| 173 | CH₃ | H | OCHF₂ |
| 174 | C₂H₅ | H | OCHF₂ |
| 175 | CH(CH₃)₂ | H | OCHF₂ |
| 176 | cyclo- | H | OCHF₂ |
| | propyl | | |
| 177 | CF₃ | H | OCHF₂ |
| 178 | OCH₃ | H | OCHF₂ |
| 179 | OCHF₂ | H | OCHF₂ |
| 180 | SCH₃ | H | OCHF₂ |
| 181 | H | H | SCH₃ |
| 182 | Cl | H | SCH₃ |
| 183 | CH₃ | H | SCH₃ |
| 184 | C₂H₅ | H | SCH₃ |
| 185 | CH(CH₃)₂ | H | SCH₃ |
| 186 | cyclo- | H | SCH₃ |
| | propyl | | |
| 187 | CF₃ | H | SCH₃ |
| 188 | OCH₃ | H | SCH₃ |
| 189 | OCHF₂ | H | SCH₃ |
| 190 | SCH₃ | H | SCH₃ |
| 191 | H | Cl | Cl |
| 192 | H | CH₃ | Cl |
| 193 | H | C₂H₅ | Cl |
| 194 | H | CH(CH₃)₂ | Cl |
| 195 | H | cyclo- | Cl |
| | | propyl | |
| 196 | H | CF₃ | Cl |
| 197 | H | OCH₃ | Cl |
| 198 | H | OCHF₂ | Cl |
| 199 | H | SCH₃ | Cl |
| 200 | H | Cl | CH₃ |
| 201 | H | CH₃ | CH₃ |
| 202 | H | C₂H₅ | CH₃ |
| 203 | H | CH(CH₃)₂ | CH₃ |
| 204 | H | cyclo- | CH₃ |
| | | propyl | |
| 205 | H | CF₃ | CH₃ |
| 206 | H | OCH₃ | CH₃ |
| 207 | H | OCHF₂ | CH₃ |
| 208 | H | SCH₃ | CH₃ |
| 209 | H | Cl | C₂H₅ |
| 210 | H | CH₃ | C₂H₅ |
| 211 | H | C₂H₅ | C₂H₅ |
| 212 | H | CH(CH₃)₂ | C₂H₅ |
| 213 | H | cyclo- | C₂H₅ |
| | | propyl | |
| 214 | H | CF₃ | C₂H₅ |
| 215 | H | OCH₃ | C₂H₅ |
| 216 | H | OCHF₂ | C₂H₅ |
| 217 | H | SCH₃ | C₂H₅ |
| 218 | H | Cl | CH(CH₃)₂ |
| 219 | H | CH₃ | CH(CH₃)₂ |
| 220 | H | C₂H₅ | CH(CH₃)₂ |
| 221 | H | CH(CH₃)₂ | CH(CH₃)₂ |
| 222 | H | cyclo- | CH(CH₃)₂ |
| | | propyl | |
| 223 | H | CF₃ | CH(CH₃)₂ |
| 224 | H | OCH₃ | CH(CH₃)₂ |
| 225 | H | OCHF₂ | CH(CH₃)₂ |
| 226 | H | SCH₃ | CH(CH₃)₂ |
| 227 | H | Cl | cyclo- |
| | | | propyl |
| 228 | H | CH₃ | cyclo- |
| | | | propyl |
| 229 | H | C₂H₅ | cyclo- |
| | | | propyl |
| 230 | H | CH(CH₃)₂ | cyclo- |
| | | | propyl |
| 231 | H | cyclo- | cyclo- |
| | | propyl | propyl |
| 232 | H | CF₃ | cyclo- |
| | | | propyl |
| 233 | H | OCH₃ | cyclo- |
| | | | propyl |
| 234 | H | OCHF₂ | cyclo- |
| | | | propyl |
| 235 | H | SCH₃ | cyclo- |
| | | | propyl |
| 236 | H | Cl | CF₃ |
| 237 | H | CH₃ | CF₃ |
| 238 | H | C₂H₅ | CF₃ |
| 239 | H | CH(CH₃)₂ | CF₃ |
| 240 | H | cyclo- | CF₃ |
| | | propyl | |
| 241 | H | CF₃ | CF₃ |
| 242 | H | OCH₃ | CF₃ |
| 243 | H | OCHF₂ | CF₃ |
| 244 | H | SCH₃ | CF₃ |
| 245 | H | Cl | OCH₃ |
| 246 | H | CH₃ | OCH₃ |
| 247 | H | C₂H₅ | OCH₃ |
| 248 | H | CH(CH₃)₂ | OCH₃ |
| 249 | H | cyclo- | OCH₃ |
| | | propyl | |
| 250 | H | CF₃ | OCH₃ |
| 251 | H | CF₃ | OCH₃ |
| 252 | H | OCH₃ | OCH₃ |
| 253 | H | OCHF₂ | OCH₃ |
| 254 | H | SCH₃ | OCH₃ |
| 255 | H | Cl | OCHF₂ |
| 256 | H | CH₃ | OCHF₂ |
| 257 | H | C₂H₅ | OCHF₂ |
| 258 | H | CH(CH₃)₂ | OCHF₂ |
| 259 | H | cyclo- | OCHF₂ |
| | | propyl | |
| 260 | H | CF₃ | OCHF₂ |
| 261 | H | OCH₃ | OCHF₂ |
| 262 | H | OCHF₂ | OCHF₂ |
| 263 | H | SCH₃ | OCHF₂ |
| 264 | H | Cl | SCH₃ |
| 265 | H | CH₃ | SCH₃ |
| 266 | H | C₂H₅ | SCH₃ |
| 267 | H | CH(CH₃)₂ | SCH₃ |
| 268 | H | cyclo- | SCH₃ |
| | | propyl | |
| 269 | H | CF₃ | SCH₃ |
| 270 | H | OCH₃ | SCH₃ |
| 271 | H | OCHF₂ | SCH₃ |
| 272 | H | SCH₃ | SCH₃ |

| | | |
|---|---|---|
| 273 | H | —CH═CH—CH═CH— |
| 274 | H | —CH═CH—CH═N— |
| 275 | H | —O—CH₂—CH₂— |
| 276 | H | —O—CH═CH— |
| 277 | H | —S—CH₂—CH₂— |
| 278 | H | —S—CH═CH— |
| 279 | H | —CH₂—CH₂—O— |
| 280 | H | —CH═CH—O— |
| 281 | H | —CH₂—CH₂—S— |
| 282 | H | —CH═CH—S— |

Examples of suitable radicals R⁵ are the radicals of formula (Ar),

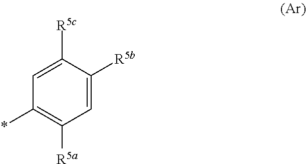

wherein R^{5a}, R^{5b} and R^{5c} have the meaning given in the following table B. In formula (Ar) * denotes the bonding site to the remainder of the sulfonamide compound (I).

TABLE B

| Ar | R^{5a} | R^{5b} | R^{5c} |
|---|---|---|---|
| 1 | H | H | H |
| 2 | F | H | H |
| 3 | Cl | H | H |
| 4 | CH₃ | H | H |
| 5 | CF₃ | H | H |
| 6 | H | F | H |
| 7 | H | Cl | H |
| 8 | H | CN | H |
| 9 | H | CH₃ | H |
| 10 | H | C₂H₅ | H |
| 11 | H | CH(CH₃)₂ | H |
| 12 | H | CF₃ | H |
| 13 | H | OCH₃ | H |
| 14 | H | OC₂H₅ | H |
| 15 | H | OCHF₂ | H |
| 16 | H | SCH₃ | H |
| 17 | H | SCF₃ | H |
| 18 | H | SO₂CH₃ | H |
| 19 | F | F | H |
| 20 | F | Cl | H |
| 21 | F | CN | H |
| 22 | F | CH₃ | H |
| 23 | F | C₂H₅ | H |
| 24 | F | CH(CH₃)₂ | H |
| 25 | F | CF₃ | H |
| 26 | F | OCH₃ | H |
| 27 | F | OC₂H₅ | H |
| 28 | F | OCHF₂ | H |
| 29 | F | SCH₃ | H |
| 30 | F | SCF₃ | H |
| 31 | F | SO₂CH₃ | H |
| 32 | Cl | F | H |
| 33 | Cl | Cl | H |
| 34 | Cl | CN | H |
| 35 | Cl | CH₃ | H |
| 36 | Cl | C₂H₅ | H |
| 37 | Cl | CH(CH₃)₂ | H |
| 38 | Cl | CF₃ | H |
| 39 | Cl | OCH₃ | H |
| 40 | Cl | OC₂H₅ | H |
| 41 | Cl | OCHF₂ | H |
| 42 | Cl | SCH₃ | H |
| 43 | Cl | SCF₃ | H |
| 44 | Cl | SO₂CH₃ | H |
| 45 | CH₃ | F | H |
| 46 | CH₃ | Cl | H |
| 47 | CH₃ | CN | H |
| 48 | CH₃ | CH₃ | H |
| 49 | CH₃ | C₂H₅ | H |
| 50 | CH₃ | CH(CH₃)₂ | H |
| 51 | CH₃ | CF₃ | H |
| 52 | CH₃ | OCH₃ | H |
| 53 | CH₃ | OC₂H₅ | H |
| 54 | CH₃ | OCHF₂ | H |
| 55 | CH₃ | SCH₃ | H |
| 56 | CH₃ | SCF₃ | H |
| 57 | CH₃ | SO₂CH₃ | H |
| 58 | CF₃ | F | H |
| 59 | CF₃ | Cl | H |
| 60 | CF₃ | CN | H |
| 61 | CF₃ | CH₃ | H |
| 62 | CF₃ | C₂H₅ | H |
| 63 | CF₃ | CH(CH₃)₂ | H |
| 64 | CF₃ | CF₃ | H |
| 65 | CF₃ | OCH₃ | H |
| 66 | CF₃ | OC₂H₅ | H |
| 67 | CF₃ | OCHF₂ | H |
| 68 | CF₃ | SCH₃ | H |
| 69 | CF₃ | SCF₃ | H |
| 70 | CF₃ | SO₂CH₃ | H |
| 71 | F | H | F |
| 72 | F | H | Cl |
| 73 | F | H | CN |
| 74 | F | H | CH₃ |
| 75 | F | H | C₂H₅ |
| 76 | F | H | CH(CH₃)₂ |
| 77 | F | H | CF₃ |
| 78 | F | H | OCH₃ |
| 79 | F | H | OC₂H₅ |
| 80 | F | H | OCHF₂ |
| 81 | F | H | SCH₃ |
| 82 | F | H | SCF₃ |
| 83 | F | H | SO₂CH₃ |
| 84 | Cl | H | F |
| 85 | Cl | H | Cl |
| 86 | Cl | H | CN |
| 87 | Cl | H | CH₃ |
| 88 | Cl | H | C₂H₅ |
| 89 | Cl | H | CH(CH₃)₂ |
| 90 | Cl | H | CF₃ |
| 91 | Cl | H | OCH₃ |
| 92 | Cl | H | OC₂H₅ |
| 93 | Cl | H | OCHF₂ |
| 94 | Cl | H | SCH₃ |
| 95 | Cl | H | SCF₃ |
| 96 | Cl | H | SO₂CH₃ |
| 97 | CH₃ | H | F |
| 98 | CH₃ | H | Cl |
| 99 | CH₃ | H | CN |
| 100 | CH₃ | H | CH₃ |
| 101 | CH₃ | H | CH₂H₅ |
| 102 | CH₃ | H | CH(CH₃)₂ |
| 103 | CH₃ | H | CF₃ |
| 104 | CH₃ | H | OCH₃ |
| 105 | CH₃ | H | OC₂H₅ |
| 106 | CH₃ | H | OCHF₂ |
| 107 | CH₃ | H | SCH₃ |
| 108 | CH₃ | H | SCF₃ |
| 109 | CH₃ | H | SO₂CH₃ |
| 110 | CF₃ | H | F |
| 111 | CF₃ | H | Cl |
| 112 | CF₃ | H | CN |
| 113 | CF₃ | H | CH₃ |
| 114 | CF₃ | H | C₂H₅ |
| 115 | CF₃ | H | CH(CH₃)₂ |
| 116 | CF₃ | H | CF₃ |
| 117 | CF₃ | H | OCH₃ |
| 118 | CF₃ | H | OC₂H₅ |
| 119 | CF₃ | H | OCHF₂ |
| 120 | CF₃ | H | SCH₃ |
| 121 | CF₃ | H | SCF₃ |
| 122 | CF₃ | H | SO₂CH₃ |

Further examples of suitable radicals R⁵ are the radicals Het given in the following table C.

TABLE C

| Het | R⁵ |
|---|---|
| 1 | pyridin-2-yl |
| 2 | 3-fluoropyridin-2-yl |
| 3 | 3-chloropyridin-2-yl |
| 4 | 3-cyanopyridin-2-yl |
| 5 | 3-methylpyridin-2-yl |
| 6 | 3-ethylpyridin-2-yl |
| 7 | 3-isopropylpyridin-2-yl |
| 8 | 3-trifluoromethylpyridin-2-yl |
| 9 | 3-methoxypyridin-2-yl |
| 10 | 3-ethoxypyridin-2-yl |
| 11 | 3-difluoromethoxypyridin-2-yl |
| 12 | 3-methylthiopyridin-2-yl |
| 13 | 3-trifluoromethylthiopyridin-2-yl |
| 14 | 3-methylsulfonylpyridin-2-yl |
| 15 | 5-fluoropyridin-2-yl |
| 16 | 5-chloropyridin-2-yl |
| 17 | 5-cyanopyridin-2-yl |
| 18 | 5-methylpyridin-2-yl |
| 19 | 5-ethylpyridin-2-yl |
| 20 | 5-isopropylpyridin-2-yl |
| 21 | 5-trifluoromethylpyridin-2-yl |
| 22 | 5-methoxypyridin-2-yl |
| 23 | 5-ethoxypyridin-2-yl |
| 24 | 5-difluoromethoxypyridin-2-yl |
| 25 | 5-methylthiopyridin-2-yl |
| 26 | 5-trifluoromethylthiopyridin-2-yl |
| 27 | 5-methylsulfonylpyridin-2-yl |
| 28 | 3,5-difluoropyridin-2-yl |
| 29 | 3-fluoro-5-chloropyridin-2-yl |
| 30 | 3-fluoro-5-cyanopyridin-2-yl |
| 31 | 3-fluoro-5-methylpyridin-2-yl |
| 32 | 3-fluoro-5-ethylpyridin-2-yl |
| 33 | 3-fluoro-5-isopropylpyridin-2-yl |
| 34 | 3-fluoro-5-trifluoromethylpyridin-2-yl |
| 35 | 3-fluoro-5-methoxypyridin-2-yl |
| 36 | 3-fluoro-5-ethoxypyridin-2-yl |
| 37 | 3-fluoro-5-difluoromethoxypyridin-2-yl |
| 38 | 3-fluoro-5-methylthiopyridin-2-yl |
| 39 | 3-fluoro-5-trifluoromethylthiopyridin-2-yl |
| 40 | 3-fluoro-5-methylsulfonylpyridine-2-yl |
| 41 | 3-chloro-5-fluoropyridin-2-yl |
| 42 | 3,5-dichloropyridin-2-yl |
| 43 | 3-chloro-5-cyanopyridin-2-yl |
| 44 | 3-chloro-5-methylpyridin-2-yl |
| 45 | 3-chloro-5-ethylpyridin-2-yl |
| 46 | 3-chloro-5-isopropylpyridin-2-yl |
| 47 | 3-chloro-5-trifluoromethylpyridin-2-yl |
| 48 | 3-chloro-5-methoxypyridin-2-yl |
| 49 | 3-chloro-5-ethoxypyridin-2-yl |
| 50 | 3-chloro-5-difluoromethoxypyridin-2-yl |
| 51 | 3-chloro-5-methylthiopyridin-2-yl |
| 52 | 3-chloro-5-trifluoromethylthiopyridin-2-yl |
| 53 | 3-chloro-5-methylsulfonylpyridin-2-yl |
| 54 | 3-cyano-5-fluoropyridin-2-yl |
| 55 | 3-cyano-5-chloropyridin-2-yl |
| 56 | 3,5-dicyanopyridin-2-yl |
| 57 | 3-cyano-5-methylpyridin-2-yl |
| 58 | 3-cyano-5-ethylpyridin-2-yl |
| 59 | 3-cyano-5-isopropylpyridin-2-yl |
| 60 | 3-cyano-5-trifluoromethylpyridin-2-yl |
| 61 | 3-cyano-5-methoxypyridin-2-yl |
| 62 | 3-cyano-5-ethoxypyridin-2-yl |
| 63 | 3-cyano-5-difluoromethoxypyridin-2-yl |
| 64 | 3-cyano-5-methylthiopyridin-2-yl |
| 65 | 3-cyano-5-trifluoromethylthiopyridin-2-yl |
| 66 | 3-cyano-5-methylsulfonylpyridin-2-yl |
| 67 | 3-methyl-5-fluoropyridin-2-yl |
| 68 | 3-methyl-5-chloropyridin-2-yl |
| 69 | 3-methyl-5-cyanopyridin-2-yl |
| 70 | 3,5-dimethylpyridin-2-yl |
| 71 | 3-methyl-5-ethylpyridin-2-yl |
| 72 | 3-methyl-5-isopropylpyridin-2-yl |
| 73 | 3-methyl-5-trifluoromethylpyridin-2-yl |
| 74 | 3-methyl-5-methoxypyridin-2-yl |
| 75 | 3-methyl-5-ethoxypyridin-2-yl |
| 76 | 3-methyl-5-difluoromethoxypyridin-2-yl |
| 77 | 3-methyl-5-methylthiopyridin-2-yl |
| 78 | 3-methyl-5-trifluoromethylthiopyridin-2-yl |
| 79 | 3-methyl-5-methylsulfonylpyridin-2-yl |
| 80 | 3-ethyl-5-fluoropyridin-2-yl |
| 81 | 3-ethyl-5-chloropyridin-2-yl |
| 82 | 3-ethyl-5-cyanopyridin-2-yl |
| 83 | 3-ethyl-5-methylpyridin-2-yl |
| 84 | 3,5-diethylpyridin-2-yl |
| 85 | 3-ethyl-5-isopropylpyridin-2-yl |
| 86 | 3-ethyl-5-trifluoromethylpyridin-2-yl |
| 87 | 5-methoxypyridin-2-yl |
| 88 | 5-ethoxypyridin-2-yl |
| 89 | 3-ethyl-5-difluoromethoxypyridin-2-yl |
| 90 | 3-ethyl-5-methylthiopyridin-2-yl |
| 91 | 3-ethyl-5-trifluoromethylthiopyridin-2-yl |
| 92 | 3-ethyl-5-methylsulfonylpyridin-2-yl |
| 93 | 3-isopropyl-5-fluoropyridin-2-yl |
| 94 | 3-isopropyl-5-chloropyridin-2-yl |
| 95 | 3-isopropyl-5-cyanopyridin-2-yl |
| 96 | 3-isopropyl-5-methylpyridin-2-yl |
| 97 | 3-isopropyl-5-ethylpyridin-2-yl |
| 98 | 3,5-diisopropylpyridin-2-yl |
| 99 | 3-isopropyl-5-trifluoromethylpyridin-2-yl |
| 100 | 3-isopropyl-5-methoxypyridin-2-yl |
| 101 | 3-isopropyl-5-ethoxypyridin-2-yl |
| 102 | 3-isopropyl-5-difluoromethoxypyridin-2-yl |
| 103 | 3-isopropyl-5-methylthiopyridin-2-yl |
| 104 | 3-isopropyl-5-trifluoromethylthiopyridin-2-yl |
| 105 | 3-isopropyl-5-methylsulfonylpyridin-2-yl |
| 106 | 3-trifluoromethyl-5-fluoropyridin-2-yl |
| 107 | 3-trifluoromethyl-5-chloropyridin-2-yl |
| 108 | 3-trifluoromethyl-5-cyanopyridin-2-yl |
| 109 | 3-trifluoromethyl-5-methylpyridin-2-yl |
| 110 | 3-trifluoromethyl-5-ethylpyridin-2-yl |
| 111 | 3-trifluoromethyl-5-isopropylpyridin-2-yl |
| 112 | 3,5-di(trifluoromethyl)pyridin-2-yl |
| 113 | 3-trifluoromethyl-5-methoxypyridin-2-yl |
| 114 | 3-trifluoromethyl-5-ethoxypyridin-2-yl |
| 115 | 3-trifluoromethyl-5-difluoromethoxypyridin-2-yl |
| 116 | 3-trifluoromethyl-5-methylthiopyridin-2-yl |
| 117 | 3-trifluoromethyl-5-trifluoromethylthio-pyridin-2-yl |
| 118 | 3-trifluoromethyl-5-methylsulfonyl-pyridin-2-yl |
| 119 | 3-methoxy-5-fluoropyridin-2-yl |
| 120 | 3-methoxy-5-chloropyridin-2-yl |
| 121 | 3-methoxy-5-cyanopyridin-2-yl |
| 122 | 3-methoxy-5-methylpyridin-2-yl |
| 123 | 3-methoxy-5-ethylpyridin-2-yl |
| 124 | 3-methoxy-5-isopropylpyridin-2-yl |
| 125 | 3-methoxy-5-trifluoromethylpyridin-2-yl |
| 126 | 3,5-dimethoxypyridin-2-yl |
| 127 | 3-methoxy-5-ethoxypyridin-2-yl |
| 128 | 3-methoxy-5-difluoromethoxypyridin-2-yl |
| 129 | 3-methoxy-5-methylthiopyridin-2-yl |
| 130 | 3-methoxy-5-trifluoromethylthio-pyridin-2-yl |
| 131 | 3-methoxy-5-methylsulfonylpyridin-2-yl |
| 132 | 3-ethoxy-5-fluoropyridin-2-yl |
| 133 | 3-ethoxy-5-chloropyridin-2-yl |
| 134 | 3-ethoxy-5-cyanopyridin-2-yl |
| 135 | 3-ethoxy-5-methylpyridin-2-yl |
| 136 | 3-ethoxy-5-ethylpyridin-2-yl |
| 137 | 3-ethoxy-5-isopropylpyridin-2-yl |
| 138 | 3-ethoxy-5-trifluoromethylpyridin-2-yl |
| 139 | 3-ethoxy-5-methoxypyridin-2-yl |
| 140 | 3,5-diethoxypyridin-2-yl |
| 141 | 3-ethoxy-5-difluoromethoxypyridin-2-yl |
| 142 | 3-ethoxy-5-methylthiopyridin-2-yl |
| 143 | 3-ethoxy-5-trifluoromethylthiopyridin-2-yl |
| 144 | 3-ethoxy-5-methylsulfonylpyridin-2-yl |
| 145 | 3-difluoromethoxy-5-fluoropyridin-2-yl |
| 146 | 3-difluoromethoxy-5-chloropyridin-2-yl |
| 147 | 3-difluoromethoxy-5-cyanopyridin-2-yl |
| 148 | 3-difluoromethoxy-5-methylpyridin-2-yl |
| 149 | 3-difluoromethoxy-5-ethylpyridin-2-yl |
| 150 | 3-difluoromethoxy-5-isopropylpyridin-2-yl |
| 151 | 3-difluoromethoxy-5-trifluoromethyl-pyridin-2-yl |
| 152 | 3-difluoromethoxy-5-methoxypyridin-2-yl |
| 153 | 3-difluoromethoxy-5-ethoxypyridin-2-yl |
| 154 | 3,5-di(difluoromethoxy)pyridin-2-yl |
| 155 | 3-difluoromethoxy-5-methylthio-pyridin-2-yl |
| 156 | 3-difluoromethoxy-5-trifluoromethylthiopyridin-2-yl |
| 157 | 3-difluoromethoxy-5-methylsulfonyl-pyridin-2-yl |
| 158 | 3-methylthio-5-fluoropyridin-2-yl |
| 159 | 3-methylthio-5-chloropyridin-2-yl |
| 160 | 3-methylthio-5-cyanopyridin-2-yl |
| 161 | 3-methylthio-5-methylpyridin-2-yl |
| 162 | 3-methylthio-5-ethylpyridin-2-yl |
| 163 | 3-methylthio-5-isopropylpyridin-2-yl |
| 164 | 3-methylthio-5-trifluoromethylpyridin-2-yl |
| 165 | 3-methylthio-5-methoxypyridin-2-yl |
| 166 | 3-methylthio-5-ethoxypyridin-2-yl |
| 167 | 3-methylthio-5-difluoromethoxy-pyridin-2-yl |
| 168 | 3,5-di(methylthio)pyridin-2-yl |
| 169 | 3-methylthio-5-trifluoromethylthio-pyridin-2-yl |
| 170 | 3-methylthio-5-methylsulfonylpyridin-2-yl |
| 171 | 3-trifluoromethylthio-5-fluoropyridin-2-yl |
| 172 | 3-trifluoromethylthio-5-chloropyridin-2-yl |
| 173 | 3-trifluoromethylthio-5-cyanopyridin-2-yl |
| 174 | 3-trifluoromethylthio-5-methylpyridin-2-yl |
| 175 | 3-trifluoromethylthio-5-ethylpyridin-2-yl |
| 176 | 3-trifluoromethylthio-5-isopropyl-pyridin-2-yl |
| 177 | 3-trifluoromethylthio-5-trifluoromethyl-pyridin-2-yl |
| 178 | 3-trifluoromethylthio-5-methoxy-pyridin-2-yl |
| 179 | 3-trifluoromethylthio-5-ethoxypyridin-2-yl |
| 180 | 3-trifluoromethylthio-5-difluoro-methoxypyridin-2-yl |
| 181 | 3-trifluoromethylthio-5-methylthio-pyridin-2-yl |
| 182 | 3,5-di(trifluoromethylthio)pyridin-2-yl |
| 183 | 3-trifluoromethylthio-5-methylsulfonyl-pyridin-2-yl |
| 184 | 3-methylsulfonyl-5-fluoropyridin-2-yl |
| 185 | 3-methylsulfonyl-5-chloropyridin-2-yl |
| 186 | 3-methylsulfonyl-5-cyanopyridin-2-yl |
| 187 | 3-methylsulfonyl-5-methylpyridin-2-yl |
| 188 | 3-methylsulfonyl-5-ethylpyridin-2-yl |
| 189 | 3-methylsulfonyl-5-isopropyl-pyridin-2-yl |
| 190 | 3-methylsulfonyl-5-trifluoromethyl-pyridin-2-yl |
| 191 | 3-methylsulfonyl-5-methoxypyridin-2-yl |
| 192 | 3-methylsulfonyl-5-ethoxypyridin-2-yl |
| 193 | 3-methylsulfonyl-5-difluoromethoxy-pyridin-2-yl |
| 194 | 3-methylsulfonyl-5-methylthiopyridin-2-yl |
| 195 | 3-methylsulfonyl-5-trifluoromethylthio-pyridin-2-yl |
| 196 | 3,5-di(methylsulfonyl)pyridin-2-yl |
| 197 | pyridin-3-yl |
| 198 | 6-fluoropyridin-3-yl |
| 199 | 6-chloropyridin-3-yl |
| 200 | 6-cyanopyridin-3-yl |
| 201 | 6-methylpyridin-3-yl |
| 202 | 6-ethylpyridin-3-yl |
| 203 | 6-isopropylpyridin-3-yl |
| 204 | 6-trifluoromethylpyridin-3-yl |
| 205 | 6-methoxypyridin-3-yl |
| 206 | 6-ethoxypyridin-3-yl |
| 207 | 6-difluoromethoxypyridin-3-yl |
| 208 | 6-methylthiopyridin-3-yl |
| 209 | 6-trifluoromethylthiopyridin-3-yl |
| 210 | 6-methylsulfonylpyridin-3-yl |
| 211 | isoxazol-3-yl |
| 212 | 5-fluoroisoxazol-3-yl |
| 213 | 5-chloroisoxazol-3-yl |
| 214 | 5-cyanoisoxazol-3-yl |
| 215 | 5-methylisoxazol-3-yl |
| 216 | 5-ethylisoxazol-3-yl |
| 217 | 5-isopropylisoxazol-3-yl |
| 218 | 5-trifluoromethylisoxazol-3-yl |
| 219 | 5-methoxyisoxazol-3-yl |
| 220 | 5-ethoxyisoxazol-3-yl |
| 221 | 5-difluoromethoxyisoxazol-3-yl |
| 222 | 5-methylthioisoxazol-3-yl |
| 223 | 5-trifluoromethylthioisoxazol-3-yl |
| 224 | 5-methylsulfonylisoxazol-3-yl |
| 225 | isothiazol-3-yl |
| 226 | 5-fluoroisothiazol-3-yl |
| 227 | 5-chloroisothiazol-3-yl |
| 228 | 5-cyanoisothiazol-3-yl |
| 229 | 5-methylisothiazol-3-yl |
| 230 | 5-ethylisothiazol-3-yl |
| 231 | 5-isopropylisothiazol-3-yl |
| 232 | 5-trifluoromethylisothiazol-3-yl |
| 233 | 5-methoxyisothiazol-3-yl |
| 234 | 5-ethoxyisothiazol-3-yl |
| 235 | 5-difluoromethoxyisothiazol-3-yl |
| 236 | 5-methylthioisothiazol-3-yl |
| 237 | 5-trifluoromethylthioisothiazol-3-yl |
| 238 | 5-methylsulfonylisothiazol-3-yl |
| 239 | 1,2,4-oxadiazol-3-yl |
| 240 | 5-fluoro-1,2,4-oxadiazol-3-yl |
| 241 | 5-chloro-1,2,4-oxadiazol-3-yl |
| 242 | 5-cyano-1,2,4-oxadiazol-3-yl |
| 243 | 5-methyl-1,2,4-oxadiazol-3-yl |
| 244 | 5-ethyl-1,2,4-oxadiazol-3-yl |
| 245 | 5-isopropyl-1,2,4-oxadiazol-3-yl |
| 246 | 5-trifluoromethyl-1,2,4-oxadiazol-3-yl |
| 247 | 5-methoxy-1,2,4-oxadiazol-3-yl |
| 248 | 5-ethoxy-1,2,4-oxadiazol-3-yl |
| 249 | 5-difluoromethoxy-1,2,4-oxadiazol-3-yl |
| 250 | 5-methylthio-1,2,4-oxadiazol-3-yl |
| 251 | 5-trifluoromethylthio-1,2,4-oxadiazol-3-yl |
| 252 | 5-methylsulfonyl-1,2,4-oxadiazol-3-yl |
| 253 | 1,2,4-thiadiazol-3-yl |
| 254 | 5-fluoroisoxazol-3-yl |
| 255 | 5-chloroisoxazol-3-yl |
| 256 | 5-cyanoisoxazol-3-yl |
| 257 | 5-methylisoxazol-3-yl |
| 258 | 5-ethylisoxazol-3-yl |
| 259 | 5-isopropylisoxazol-3-yl |
| 260 | 5-trifluoromethylisoxazol-3-yl |
| 261 | 5-methoxyisoxazol-3-yl |
| 262 | 5-ethoxyisoxazol-3-yl |
| 263 | 5-difluoromethoxyisoxazol-3-yl |
| 264 | 5-methylthioisoxazol-3-yl |
| 265 | 5-trifluoromethylthioisoxazol-3-yl |
| 266 | 5-methylsulfonylisoxazol-3-yl |

With respect to their use, particular preference is given to sulfonamide compounds of formula (I) compiled in the tables below. Moreover, the groups mentioned as substituents in the tables are on their own, independently of the combination in which they are mentioned, a particularly preferred embodiment of the substituent in question.

One particular embodiment of the invention relates to sulfonamide compounds of formula (I), wherein m is 0, X is O, Y is CH and Z is a chemical bond, in the following referred to as sulfonamide compounds of formula (I.A),

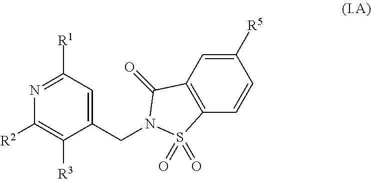

wherein R¹, R², R³ and R⁵ have the meanings given above, especially those meanings mentioned as being preferred.

### Table 1 (Compounds (I.A-Ar.1-1) to (I.A-Ar.1-282))

Compounds of formula (I.A), wherein R⁵ is a radical (Ar.1) as defined in line 1 of table B and R¹, R² and R³ have one of the meanings corresponding to any line of table A.

### Tables 2 to 122 (Compounds (I.A-Ar.2-1) to (I.A-Ar.122-282))

Compounds of formula (I.A), wherein R⁵ is one of the radicals (Ar.2) to (Ar.122) as defined in lines 2 to 122 of table B and R¹, R² and R³ have one of the meanings corresponding to any line of table A.

### Table 123 (Compounds (I.A-Het.1-1) to (I.A-Het.1-282))

Compounds of formula (I.A), wherein R⁵ is a radical (Het.1) as defined in line 1 of table C and R¹, R² and R³ have one of the meanings corresponding to any line of table A.

### Tables 124 to 388 (Compounds (I.A-Het.2-1) to (I.A-Het.266-282))

Compounds of formula (I.A), wherein R⁵ is one of the radicals (Het.2) to (Het.266) as defined in lines 2 to 266 of table C and R¹, R² and R³ have one of the meanings corresponding to any line of table A.

Another particular embodiment of the invention relates to sulfonamide compounds of formula (I), wherein m is 0, X is O, Y is CH and Z is O, in the following referred to as sulfonamide compounds of formula (I.B),

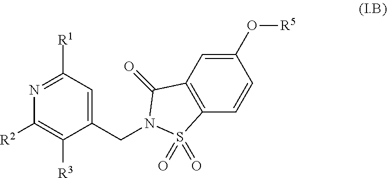

wherein R¹, R², R³ and R⁵ have the meanings given above, especially those meanings mentioned as being preferred.

### Table 389 (Compounds (I.B-Ar.1-1) to (I.B-Ar.1-282))

Compounds of formula (I.B), wherein R⁵ is a radical (Ar.1) as defined in line 1 of table B and R¹, R² and R³ have one of the meanings corresponding to any line of table A.

### Tables 390 to 510 (Compounds (I.B-Ar.2-1) to (I.B-Ar.122-282))

Compounds of formula (I.B), wherein R⁵ is one of the radicals (Ar.2) to (Ar.122) as defined in lines 2 to 122 of table B and R¹, R² and R³, have one of the meanings corresponding to any line of table A.

### Table 511 (Compounds (I.B-Het.1-1) to (I.B-Het.1-282))

Compounds of formula (I.B), wherein R⁵ is a radical (Het.1) as defined in line 1 of table C and R¹, R² and R³ have one of the meanings corresponding to any line of table A.

### Tables 512 to 776 (Compounds (I.B-Het.2-1) to (I.B-Het.266-282))

Compounds of formula (I.B), wherein R⁵ is one of the radicals (Het.2) to (Het.266) as defined in lines 2 to 266 of table C and R¹, R² and R³ have one of the meanings corresponding to any line of table A.

Another particular embodiment of the invention relates to sulfonamide compounds of formula (I), wherein m is 0, X is O, Y is N and Z is a chemical bond, in the following referred to as sulfonamide compounds of formula (I.C),

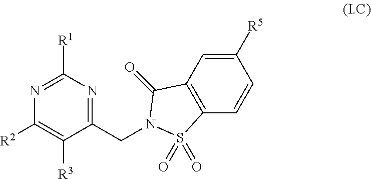

wherein R¹, R², R³ and R⁵ have the meanings given above, especially those meanings mentioned as being preferred.

### Table 777 (Compounds (I.C-Ar.1-1) to (I.C-Ar.1-282))

Compounds of formula (I.C), wherein R⁵ is a radical (Ar.1) as defined in line 1 of table B and R¹, R² and R³ have one of the meanings corresponding to any line of table A.

### Tables 778 to 898 (Compounds (I.C-Ar.2-1) to (I.C-Ar.122-282))

Compounds of formula (I.C), wherein R⁵ is one of the radicals (Ar.2) to (Ar.122) as defined in lines 2 to 122 of table B and R¹, R² and R³ have one of the meanings corresponding to any line of table A.

### Table 899 (Compounds (I.C-Het.1-1) to (I.C-Het.1-282))

Compounds of formula (I.C), wherein R⁵ is a radical (Het.1) as defined in line 1 of table C and R¹, R² and R³ have one of the meanings corresponding to any line of table A.

### Tables 900 to 1164 (Compounds (I.C-Het.2-1) to (I.C-Het.266-282))

Compounds of formula (I.C), wherein R⁵ is one of the radicals (Het.2) to (Het.266) as defined in lines 2 to 266 of table C and R¹, R² and R³ have one of the meanings corresponding to any line of table A.

Yet another particular embodiment of the invention relates to sulfonamide compounds of formula (I), wherein m is 0, X is O, Y is N and Z is O, in the following referred to as sulfonamide compounds of formula (I.D)

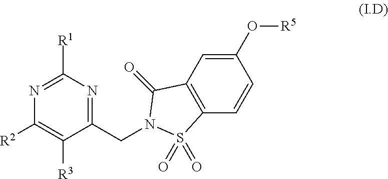

wherein R¹, R², R³ and R⁵ have the meanings given above, especially those meanings mentioned as being preferred.

### Table 1165 (Compounds (I.D-Ar.1-1) to (I.D-Ar.1-282))

Compounds of formula (I.D), wherein R⁵ is a radical (Ar.1) as defined in line 1 of table B and R¹, R² and R³ have one of the meanings corresponding to any line of table A.

### Tables 1166 to 1286 (Compounds (I.D-Ar.2-1) to (I.D-Ar.122-282))

Compounds of formula (I.D), wherein R⁵ is one of the radicals (Ar.2) to (Ar.122) as defined in lines 2 to 122 of table B and R¹, R² and R³ have one of the meanings corresponding to any line of table A.

### Table 1287 (Compounds (I.D-Het.1-1) to (I.D-Het.1-282))

Compounds of formula (I.D), wherein R⁵ is a radical (Het.1) as defined in line 1 of table C and R¹, R² and R³ have one of the meanings corresponding to any line of table A.

### Tables 1288 to 1552 (Compounds (I.D-Het.2-1) to (I.D-Het.266-282))

Compounds of formula (I.D), wherein R⁵ is one of the radicals (Het.2) to (Het.266) as defined in lines 2 to 266 of table C and R¹, R² and R³ have one of the meanings corresponding to any line of table A.

The compounds of the formula (I) according to the present invention can be prepared by various routes in analogy to prior art processes known per se for preparing biphenyl compounds and processes known per se for preparing sulfimide compounds like saccharin derivatives. Advantageously, they can be obtained as outlined in schemes 1 to 5.

Generally, sulfonamid compounds of formula (I) can be prepared by reaction of a compound of formula (II) with a boronic acid derivative of the formula R⁵—(Z)—B(OR^{1a})(OR^{1b}) by a Suzuki coupling as shown in scheme 1.

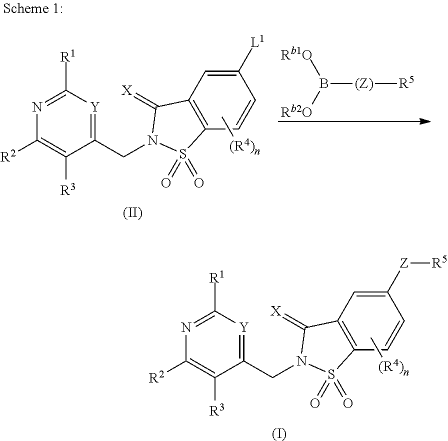

In scheme 1, R¹, R², R³, R⁴, R⁵, X, Y, Z and n are as defined above, R^{b1}, and R^{b2} are each independently hydrogen or C₁-C₄-alkyl, or R^{b1}, and R^{b2} together form an 1,2-ethylene moiety the carbon atoms of which may be unsubstituted or may all or in part be substituted by methyl groups, and L¹ is a suitable leaving group. Suitable leaving groups L¹ are halogen, preferably chlorine, bromine or iodine, alkylcarboxylate, benzoate, alkylsulfonate, haloalkylsulfonate or arylsulfonate, most preferably chlorine or bromine.

The reaction is usually carried out in the presence of a base and a catalyst, in particular a palladium catalyst, such as for example described in the following literature: Synth. Commun. Vol. 11, p. 513 (1981); Acc. Chem. Res. Vol. 15, pp. 178-184 (1982); Chem. Rev. Vol. 95, pp. 2457-2483 (1995); Organic Letters Vol. 6 (16), p. 2808 (2004); “Metal catalyzed cross coupling reactions”, 2nd Edition, Wiley, VCH 2005 (Eds. De Meijere, Diederich); “Handbook of organopalladium chemistry for organic synthesis” (Ed. Negishi), Wiley, Interscience, New York, 2002; “Handbook of functionalized organometallics”, (Ed. P. Knochel), Wiley, VCH, 2005.

Suitable catalysts are in tetrakis(triphenylphosphine)palladium(0); bis(triphenylphosphine)palladium(II) chloride; bis(acetonitrile)palladium(II) chloride; [1,1′-bis(diphenylphosphino)ferrocene]-palladium(II) chloride/methylene chloride (1:1) complex; bis[bis-(1,2-diphenylphosphino)ethane]palladium(0); bis(bis(1,2 diphenylphosphino)butane]-palladium(II) chloride; palladium(II) acetate; palladium(II) chloride; and palladium(II) acetate/tri-o-tolylphosphine complex or mixtures of phosphines and Pd salts or phosphines and Pd-complexes e.g. dibenzylideneacetone-palladium and tri-tert-butylphosphine (or its tetrafluoroborate), triscyclohexylphosphine; or a polymer-bound Pd-triphenylphosphine catalyst system.

Suitable bases are, in general, inorganic compounds, such as alkali metal and alkaline earth metal oxides, such as lithium oxide, sodium oxide, calcium oxide and magnesium oxide, alkali metal and alkaline earth metal carbonates, such as lithium carbonate, sodium carbonate, potassium carbonate, cesium carbonate and calcium carbonate, and also alkali metal bicarbonates, such as sodium bicarbonate, alkali metal and alkaline earth metal alkoxides, such as sodium methoxide, sodium ethoxide, potassium ethoxide and potassium tert.-butoxide, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to bases such as sodium carbonate, potassium carbonate, caesium carbonate, triethylamine and sodium bicarbonate.

The base is used in a molar ratio in the range of 1:1 to 1:10, preferably a 1:1.5 to 1:5 relative to the compounds (II), the boronic acid is used in a molar ratio in the range of 1:1 to 1:5, preferably 1:1 to 1:2.5 relative to the compounds (II). In some cases it may be beneficial for easy purification to use the boronic acid in a substoechi-ometric molar ratio in the range of 0.7:1 to 0.99:1, relative to the compounds (II).

The reaction is usually carried out in an inert organic solvent. Suitable solvents are aliphatic hydrocarbons, such as pentane, hexane, cyclohexane and petroleum ether, aromatic hydrocarbons, such as toluene, o-, m- and p-xylene, ethers, such as diisopropyl ether, tert.-butyl methyl ether, dioxane, anisole and tetrahydrofuran and dimethoxyethane, ketones, such as acetone, methyl ethyl ketone, diethyl ketone and tert.butyl methyl ketone, and also dimethyl sulfoxide, dimethylformamide and dimethylacetamide, particularly preferably ethers, such as tetrahydrofuran, dioxane and dimethoxyethane. It is also possible to use mixtures of the solvents mentioned, or mixtures with water.

The reaction is usually carried out at temperatures of from 20° C. to 180° C., preferably from 40° C. to 120° C.

After completion of the reaction, the compounds of formula (I) can be isolated by employing conventional methods such as adding the reaction mixture to water, extracting with an organic solvent, concentrating the extract an the like. The isolated compounds (I) can be purified by a technique such as chromatography, recrystallization and the like, if necessary.

It is also possible to add a scavenger to the reaction mixtures to remove byproducts or unreacted starting materials by binding to those and simple filtration. For details see “Synthesis and purification catalog”, Argonaut, 2003 and literature cited therein.

Boronic acids or esters of formula R⁵—(Z)—B(OR^{1a})(OR^{1b}) are commercially available or can be prepared according to “Science of Synthesis” Vol. 6, Thieme, 2005;

WO 02/042275; Synlett 2003, (8) p. 1204; J. Org. Chem., 2003, 68, p. 3729, Synthesis, 2000, p. 442, J. Org. Chem., 1995, 60, p. 750; or “Handbook of functionalized organometallics”, (Ed. P. Knochel), Wiley, VCH, 2005.

Compounds (II), wherein X is O or NH, can be obtained by intramolecular cyclisation of sulfonamide compounds of formula (V) as shown in scheme 2.

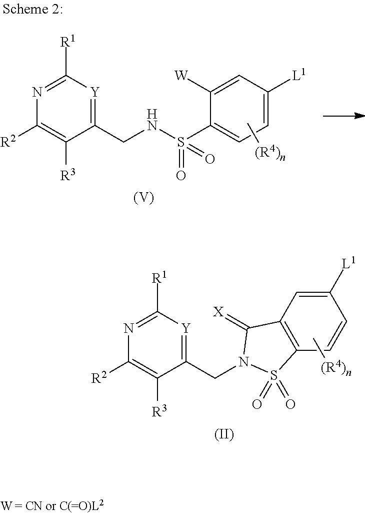

In scheme 2, L¹, R¹, R², R³, R⁴, Y and n are as defined above, and W is CN or —C(═O)L², wherein L² is a leaving group such as halogen or alkoxy, preferably C₁-C₄-alkoxy.

The cyclisation of the sulfonamide (V) to the iminosaccharin (II), wherein X is NH, can be performed according to the method outlined in U.S. Pat. No. 5,981,758. The reaction is usually carried out in an inert organic solvent in the presence of a base. Suitable solvents are aliphatic hydrocarbons, such as pentane, hexane, cyclohexane and petroleum ether, aromatic hydrocarbons, such as toluene, o-, m- and p-xylene, ethers, such as diisopropyl ether, tert.-butyl methyl ether, dioxane, anisole and tetrahydrofuran and dimethoxyethane, ketones, such as acetone, methyl ethyl ketone, diethyl ketone and tert.butyl methyl ketone, and also dimethyl sulfoxide, dimethylformamide and dimethylacetamide, It is also possible to use mixtures of the solvents mentioned, or mixtures with water. Preference is given to using ethers, such as tetrahydrofuran, dioxane and dimethoxyethane or aromatic hydrocarbons, such as toluene as a mixture with water.

The reaction is usually carried out at temperatures of from 0° C. to 80° C., preferably from 10° C. to 25° C.

Suitable bases are, in general, inorganic compounds, such as alkali metal and alkaline earth metal oxides, such as lithium oxide, sodium oxide, calcium oxide and magnesium oxide, alkali metal and alkaline earth metal carbonates, such as lithium carbonate, sodium carbonate, potassium carbonate, cesium carbonate and calcium carbonate, and also alkali metal bicarbonates, such as sodium bicarbonate, alkali metal and alkaline earth metal alkoxides, such as sodium methoxide, sodium ethoxide, potassium ethoxide and potassium tert.-butoxide, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to bases such as sodium carbonate, potassium carbonate, caesium carbonate, triethylamine and sodium bicarbonate.

Iminosaccharin compounds of formula (II), wherein X is NH, may subsequently be converted into saccharin compounds of formula (II), wherein X is O by hydrolysis (cf. preparation example 1.4), or into iminosaccharin compounds of formula (II), wherein X is NR^{x}, wherein R^{x} is different from hydrogen, by reaction with an alkylating or acylating agent of formula R^{x}-LG, wherein LG is a suitable leaving group, such as halogen, alkylsulfonate or arylsulfonate.

Acid hydrolysis of iminosaccharin compounds of formula (II) is carried out according to the method outlined in U.S. Pat. No. 5,981,758. A variety of acids can be used for the hydrolysis for example but not limited to hydrochloric acid, hydrobromic acid, triflic acid, trifluoroacidic acid, acetic acid and methanesulfonic acid. Suitable solvents include both aqueous and organic solvents. A preferred combination of acid and solvent is hydrochloric acid in aqueous dioxane.

Compounds (V) can be obtained by reaction of a sulfonylchlorides (III) with an amino compound (IV) as shown in scheme 3.

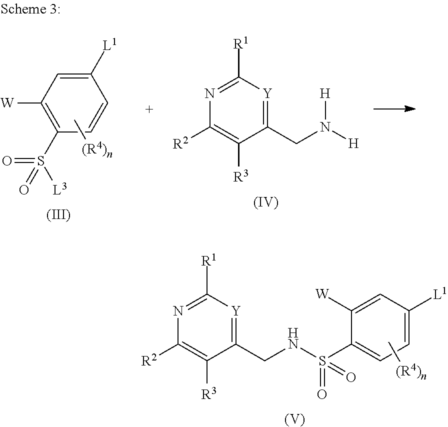

In scheme 3, L¹, L², R¹, R², R³, R⁴, W, Y and n are as defined above, and L³ is a leaving group such as hydroxy or halogen, preferably chlorine.

The reaction of a sulfonyl compound (III) with an amino compound (IV) can be performed in accordance with standard methods of organic chemistry, see for example, Lieb. Ann. Chem. p. 641, 1990, or WO 2005/033081.

This reaction is usually carried out in an inert organic solvent. Suitable solvents are aliphatic hydrocarbons, such as pentane, hexane, cyclohexane and petroleum ether, aromatic hydrocarbons, such as toluene, o-, m- and p-xylene, halogenated hydrocarbons, such as dichloromethane, chloroform and chlorobenzene, ethers, such as diethyl ether, diisopropyl ether, tert.-butyl-methyl-ether, dioxane, anisole and tetrahydrofuran, nitriles, such as acetonitrile and propionitrile, ketones, such as acetone, methyl ethyl ketone, diethyl ketone and tert.-butyl-methyl-ketone, and also dimethyl sulfoxide, dimethylformamide and dimethylacetamide, preferably tetrahydrofuran, methyl tert-butyl ether, methylene chloride, chloroform, acetonitrile, toluene or dimethylformamide. It is also possible to use mixtures of the solvents mentioned.

It may be advantageous to carry out the reaction in the presence of a base. Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, disopropylethylamine and N-methylpiperidine, pyridine, substituted pyridines, such as collidine, lutidine and 4-dimethylamino [symbol] pyridine, and also bicyclic amines. Particular preference is given to pyridine, triethylamine and potassium carbonate. The bases are generally employed in equimolar amounts, in excess or, if appropriate, as solvent. The excess of base is typically 0.5 to 5 moles relative to 1 mole of compounds (IV).

Generally, the reaction is carried out at temperatures of from −30° C. to 120° C., preferably from −10° C. to 100° C.

The starting materials are generally reacted with one another in equimolar amounts.

If the sulfonyl compounds (III) are not commercially available, they can be obtained according to procedures known in the art, e.g. suitable compounds of formula (III) can be obtained by replacement of an amino group of suitable amino precursors by sulfonylation (cf. preparation example I.1.1).

The amino compounds (IV) are known from the literature or are commercially available, or they can be prepared from precursors (VIa) to (VIc) wherein V is as defined in scheme 4 by reduction.

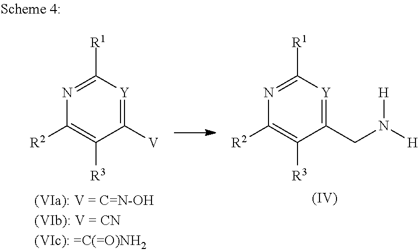

Methods of this reduction can be found in the literature e.g. in Houben-Weyl, Band 10/4, Thieme, Stuttgart, 1968; Band 11/2, 1957; Band E5, 1985; J. Heterocycl. Chem., 1997, 34 (6), pp. 1661-1667; J. Chem. Soc. 1954, p. 1165; Heterocycles, 41(4), pp. 675-688, 1995; J. Org. Chem., 1982, 47, p. 3153; Heterocycles, 1996, 43 (9), pp. 1893-1900; J. Prakt. Chem-Chem. Ztg. 336(8), pp. 695-697, 1994; or are known to those skilled in the art.

The oxims (VIa) can be prepared from either the respective aldehyd (V═CHO; compounds (VId)) or the methylderivative (V═CH₃; compounds (VIe)), as described in Houben-Weyl, Band 10/4, Thieme, Stuttgart, 1968; Band 11/2, 1957; Band E5, 1985; J. Prakt. Chem-Chem. Ztg. 336(8), pp. 695-697, 1994; Tetrahedron Lett. 42(39), pp. 6815-6818, 2001; or Heterocycles, 29(9), pp. 1741-1760, 1989.

The aldehyds (VId) are commercially available (e.g. pyridine-4-carboxaldehyde, quinolin-4-carboxaldehyd, 2-chloropyridine-4-carboxaldehyd) or can be synthesized from a 4 methylpyridines or 4-methylpyrimidines in analogy to the method described in J. Org. Chem. 51(4), pp. 536-537, 1986, or from a haloderivative (V=halogen, compounds (VIf)) as shown in Eur. J. Org. Chem., 2003, (8), pp. 1576-1588; Tetrahedron Lett. 1999, 40 (19), pp. 3719-3722; Tetrahedron, 1999, 55 (41), pp. 12149-12156.

The methyl derivatives (Vie) are commercially available (e.g. 4-methylpyridine, 3-cyano-2,6-dichloro-4-methylpyridine, 4-methylquinoline, 6-chloro 4 methylquinoline, 6,8-dimethoxy-quinoline, 4-methylpyrimidin, 4,6-dimethylpyrimidine) or can be synthesized in analogy to “Science of Synthesis”, Vol 15, Thieme, Stuttgart, 2005.

The nitriles (VIb) can be prepared either from the respective halogen derivative (VIf) (V=halogen, preferably chlorine, bromine or iodine,) by reaction with a cyanide source with or without additional catalysts, as described e.g. in Tetrahedron Lett. 42(38), pp. 6707-6710, 2001; Chem. Eur. J., 2003, 9 (8), pp. 1828-1836; Chem. Commun. (Cambridge), 2004, (12), pp. 1388-1389; J. Organomet. Chem. 2004, 689 (24), pp. 4576-4583; or J. Chem. Soc. Perk. T., 1 (16), pp. 2323-2326, 1999. Alternatively, the amide or oxime may be dehydrated to the corresponding nitrile (VIb) as outlined in “Synthesis”, Stuttgart, (10), pp. 943-944, 1992; or literature cited therein; or Heterocycl. Chem. 1997, 34 (6), pp. 1661-1667.

The 4-halogene quinolines (compounds VIf, wherein Y is CR^{y} and R² together with R³ form a fused benzene ring) are either commercially available or can be synthesized according to “Science of Synthesis”, Vol 15, Thieme, Stuttgart, 2005 or e.g. according to the following literature or citations therein: 4-chloro-6,7-dimethoxy-quinoline: Journal Med. Chem. 48(5), p. 1359, 2005; 4-chloro-5,7-dichloro-quinoline: Indian, 187817, 29 June 2002; 4-chloro-7-chloro-quinoline: Tetrahedron, 60 (13), p. 3017, 2004; 4-chloro-7-trifluoromethyl-quinoline; Tetrahedron Lett., 31(8), p. 1093, 1990; 4-chloro-7,8-dimethoxy-quinoline: Tetrahedron, 41 (15), p. 3033, 1985; 4-chloro-8 methoxy quinoline: Chem. Berichte 118(4), p. 1556, 1985; 4-chloro-(6 or 7 or 8)-iodo quinoline, 4-bromo-(6 or 7 or 8)-iodo-quinoline, 4-iodo-(6 or 7 or 8)-iodo-quinoline: J. Med. Chem., 21(3), p. 268, 1978.

Further methods to build up appropriate precursors or modify substitution pattern can be found in “Synthesis”, Stuttgart (1), pp. 31-32, 1993; Tetrahedron, 1993, 49 (24), pp. 5315-5326; “Methods in Science of Synthesis”, Band 15, and literature cited therein; Bioorg. Med. Chem. Lett. 1997, 7 (23), pp. 2935-2940; J. Am. Chem. Soc., 1946, 68, p. 1264; or Org. Synth. 1955, III, p. 272.

In some cases it can be beneficial in terms of ease of work up or purification to perform the reduction of compounds (VI) to compounds (IV) and the reaction of the amine (IV) with the compound (III) in one pot without isolating compounds (IV).

Alternatively, compounds of formula (I) may be obtained by first reacting a compound of formula (V) with a boronic acide derivative R⁵—(Z)—B(OR^{b1})(OR^{b2}) and subsequently submitting the obtained coupling product (VII) to an intramolecular cyclisation as shown in scheme 5.

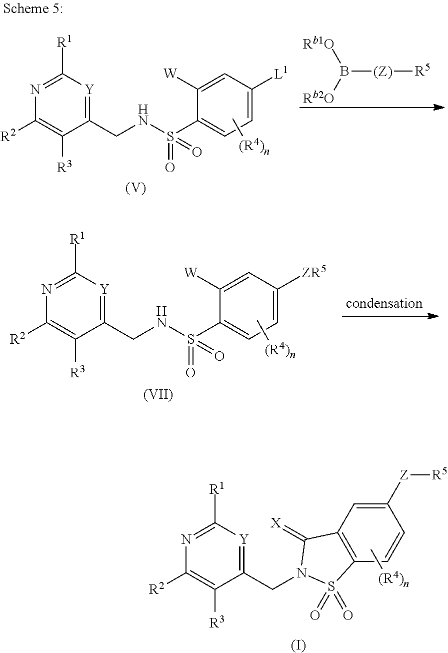

The conditions for this reaction sequence correspond to the conditions outlined for the Suzuki coupling and the intramolecular cyclisation before.

In some cases it can be beneficial in terms of ease of work up or purification to perform the coupling of compounds (V) with the boronic acid derivative to compounds (VII) and the subsequent intramolecular cyclisation of compound (VII) to compounds (I) in one pot without isolating compounds (VII).

Alternatively, compounds of formula (I) may be obtained by reacting a pyridine or pyrimidine compound of formula (VIII) with a saccharine or iminosaccharine derivative of formula (IX) as shown in scheme 6.

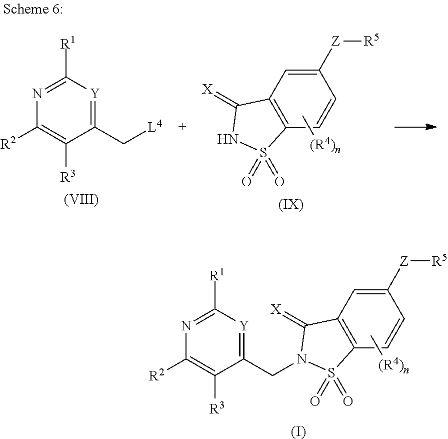

In scheme 6, R¹, R², R³, R⁴, R⁵, X, Y, Z and n are as defined above, and L⁴ is a suitable leaving group, such as halogen, alkylsulfonate or arylsulfonate.

If individual compounds (I) are not obtainable by the routes described above, they can be prepared by derivatization of other compounds (I) or by customary modifications of the synthesis routes described.

The preparation of the compounds of formula (I) may lead to them being obtained as isomer mixtures (stereoisomers, enantiomers). If desired, these can be resolved by the methods customary for this purpose, such as crystallization or chromatography, also on optically active adsorbate, to give the pure isomers.

The N-oxides may be prepared from the compounds (I) according to conventional oxidation methods, for example by treating a compound (I) with an organic peracid such as metachloroperbenzoic acid [Journal of Medicinal Chemistry, 38(11), 1892-1903 (1995); WO 03/64572] or with inorganic oxidizing agents such as hydrogen peroxide, [see Journal of Heterocyclic Chemistry, 18(7), 1305-8 (1981)] or oxone, see Journal of the American Chemical Society, 123(25), 5962-5973 (2001).

Accordingly, one aspect of the present invention relates to a process for the preparation of sulfonamide compounds of formula (I), as defined above, comprising, reacting a compound of formula (II), as defined above, with a boronic acid derivative of the formula R⁵—(Z)—B(OR^{b1})(OR^{b2}) in the presence of a base and a transition metal catalyst to give sulfonamid compounds of formula (I). In one embodiment this process for the preparation of sulfonamide compounds of formula (I) additionally comprises, reacting a sulfonyl compound of formula (III), as defined above, with a amino compound of formula (IV), as defined above, in the presence of a base to yield a compound of the formula (V), as defined above, which is subsequently submitted to an intramolecular cyclysation to give a compound of formula (II), as defined above.

Another aspect of the present invention relates to a process for the preparation of sulfonamide compounds of formula (I), as defined above, comprising, submitting the obtained compound (VII) to an intramolecular cyclisation to give a sulfonamid compound of formula (I), as defined above. In one embodiment this process for the preparation of sulfonamide compounds of formula (I) additionally comprises, reacting a compound of formula (V), as defined above, with a boronic acid derivative of the formula R⁵—(Z)—B(OR^{b1})(OR^{b2}), as defined above, in the presence of a base and a transition metal catalyst to give sulfonamid compounds of formula (VII), as defined above.

Another aspect of the present invention relates to a process for the preparation of sulfonamide compounds of formula (I), as defined above, comprising, reacting a pyridine or pyrimidine compound of formula (VIII) with a saccharine or iminosaccharine derivative of formula (IX), as shown in scheme 6.

The intermediates of formula (II) are novel. Therefore, another aspect of the invention relates to sulfonamide compounds of formula (II),

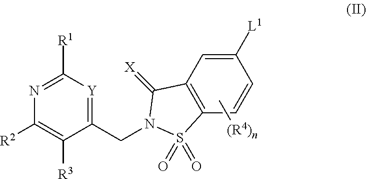

wherein L¹, R¹, R², R³, R⁴, n, X and Y have one of the meanings as defined for the compounds of formula (I) before and wherein L¹ is a suitable leaving group, such as halogen, preferably chlorine, bromine or iodine, alkylcarboxylate, benzoate, alkylsulfonate, haloalkylsulfonate or arylsulfonate. Preferably L¹ is halogen, especially chlorine or bromine.

The remarks made before concerning preferred embodiments of the variables of the compounds of formula (I) apply accordingly for the variables of the compounds of formula (II).

Due to their excellent activity, the compounds of formula (I) may be used for controlling animal pests.

Accordingly, the present invention also provides a method for controlling animal pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula (I) or a salt or an N-oxide thereof or a composition as defined herein.

In a particular embodiment, the method of the invention serves for protecting plant propagation material (such as seed) and the plant which grows therefrom from animal pest attack or infestation and comprises treating the plant propagation material (such as seed) with a pesticidally effective amount of a compound of formula (I) or an agriculturally acceptable salt or an N-oxide thereof as defined above or with a pesticidally effective amount of an agricultural composition as defined above and below. The method of the invention is not limited to the protection of the “substrate” (plant, plant propagation materials, soil material etc.) which has been treated according to the invention, but also has a preventive effect, thus, for example, according protection to a plant which grows from a treated plant propagation materials (such as seed), the plant itself not having been treated.

In the sense of the present invention, “animal pests” are preferably selected from invertebrate pests, in particular from arthropods and nematodes, more preferably from harmful insects, arachnids and nematodes, and even more preferably from insects. In another embodiment of the invention, “animal pests” are selected from rodents and other vertebrate pests, except for humans, such as.

The invention further provides an agricultural composition for combating such animal pests, which comprises such an amount of at least one compound of formula (I) or at least one agriculturally useful salt thereof or an N-oxide thereof and at least one inert liquid and/or solid agronomically acceptable carrier that has a pesticidal action and, if desired, at least one surfactant.

Such a composition may contain a single active compound of formula (I) or a salt or an N-oxide thereof or a mixture of several active compounds of formula (I) or their salts or their N-oxides according to the present invention. The composition according to the present invention may comprise an individual isomer or mixtures of isomers as well as individual tautomers or mixtures of tautomers.

The compounds of the formula (I), their salts and their N-oxides and the pestidicidal compositions comprising them are effective agents for controlling invertebrate pests, in particular arthropod pests and nematodes. Animal pests controlled by the compounds of formula (I) include for example:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lamb-dina fiscellaria, Laphygma exigua, Leucoptera coffeella, Leucoptera scitella, Lithocol-letis blancardella, Lobesia botrana, Loxostege sticticalis, Lymantria dispar, Lymantria monacha, Lyonetia clerkella, Malacosoma neustria, Mamestra brassicae, Orgyia pseu-dotsugata, Ostrinia nubilalis, Panolis flammea, Pectinophora gossypiella, Peridroma saucia, Phalera bucephala, Phthorimaea operculella, Phyllocnistis citrella, Pieris bras-sicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12 punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria;
dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa;
thrips (Thysanoptera), e.g. Dichromothrips corbetti, Frankliniella fusca, Frankliniella occidentalis, Frankliniella tritici, Scirtothrips citri, Thrips oryzae, Thrips palmi and Thrips tabaci;
hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta;
heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor;
homopterans (Homoptera), e.g. Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus persicae, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Sogatella furcifera Trialeurodes vaporariorum, Toxoptera aurantiiand, and Viteus vitifolii;
termites (Isoptera), e.g. Calotermes flavicollis, Leucotermes flavipes, Reticulitermes flavipes, Reticulitermes lucifugus and Termes natalensis;
orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;
Arachnoidea, such as arachnids (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis;
Siphonatera, e.g. Xenopsylla cheopsis, Ceratophyllus spp.

The compositions and compounds of formula (I) their salts and their N-oxides are also useful for the control of nematodes, especially plant parasitic nematodes such as root knot nematodes, Meloidogyne hapla, Meloidogyne incognita, Meloidogyne javanica, and other Meloidogyne species;

cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nema-todes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Paratylen-chus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nema-todes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compositions and compounds of formula (I) their salts and their N-oxides are also useful for the control of harmfull vertebrates, except for humans, in particular rodents (order Rodentia), including

rodents from the families Muridae, in particular Murinae, Cricetidae and Myocastoridae; especially rodents of the genera Rattus, Mus, Microtus, Apodemus, Arvicola and Clethrionomys, in particular the species Rattus norvegicus, Rattus rattus, Rattus argentiventer, Rattus exulans, Mus sp. Arvicola terrestris, Microtus arvalis, Microtus pennsylvanicus, Tatera indica, Peromyscus leucopus, Peromyscus maniculatus, Mastomys natalensis, Sigmodon hispidus, Arvicanthis niloticus, Bandicota bengalensis, Bandicota indica, Nesokia indica, Meriones hurrinanae and Millardia meltada. Very especially attention is to be given to the representatives of the genera Rattus and Mus, for example R. rattus, R. norvegicus, M. musculus, and nutria (Myocastor coypus).

In addition, the compositions and compounds of formula (I) their salts and their N-oxides are also useful for the control of are also suitable for controlling other harmful vertebrates, except for human beings and pets, for example vertebrate pests of the order didelphimorphia, in particular didelphidae such as opossums (vulpes vulpes), and American opossums (Didelphidae), brushtail possums (Trichosurus), in particular the common brushtail possum (Trichosurus vulpecula), of the order lagomorpha, in particular of the family leporidae, such as rabbit (i.e. suitable genera from the subfamily Leporinae) and of the family procyonidae, such as raccoons, in particular Procyon Cofor.

The invention therefore also relates to a method of controlling vertebrate pests, in particular rodent pests, wherein a bait formulation according to the invention is applied in the habitat of the harmful vertebrates.

In a preferred embodiment of the invention the compounds of formula (I) are used for controlling insects or arachnids, in particular insects of the orders Lepidoptera, Coleoptera, Thysanoptera and Homoptera and arachnids of the order Acarina. The compounds of the formula (I) according to the present invention are particularly useful for controlling insects of the order Thysanoptera and Homoptera.

The compounds of formula (I) or the pesticidal compositions comprising them may be used to protect growing plants and crops from attack or infestation by animal pests, especially insects, acaridae or arachnids by contacting the plant/crop with a pesticidally effective amount of compounds of formula (I). The term “crop” refers both to growing and harvested crops.

The compounds of formula (I) can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents (liquid carriers) and/or solid carriers, if desired, surfactants such as emulsifiers and dispersants, preservatives, anti-foaming agents, anti-freezing agents, for seed treatment formulation and bait formulation also optionally colorants and/or binders and/or gelling agents.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (N-methylpyrrolidone [NMP], N-octylpyrrolidone [NOP]), acetates (glycol diacetate), glycols, fatty acid dimethylamides, fatty acids and fatty acid esters. In principle, solvent mixtures may also be used.

Suitable emulsifiers are in particular non-ionic and anionic emulsifiers.

Examples of dispersants are lignin sulfonates, lignin-sulfite waste liquors, synthetic polymers such as polyacrylates, polyvinylpyrrolidone, etc. and cellulose derivatives such as methylcellulose.

Suitable surfactants include the aforementioned dispersants and emulsifiers such alkali metal, alkaline earth metal and ammonium salts of lignosulfonic acid, naphthalenesulfonic acid, phenolsulfonic acid, dibutylnaphthalene-sulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of sulfonated phenols with formaldehyde, condensates of naphthalene or of naphthalenesulfonic acid with phenol derivatives and formaldehyde, polyethoxylated phenols such as polyoxyethylene octylphenol ether, ethoxylated isooctylphenol, ethoxylated octylphenol, ethoxylated nonylphenol, alkylphenol polyglycol ethers, tributylphenyl polyglycol ether, tristearylphenyl polyglycol ether (ethoxylated tristyrylphenol), alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

A suitable preservative is e.g. dichlorophen.

Seed treatment formulations may additionally comprise binders and optionally colorants.

Binders can be added to improve the adhesion of the active materials on the seeds after treatment. Suitable binders are block copolymers EO/PO surfactants but also polyvinylalcoholsl, polyvinylpyrrolidones, polyacrylates, polymethacrylates, polybute-nes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®).

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers.

Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compounds of formula (I) can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations:

1. Products for dilution with water for foliar applications. For seed treatment purposes, such products may be applied to the seed diluted or undiluted.

### A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formula-tion with 10% (w/w) of active compound(s) is obtained.

### B) Dispersible Concentrates (DC)

20 parts by weight of the active compound(s) are dissolved in 70 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

### C) Emulsifiable Concentrates (EC)

15 parts by weight of the active compound(s) are dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

### D) Emulsions (EW, EO, ES)

25 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

### E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

### F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

### G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

### H) Gel-Formulation (GF)

In an agitated ball mill, 20 parts by weight of the active compound(s) are comminuted with addition of 10 parts by weight of dispersants, 1 part by weight of a gelling agent wetters and 70 parts by weight of water or of an organic solvent to give a fine active compound(s) suspension. Dilution with water gives a stable suspension of the active compound(s), whereby a formulation with 20% (w/w) of active compound(s) is obtained.

2. Products to be applied undiluted for foliar applications. For seed treatment purposes, such products may be applied to the seed diluted or undiluted.

I) Dustable powders (DP, DS)

5 parts by weight of the active compound(s) are ground finely and mixed intimately with 95 parts by weight of finely divided kaolin. This gives a dustable product having 5% (w/w) of active compound(s)

### J) Granules (GR, FG, GG, MG)

0.5 parts by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

### K) ULV Solutions (UL)

10 parts by weight of the active compound(s) are dissolved in 90 parts by weight of an organic solvent, for example xylene. This gives a product having 10% (w/w) of active compound(s), which is applied undiluted for foliar use.

The compounds of formula (I) are also suitable for the treatment of plant propagation materials (such as seed). Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having preger-minated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Other preferred FS formulations of compounds of formula (I) for seed treatment comprise from 0.5 to 80 wt % of the active ingredient, from 0.05 to 5 wt % of a wetter, from 0.5 to 15 wt % of a dispersing agent, from 0.1 to 5 wt % of a thickener, from 5 to 20 wt % of an anti-freeze agent, from 0.1 to 2 wt % of an anti-foam agent, from 1 to 20 wt % of a pigment and/or a dye, from 0 to 15 wt % of a sticker/adhesion agent, from 0 to 75 wt % of a filler/vehicle, and from 0.01 to 1 wt % of a preservative.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula (I) are effective through both contact (via soil, glass, wall, bed net, carpet, plant parts or animal parts), and ingestion (bait, or plant part).

For use against ants, termites, wasps, flies, mosquitos, crickets, or cockroaches, compounds of formula (I) are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula (I) as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula (I) and their respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula (I) and their respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and diethylenically unsaturated hydrocarbons, such as styrene, and aliphatic diens, such as butadiene.

The impregnation of curtains and bednets is done in general by dipping the textile material into emulsions or dispersions of the active compounds of formula (I) or spraying them onto the nets.

Methods which can be employed for treating the seed are, in principle, all suitable seed treatment and especially seed dressing techniques known in the art, such as seed coating (e.g. seed pelleting), seed dusting and seed imbibition (e.g. seed soaking). Here, “seed treatment” refers to all methods that bring seeds and the compounds of formula (I) into contact with each other, and “seed dressing” to methods of seed treatment which provide the seeds with an amount of the compounds of formula (I), i.e. which generate a seed comprising the compound of formula (I). In principle, the treatment can be applied to the seed at any time from the harvest of the seed to the sowing of the seed. The seed can be treated immediately before, or during, the planting of the seed, for example using the “planter's box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of formula (I) or the N-oxides or veterinarily acceptable salts thereof are in particular also suitable for being used for combating parasites in and on animals.

A further object of the present invention is therefore to provide new methods for controlling parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula (I) or the N-oxides or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula (I) or the N-oxides or veterinarily acceptable salts thereof or a composition comprising it.

The invention also provides a process for the preparation of a composition for treating, controlling, preventing or protecting animals against infestation or infection by parasites which comprises a parasiticidally effective amount of a compound of formula (I) or the N-oxides or veterinarily acceptable salts thereof or a composition comprising it.

Activity of compounds against agricultural pests does not suggest their suitability for control of endo- and ectoparasites in and on animals which requires, for example, low, nonemetic dosages in the case of oral application, metabolic compatibility with the animal, low toxicity, and a safe handling.

Surprisingly, it has been found that compounds of formula (I) are suitable for combating endo- and ectoparasites in and on animals.

Compounds of formula (I) or the N-oxides or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula (I) or the N-oxides or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula (I) or the N-oxides or veterinarily acceptable salts thereof and compositions comprising them are suitable for systemic and/or non-systemic control of ecto- and/or endoparasites. They are active against all or some stages of development.

The compounds of formula (I) are especially useful for combating ectoparasites.

The compounds of formula (I) are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,
cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,
actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,
bugs (Heteropterida): Cimex lectularius, Cimex hemipterus, Reduvius senilis, Triatoma spp., Rhodnius ssp., Panstrongylus ssp. and Arilus critatus,
Anoplurida, e.g. Haematopinus spp., Linognathus spp., Pediculus spp., Phtirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,
Rhabditida, e.g. Rhabditis spp, Strongyloides spp., Helicephalobus spp, Strongylida, e.g. Strongylus spp., Ancylostoma spp., Necator americanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,
Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula (I) and compositions containing them are particularly useful for the control of pests from the orders Diptera, Siphonaptera and Ixodida.

Moreover, the use of compounds of formula (I) and compositions containing them for combating mosquitoes is especially preferred.

The use of the compounds of formula (I) and compositions containing them for combating flies is a further preferred embodiment of the present invention.

Furthermore, the use of the compounds of formula (I) and compositions containing them for combating fleas is especially preferred.

The use of the compounds of formula (I) and of the compositions containing them for combating ticks is a further preferred embodiment of the present invention.

The compounds of formula (I) also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the compounds of formula (I) may be formulated as animal feeds, animal feed premixes, animal feed concentrates, pills, solutions, pastes, suspensions, drenches, gels, tablets, boluses and capsules. In addition, the compounds of formulae I may be administered to the animals in their drinking water. For oral administration, the dosage form chosen should provide the animal with 0.01 mg/kg to 100 mg/kg of animal body weight per day of the compounds of formeulae I and II, preferably with 0.5 mg/kg to 100 mg/kg of animal body weight per day.

Alternatively, the compounds of formula (I) may be administered to animals parenterally, for example, by intraruminal, intramuscular, intravenous or subcutaneous injection. The compounds of formula (I) may be dispersed or dissolved in a physiologically acceptable carrier for subcutaneous injection. Alternatively, the compounds of formula (I) may be formulated into an implant for subcutaneous administration. In addition the compounds of formula (I) may be transdermally administered to animals. For parenteral administration, the dosage form chosen should provide the animal with 0.01 mg/kg to 100 mg/kg of animal body weight per day of the compounds of formula (I).

The compounds of formula (I) may also be applied topically to the animals in the form of dips, dusts, powders, collars, medallions, sprays, shampoos, spot-on and pour-on formulations and in ointments or oil-in-water or water-in-oil emulsions. For topical applica-tion, dips and sprays usually contain 0.5 ppm to 5 000 ppm and preferably 1 ppm to 3 000 ppm of the compounds of formula (I). In addition, the compounds of formula (I) may be formulated as ear tags for animals, particularly quadrupeds such as cattle and sheep.

Suitable preparations are:

Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methylpyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester. Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents are water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N methylpyrrolidone, 2-pyrrolidone, 2,2-dimethyl-4-oxy-methylene-1,3-dioxolane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyldodecanol, cetylstearyl alcohol, oleyl alcohol, and fatty acids such as oleic acid and mixtures thereof.

Suitable hydrophilic phases are: water, alcohols such as propylene glycol, glycerol, sorbitol and mixtures thereof.

Suitable emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether; ampholytic surfactants such as di-sodium N-lauryl-p-iminodipropionate or lecithin; anionic surfactants, such as sodium lauryl sulfate, fatty alcohol ether sulfates, mono/dialkyl polyglycol ether orthophosphoric acid ester monoethanolamine salt; cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of applica-tion, and the like.

The compositions which can be used in the invention can comprise generally from about 0.001 to 95% of the compounds of formula (I).

Generally, it is favorable to apply the compounds of formula (I) in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula (I) against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula (I) are applied dermally/topically.

In a further preferred embodiment, the topical application is conducted in the form of compound-containing shaped articles such as collars, medallions, ear tags, bands for fixing at body parts, and adhesive strips and foils.

Generally, it is favorable to apply solid formulations which release compounds of formula (I) in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula (I). A detailed list of plastics and elastomers as well as preparation procedures for the shaped articles is given e.g. in WO 03/086075.

Compositions of the invention which can be applied against rodents and other harmful vertebrate pests, include in particular bait formulations but also seed treatment formulations, as treated seed may itself serve as a bait.

Bait formulations include, besides at least one compound of the formula (I), a salt or an N-oxide thereof at least one bait material and optionally further ingredients such as attractants, pain killers, biocides, adjuvants and/or further formulation additives which are typically for bait formulations. Suitable bait formulations have been principally described e.g. in DE 2506769, EP 317260, U.S. Pat. No. 4,190,734, GB 1053088, GB 1274442, DE 4444261, WO 98/04129, WO 01/80645, WO 2003/094612, WO 2007/057393, WO 2007/031796 and WO 2009/047175 etc and the literature cited therein.

The total amount of the compound of the formula (I), the salt or the N-oxide thereof will be generally in the range from 0.001 to 50% by weight, based on the total weight of the formulation, the remainder including at least one bait material and optionally further ingredients as mentioned herein.

Bait materials which are generally used are vegetable or animal foodstuffs and feed stuffs. Suitable examples are coarse cereal meals, cereal grains, flaked cereals or cereal meals (for example of oats, wheat, barley, maize, soya, rice), flaked coconut, ground coconut, sugar syrups (for example obtained by hydrolyzing starch (glucose syrup), invert sugar syrup, beet sugar syrup, maple syrup), sugars (for example sucrose, lactose, fructose, glucose), grated nuts, ground nuts (for example hazelnut, walnut, almond), vegetable fat/oils (for example rapeseed oil, soya fat, sunflower oil, cocoa butter, peanut oil, peanut butter, corn oil), animal fats/oils (butter, lard, fish oil), proteins (for example dried skimmed milk, dried egg, protein hydrolysates) and minerals (for example common salt).

Preferred are vegetable foodstuffs such as oatmeal, flaked oats, wheat kernels, coarse wheat meals, wheat flour, maize meal, flaked coconut, ground coconut, glucose syrup, maple syrup, beet sugar syrup, sucrose, glucose, ground hazelnuts, ground walnuts, almond, rapeseed oil, soya fat, peanut oil, corn oil; animal fats such as butter; proteins such as, for example, dried egg and dried skimmed milk.

Especially preferred are vegetable foodstuffs such as oatmeal, maize meal, flaked coconut, ground coconut, glucose syrup, maple syrup, sucrose, ground hazelnuts, soya fat, peanut oil, peanut butter and proteins such as dried skimmed milk.

For the purposes of the invention, an attractant is a substance (or substance mixture) which is a phagostimulant or which attracts the attention of the rodent pest to the bait without being a feedstuff proper in another way, in particular by odor (for example as a sexual attractant). Examples of attractants are pheromones, yeast, ground crustaceans, fecal matter, berries, chocolate, fish meal, meat, black pepper and flavor enhancers such as glutamates, in particular sodium glutamate and disodium glutamate.

The amount of bait material in the formulation may vary depending on the formulation type. The amount of bait material will generally be in the range from 1 to 99.99% by weight, based on the weight of the formulation.

Examples of bactericides include thiazolinones, such as Proxel® from ICI or Acticide® RS from Thor Chemie, Kathon® MK from Rohm & Haas and Dowicil® from Dow Elanco.

Pain killers include analgesics and sedatives as well as mixtures of analgesics and sedatives. Examples of analgesics include morphine, codeine, dihydrocodeine, hydro-morphine, oxycodone, pethidine, tramadol, methadone, acetylsalicylic acid, diflunisal, naproxen, proxicam, tenoxicam, meloxicam, paracetamol and phenazone. Examples of sedatives include propofol, clonidine, barbiturates such as phenobarbital and pentobarbital and benzodiazepines, in particular those which have been mentioned among the anxiolytics. Examples of mixtures of analgesics and sedatives are mixtures comprising one or more analgesics from the group consisting of morphine, codeine, dihydrocodeine, hydromorphine, oxycodone, pethidine, tramadol, methadone, acetylsalicylic acid, diflunisal, naproxen, proxicam, tenoxicam, meloxicam, paracetamol and phenazone and one or more sedatives from the group consisting of propotol, clonidine, phenobarbital, pentobarbital, alprazolam, bromazepam, brotizolam, chlordiazepoxide, clobazam, clonazepam, diazepam, clorazepate, flunitrazepam, flurazepam, loprazolam, lorazepam, lormetazepam, medazepam, midazolam, nitrazepam, nordazepam, oxazepam, prazepam, temazepam, tetrazepam and triazolam.

Further customary formulation additives comprise colorants, bittering agents, flow agents, binders, agents for improving weather resistance, and antioxidants.

Colorants are frequently added, and the bait formulation is thereby clearly logged as not for consumption, in order to avoid ingestion by mistake by humans or non-target animals. In particular, blue colorants serve to deter birds. However, they may also serve to detect the consumption of the bait in the rodent pests' feces or vomit.

Bittering agents serve to avoid incidental consumption by humans. Especially preferred is denatonium benzoate, which, in a suitable concentration (in general 1 to 200 ppm, in particular 5 to 20 ppm), has a most unpleasant taste for humans, but not for rodents.

Flow agents and binders are added as a function of the bait formulation type. Binders are capable of fixing the mixture according to the invention onto the surface of the bait component (for example cereal grains) or—in the case of pastes for example—impart structure and coherence. Flow agents such as mineral earths and aluminosilicates facilitate extrudation and they are therefore frequently used in pellets and extruded blocks.

Suitable agents for improving weather resistance are, for example, paraffin waxes.

Examples of suitable antioxidants are t-butylhydroquinone (TBHQ), butylated hydroxytoluenes and butylated hydroxyanisoles, preferably in an amount of from 10 ppm to 20 000 ppm.

Preferred bait formulations are food baits, in particular seed cereal baits and suitable treatment agents, pellets (die-formed articles), wax-coated pellets, molten-wax blocks, compressed or extruded wax blocks, pastes, gels, granules and foams.

Feed baits frequently consist of cereal which may be present in different fine forms, for example in the form of grains or else as a more or less finely ground meal.

The advantage of a bait in the form of a meal is that it is difficult to carry away and store by the animals, but is not easy to handle (dust) and spoils rapidly.

The disadvantage of intact grains is that they may also be ingested by non-target animals, for example birds, but under certain circumstances also humans.

The compounds of the present invention and optionally further adjuvants may be applied to the surface of the feedstuffs, in particular in conjunction with binders. Preferred is therefore the treatment of such feedstuffs with at least one compound according to the invention in the manner of a seed treatment. Thus, by analogy to WO 2007/057393, a suitable formulation for treating seed for preparing a seed based bait may comprise
(a) at least one compound of the formula (I), or a salt or N-oxide thereof, optionally in combination with at least one pain killer;
(b) at least one polyol;
(c) an adhesive, and, if appropriate,
(d) a monosaccharide and/or a disaccharide and/or an oligosaccharide.

In such compositions the polyol content may be from 1% by weight to 50% by weight, preferably 1 to 20% by weight, based on the weight of the composition.

The adhesive content may be from 1% by weight to 30% by weight, preferably from 1 to 10% by weight, especially preferably from 1 to 5% by weight, based on the weight of the composition.

The monosaccharide and/or disaccharide and/or oligosaccharide content may be from 10% by weight to 50.0% by weight, preferably from 10.0 to 35.0% by weight, especially preferably from 15.0 to 25.0% by weight, based on the weight of the composition.

Suitable polyols include glycol, polyethylene glycol, glycerol, propylene glycol, dipropylene glycol, preferably glycerol.

As a component c) mono- and disaccharides are preferred. The use of disaccharides is very especially preferred. Suitable monosaccharides are glucose, fructose, galactose, preferably fructose. Suitable disaccharides are sucrose, maltose, lactose, preferably sucrose (for example in pure form or as molasses, beet sugar). A suitable oligosaccharide is starch.

Suitable adhesives are ethylene oxide/propylene oxide copolymers, polyvinyl alcohol (for example Mowiol®4-98, Clariant, Rhodoviol®60-20, Rhone-Poulence), polyvinylpyrrolidone (Sokalan® HP 50, BASF, Kollidon® 25, BASF, Luvitec® K80, BASF Agrimer® A, ISP Global Techn), polyacrylates (for example Sokalan® PA 110 S, BASF), polymethyl methacrylates, water-soluble polyolefin derivatives such as polybutene derivatives, polyethylene oxides (for example polyethers) or polyisobutyl derivatives (for example copolymers of polyolefins and maleic anhydride derivatives (for example Densodrin BA® by BASF), polystyrene derivatives (for example copolymers of styrene and maleic anhydride derivatives or copolymers of styrene and acrylic acid derivatives, or styrene/butadiene-based latex copolymers, obtainable, for example, under the name Semkote E-125, Uniqema) and polyethyleneamines, polyethyleneamides, polyethyleneimines (for example Lupasol® BASF, Polymin®, BASF), polyurethanes (Semkote E-105, Uniqema), polyvinyl acetate, tylose of and polyethylene wax (for example commercially available under the name Poligen® WE 7 BASF, preferably ethylene oxide/propylene oxide copolymers, polyacrylates (for example Sokalan® PA 110 S, BASF), polymethyl methacrylates, water-soluble polyolefin derivatives such as polybutene derivatives, polyethylene oxides (for example polyethers) or polyisobutyl derivatives (for example copolymers of polyolefins and maleic anhydride derivatives (for example Densodrin BA® by BASF), polystyrene derivatives (for example copolymers of styrene and maleic anhydride derivatives or copolymers of styrene and acrylic acid derivatives, or styrene/butadiene-based latex copolymers, obtainable, for example, under the name Semkote E-125, Uniqema), polyethyleneamines, polyethyleneamides, polyethyleneimines (for example Lupasol® BASF, Polymin®, BASF), polyurethanes (Semkote E-105, Uniqema), polyvinyl acetate, and polyethylene wax (for example commercially available under the name Poligen® WE 7 BASF); especially preferably, ethylene oxide/propylene oxide copolymers, polyacrylates (for example Sokalan® PA 110 S, BASF), polymethyl methacrylates, polystyrene derivatives (for example copolymers of styrene and maleic anhydride derivatives or copolymers of styrene and acrylic acid derivatives, or styrene/butadiene-based latex copolymers, obtainable, for example, under the name Semkote E-125, Uniqema) and polyethylene wax (for example commercially available under the name Poligen® WE 7 BASF);

Moreover, the bait formulations according to the invention may optionally comprise yet further adjuvants such as, for example, surfactants (such as wetting agents, adhesives and dispersants), antifoams, thickeners and colorants as mentioned before.

The treatment can be carried out by methods known to the skilled worker (for example by spraying or immersing/incubating the cereal grains in, or with, a formulation according to the invention, if appropriate using a suitable device such as a continuously or batchwise-operating seed dresser). In doing so, the formulation may preferably be diluted with up to 7.5 g of water/kg of cereal grains. The treated grains may optionally be dried.

Furthermore preferred as bait formulation are what are known as pellets (die-formed articles). Such pellets comprise a compound of the formula (I), a salt or an N-oxide of (I) according to the invention in a mixture with optionally powdered or ground feedstuffs (B), in particular cereal and thickeners, and other formulation additives (C). Pellets are usually prepared by compressing, extrusion and subsequent drying.

The pellet size varies as a function of the target animals. Frequently, pellets are prepared in the form of cylinders of diameter 3 to 5 mm and a length of 5 to 10 mm.

In general, the content of the compounds according to the invention is from 0.001 to 30% by weight of the pellets.

To increase the weather resistance of pellets, paraffin wax is added in one embodiment of the invention, which, however, reduces the palatability of the pellets for the rodent pests.

A further preferred bait formulation are wax block formulations which, in addition to the at least one compound of the formula (I) a salt or an N-oxide thereof, comprise a mixture of feedstuffs, typically cereal grains, coarse cereal meals or cereal powders (B), if appropriate formulation additives (C) and paraffin wax. Wax block formulations have the advantage that their weather resistance is good; however, at the expense of the palatability for the rodent pests. Wax block formulations are usually prepared by casting, extruding or compressing, the wax content in the last-mentioned methods being lower, which—with a similarly good weather resistance—increases palatability. Wax block formulations may be prepared in many shapes which allow them for example to be hung up or fastened in a bait station. In a preferred embodiment, the wax blocks comprise a multiplicity of corners because the animals prefer to gnaw at corners.

A further preferred bait formulation are granules which, in addition to the at least one compound of the formula (I) a salt or an N-oxide thereof, comprise a typically comminuted, for example ground feedstuff (R) and, if appropriate, further additives, and a binder. The preparation of granules is described for example in EP-A 0 771 393.

A furthermore preferred bait formulation are gels (see, for example, WO 03/094612 and the literature cited therein). Preferably, such gels comprise
water as dispersant;
at least one thickener;
at least one compound according to the present invention;
one or more feedstuffs;

In a preferred embodiment, the gels additionally comprise one or more of the following components:
base;
humectant;
oxidation stabilizers;
colorants;
bittering agents;
further additives.

Thickeners which are used are organic and inorganic macromolecules. Organic macromolecules which may be mentioned are cellulose derivatives, for example hydroxypropylcellulose, hydroxyethylcellulose, methylcellulose, carboxymethylcellulose-sodium, hydroxypropylmethylcellulose, hydroxyethylmethylcellulose, hydroxyethylpropylcellulose such as xanthans, alginates, carrageenan, agar-agar, polyvinyl alcohols, polyvinylpyrrolidone, polyacrylic acid and polymethacrylic acid. Inorganic macromolecules (inorganic gel formers) which may be mentioned is highly-dispersed silica and bentonites (for example Rudolf Voigt, Pharmazeutische Technologie [Pharmaceutical technology], pages 362 to 385, Ulstein Mosby).

Bases which are employed are, for example, alkali metal hydroxides, alkaline earth metal hydroxides and amino derivatives, such as potassium hydroxide, sodium hydroxide, triethanolamine or ammonium hydroxide solution. Bases may be employed for adjusting the pH.

Humectants which are employed are, for example, polyols such as glycerol, propylene glycol, ethylene glycol, sugar alcohols and polypropylene glycols. Humectants prevent the gel from drying out so that it remains elastic and is not scattered when taken up by the rodents. Preferred humectants are glycerol, propylene glycol, polypropylene glycol 200, propylene glycol 300, polypropylene glycol 400, sorbitol, mannitol and xylitol.

Oxidation stabilizers which may be mentioned are butylhydroxytoluene, butylhydroxyanisole, tocopherols (for example vitamin E) or ascorbic acid and its derivatives (for example ascorbic acid palmitate, sodium ascorbate). The foodstuffs and feedstuffs can be stabilized by adding oxidation stabilizers. Especially preferred are butylhydroxytoluene, vitamin E, ascorbic acid.

Colorants which may be mentioned are blue, green and red pigments and blue, green and red soluble dyes (see Colour Index, Fourth Edition: Hue blue, red, green), with blue colorants being preferred for deterring birds. A gel according to the invention may be colored by colorants for warning purposes.

Suitable colorants which are also approved for coloring cosmetics are preferred (cf., for example, Otterstatter, Die Farbung von Lebensmitteln, Arzneimitteln, Kosmetika [Coloring foodstuffs, pharmaceuticals, cosmetics], Behr's Verlag, 2nd edition, pages 52 to 57). It is preferred to use pigmentsas colorants.

Additives which may be used are, for example, bittering agents such as denatonium benzoate and natural and synthetic aroma chemicals (see, for example, product list of Hamann & Reimer, Holzminden).

A further preferred type of bait formulation are high-resistant foams or flexible foams, preferably flexible foams. High-resistant foams which can be employed in accordance with the invention are described for example in FR-PS 2 676 888 and U.S. Pat. No. 4,190,734. Flexible foams which can be employed in accordance with the invention are described for example in GB-PS 1 053 088 and GB 1 274 442.

Preferred foams are the flexible foams described in DE-A 44 44 261. Such flexible foams comprise
at least one compound according to the present invention;
at least one hydrophilic polymer having an average molecular weight of from 2000 to 60 000 (determined by gel permeation chromatography (GPC)), which is preferably selected from the series of the long-chain polyurethanes, polyesters, polyester polyols, polystyrenes, polybutadienes, maleic acid polymers, each of which is modified in the polymer chain by carboxyl and/or amino groups,
long-chain aliphatic C₆-C₂₂-fatty acids such as palmitic acid, dodecanoic acid and stearic acid, or their alkali metal, alkaline earth metal and ammonium salts,
and, if appropriate, further adjuvants from the series consisting of colorants, emulsifiers, solvents, attractants and feedstuffs.

The hydrophilic polymers are known in the art and have been described for example in H. Kittel, Lehrbuch der Lacke and Beschichtungen [Textbook of paints and coatings], Volume IV, pages 76 to 306, Verlag W. A. Colomb (1096) or in the same textbook, edition (1976), Volume IV, pages 328 to 358 as binders for paints.

Hydrophilic polymers which may be used in flexible foams are physically drying binders, for example those whose binders are based on a fully reacted, linear polyurethane of (i) a polyester polyol, (ii) a chain extender, (iii) a diisocyanate and (iv) a hydroxycarboxylic acid. Suitable polyester polyols (i) for the preparation of such polyurethanes are, for example, adipic acid, alkanediol, polyester diols of the molecular weight range from 600 to 3000 (number average). The alkanediols are, for example, butane-1,4-diol, hexane-1,6-diol, neopentyl glycol or mixtures of such glycols. Suitable chain extenders (ii) are, for example, diols of the type employed for the preparation of the polyester diols, and also diamines such as hexamethylenediamine or isophoronediamine. Examples of suitable diisocyanates (iii) are 4,4′-diphenylmethane diisocyanate, isophorone diisocyanate or hexamethylene diisocyanate. The polyurethanes are prepared in the manner known per se by reacting the starting materials, with an equivalent ratio of isocyanate groups to isocyanate groups of reactive groups of from 0.9:1 to 1.1:1 being maintained.

Oxidatively drying binders may also be used. Such binders which may be mentioned are those based on polybutadiene, styrene and maleic anhydride and having ionic groups, as they are described in the applications EP 170184 and EP 270795.

The hydrophilic polymers generally have an average molecular weight of from 2000 to 60 000 g/mol, preferably of from 2500 to 25 000 g/mol (number average). They are present in the finished formulation in a concentration of from 2.5 to 40, preferably 2.5 to 10, % by weight based on the weight of the total formulation.

Generally, the foam formulations are premixes. As a rule, they will be diluted with water in amounts of from 0 to 80% before being applied.

The flexible foams can be prepared in a manner known per se by stirring or shaking. Another possibility is the preparation in situ during application, using blowing agents.

Blowing agents for the preparation of the formulations according to the invention which may be mentioned are CO₂, N₂O, lower alkanes such as propane or n-butane, isobutane, halogen-containing lower alkanes and low-boiling ethers such as dimethyl ether, and mixtures of said blowing agents.

Suitable bait formulation may also comprises a particulate mixture which, besides particles comprising a feed stuff and the at least one compound according to the invention, also comprises particles which comprise a feedstuff with a flavor which differs from that of the first feedstuff and which differ from the first-mentioned particles in terms of size, shape, surface texture, internal texture, color, density and/or content. For example, the non-rodenticidal particles are present in an amount of from 2.5 to 10% by weight (based on the total formulation). The non-rodenticidal particles are preferably based on cereals and preferably comprise at least one further attractant from the group consisting of chocolate, dried and ground crustaceans, yeast, fecal matter, fish meal, meat and berries. Examples of such particulate formulations are described in WO 2007/031796.

The invention therefore also relates to a method of controlling rodent pests, wherein a bait formulation according to the invention is applied in the habitat of the harmful vertebrates.

The invention furthermore relates to the use of a bait formulation according to the invention for controlling rodent pests.

The bait formulation are suitable for application in rooms, for example cellars, stores, pantries, animal houses, or in gutters and in the open, for example in runs of the rodent pests, or in holes in which they dwell.

The bait formulation according of the invention is applied in a bait box. Such bait boxes are described for example in U.S. Pat. No. 3,750,326, U.S. Pat. No. 4,349,982, DE-A 195 01 892, WO 02/102147, DE-A 10 2004 022 105 and DE-A 10 2004 022 103.

Compositions to be used according to this invention may also contain other active ingredients, for example other pesticides, insecticides, herbicides, fungicides, other pesticides, or bactericides, fertilizers such as ammonium nitrate, urea, potash, and super-phosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents can be admixed with the agents used according to the invention in a weight ratio of 1:10 to 10:1. Mixing the compounds of formula (I) or the compositions comprising them in the use form as pesticides with other pesticides frequently results in a broader pesticidal spectrum of action.

The following list M of pesticides together with which the compounds according to the invention can be used and with which potential synergistic effects might be produced, is intended to illustrate the possible combinations, but not to impose any limitation:

M.1. Organo(thio)phosphates: acephate, azamethiphos, azinphos-ethyl, azinphos-methyl, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicro-tophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirim-fos, temephos, terbufos, tetrachlorvinphos, thiometon, triazophos, trichlorfon, vamidothion;
M.2. Carbamates: aldicarb, alanycarb, bendiocarb, benfuracarb, butocarboxim, butoxy-carboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate;
M.3. Pyrethroids: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-, yfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flu-cythrinate, flumethrin, tau-fluvalinate, halfenprox, imiprothrin, metofluthrin, permethrin, phenothrin, prallethrin, profluthrin, pyrethrin (pyrethrum), resmethrin, silafluofen, tefluthrin, tetramethrin, tralomethrin, transfluthrin;
M.4. Juvenile hormone mimics: hydroprene, kinoprene, methoprene, fenoxycarb, pyriproxyfen;
M.5. Nicotinic receptor agonists/antagonists compounds: acetamiprid, bensultap, car-tap hydrochloride, clothianidin, dinotefuran, imidacloprid, thiamethoxam, nitenpyram, nicotine, spinosad (allosteric agonist), spinetoram (allosteric agonist), thiacloprid, thio-cyclam, thiosultap-sodium and AKD1022;
M.6. GABA gated chloride channel antagonist compounds: chlordane, endosulfan, gamma-HCH (lindane); acetoprole, ethiprole, fipronil, pyrafluprole, pyriprole, vaniliprole;
M.7. Chloride channel activators: abamectin, emamectin benzoate, milbemectin, le-pimectin;
M.8. METI I compounds: fenazaquin, fenpyroximate, pyrimidifen, pyridaben, tebufen-pyrad, tolfenpyrad, flufenerim, rotenone;
M.9. METI II and III compounds: acequinocyl, fluacyprim, hydramethylnon;
M.10. Uncouplers of oxidative phosphorylation: chlorfenapyr, DNOC;
M.11. Inhibitors of oxidative phosphorylation: azocyclotin, cyhexatin, diafenthiuron, fenbutatin oxide, propargite, tetradifon;
M.12. Moulting disruptors: cyromazine, chromafenozide, halofenozide, methoxy-fenozide, tebufenozide;
M.13. Synergists: piperonyl butoxide, tribufos;
M.14. Sodium channel blocker compounds: indoxacarb, metaflumizone;
M.15. Fumigants: methyl bromide, chloropicrin sulfuryl fluoride;
M.16. Selective feeding blockers: crylotie, pymetrozine, flonicamid;
M.17. Mite growth inhibitors: clofentezine, hexythiazox, etoxazole;
M.18. Chitin synthesis inhibitors: buprofezin, bistrifluoron, chlorfluazuron, diflubenzuron, flucycloxuron, flufenoxuron, hexaflumuron, lufenuron, novaluron, noviflumuron, te-flubenzuron, triflumuron;
M.19. Lipid biosynthesis inhibitors: spirodiclofen, spiromesifen, spirotetramat;
M.20. Octapaminergic agonists: amitraz;
M.21. Ryanodine receptor modulators: flubendiamide, (R)—,(S)-3-Chlor-N1-{2-methyl-4-[1,2,2,2-tetrafluor-1-(trifluormethyl)ethyl]phenyl}-N2-(1-methyl-2-methylsulfonylethyl)phthalamid (M21.1);
M.22. Various: aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, organic sulfur compounds, tartar emetic, sulfoxaflor, 4-But-2-ynyloxy-6-(3,5-dimethyl-piperidin-1-yl)-2-fluoro-pyrimidine (M22.1), 3-Benzoylamino-N-[2,6-dimethyl-4-(1,2,2,2-tetrafluoro-1-trifluoromethyl-ethyl)-phenyl]-2-fluoro-benzamide (M22.2), 4-[5-(3,5-Dichloro-phenyl)-5-trifluoromethyl-4,5-dihydro-isoxazol-3-yl]-2-methyl-N-pyridin-2-ylmethyl-benzamide (M22.3), 4-[5-(3,5-Dichloro-phenyl)-5-trifluoromethyl-4,5-dihydro-isoxazol-3-yl]-2-methyl-N-(2,2,2-trifluoro-ethyl)-benzamide (M22.4), 4-[5-(3,5-Dichloro-phenyl)-5-trifluoromethyl-4,5-dihydro-isoxazol-3-yl]-2-methyl-N-thiazol-2-ylmethyl-benzamide (M22.5), 4-[5-(3,5-Dichloro-phenyl)-5-trifluoromethyl-4,5-dihydro-isoxazol-3-yl]-2-methyl-N-(tetrahydro-furan-2-ylmethyl)-benzamide (M22.6), 4-{[(6-Bromopyrid-3-yl)methyl](2-fluoroethyl)amino}furan-2(5H)-on (M22.7), 4-{[(6-Fluoropyrid-3-yl)methyl](2,2-difluoroethyl)amino}furan-2(5H)-on (M22.8), 4-{[(2-Chloro1,3-thiazolo-5-yl)methyl](2-fluoroethyl)amino}furan-2(5H)-on (M22.9), 4-{[(6-Chloropyrid-3-yl)methyl](2-fluoroethyl)amino}furan-2(5H)-on (M22.10), 4-{[(6-Chloropyrid-3-yl)methyl](2,2-difluoroethyl)amino}furan-2(5H)-on (M22.11), 4-{[(6-Chloro-5-fluoropyrid-3-yl)methyl](methyl)amino}furan-2(5H)-on (M22.12), 4-{[(5,6-Dichloropyrid-3-yl)methyl](2-fluoroethyl)amino}furan-2(5H)-on (M22.13), 4-{[(6-Chloro-5-fluoropyrid-3-yl)methyl](cyclopropyl)amino}furan-2(5H)-on (M22.14), 4-{[(6-Chloropyrid-3-yl)methyl](cyclopropyl)amino}furan-2(5H)-on (M22.15), 4-{[(6-Chloropyrid-3-yl)methyl](methyl)amino}furan-2(5H)-on (M22.16), Cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-[2-hydroxy-4,6a,12b-trimethyl-11-oxo-9-(3-pyridinyl)-2H,11H-naphtho[2,1-b]pyrano[3,4-e]pyran-3,6-diyl]ester (M22.17), 8-(2-Cyclopropylmethoxy-4-methyl-phenoxy)-3-(6-methyl-pyridazin-3-yl)-3-azabicyclo[3.2.1]octane (M22.18);
M.23. N—R′-2,2-dihalo-1-R″cyclo-propanecarboxamide-2-(2,6-dichloro-α,α,α-tri-fluoro-ptolyl)hydrazone or N—R′-2,2-di(R′″)propionamide-2-(2,6-dichloro-α,α,α-trifluoro-p-tolyl)hydrazone, wherein R′ is methyl or ethyl, halo is chloro or bromo, R″ is hydrogen or methyl and R′″ is methyl or ethyl;
M.24. Anthranilamides: chloranthraniliprole, cyantraniliprole;
5-Bromo-2-(3-chloro-pyridin-2-yl)-2H-pyrazole-3-carboxylic acid [4-cyano-2-(1-cyclopropyl-ethylcarbamoyl)-6-methyl-phenyl]-amide (M24.1),
5-Bromo-2-(3-chloro-pyridin-2-yl)-2H-pyrazole-3-carboxylic acid [2-chloro-4-cyano-6-(1-cyclopropyl-ethylcarbamoyl)-phenyl]-amide (M24.2),
5-Bromo-2-(3-chloro-pyridin-2-yl)-2H-pyrazole-3-carboxylic acid [2-bromo-4-cyano-6-(1-cyclopropyl-ethylcarbamoyl)-phenyl]-amide (M24.3),
5-Bromo-2-(3-chloro-pyridin-2-yl)-2H-pyrazole-3-carboxylic acid [2-bromo-4-chloro-6-(1-cyclopropyl-ethylcarbamoyl)-phenyl]-amide (M24.4),
5-Bromo-2-(3-chloro-pyridin-2-yl)-2H-pyrazole-3-carboxylic acid [2,4-dichloro-6-(1-cyclopropyl-ethylcarbamoyl)-phenyl]-amide (M24.5),
5-Bromo-2-(3-chloro-pyridin-2-yl)-2H-pyrazole-3-carboxylic acid [4-chloro-2-(1-cyclopropyl-ethylcarbamoyl)-6-methyl-phenyl]-amide (M24.6),
   M.25. Malononitrile compounds: CF₂HCF₂CF₂CF₂CH₂C(CN)₂CH₂CH₂CF₃, (2-(2,2,3,3,4,4,5,5-octafluoropentyl)-2-(3,3,3-trifluoro-propyl)malononitrile), CF₃(CH₂)₂C(CN)₂CH₂(CF₂)₅CF₂H, (2-(2,2,3,3,4,4,5,5,6,6,7,7-Dodecafluoroheptyl)-2-(3,3,3-trifluoro-propyl)-malononitrile), CF₃(CH₂)₂C(CN)₂(CH₂)₂C(CF₃)₂F (2-(3,4,4,4-Tetrafluoro-3-trifluoromethyl-butyl)-2-(3,3,3-trifluoro-propyl)-malononitrile), CF₃(CH₂)₂C(CN)₂(CH₂)₂(CF₂)₃CF₃ (2-(3,3,4,4,5,5,6,6,6-Nonafluoro-hexyl)-2-(3,3,3-trifluoro-propyl)-malononitrile), CF₂H(CF₂)₃CH₂C(CN)₂CH₂(CF₂)₃CF₂H (2,2-Bis(2,2,3,3,4,4,5,5-octafluoro-pentyl)-malononitrile), CF₃(CH₂)₂C(CN)₂CH₂(CF₂)₃CF₃ (2-(2,2,3,3,4,4,5,5,5-Nonafluoro-pentyl)-2-(3,3,3-trifluoro-propyl)-malononitrile), CF₃(CF₂)₂CH₂C(CN)₂CH₂(CF₂)₃CF₂H (2-(2,2,3,3,4,4,4-Heptafluoro-butyl)-2-(2,2,3,3,4,4,5,5-octafluoro-pentyl)-malononitrile), CF₃CF₂CH₂C(CN)₂CH₂(CF₂)₃CF₂H (2-(2,2,3,3,4,4,5,5-Octafluoro-pentyl)-2-(2,2,3,3,3-pentafluoro-propyl)-malononitrile), CF₂HCF₂CF₂CF₂CH₂C(CN)₂CH₂CH₂CF₂CF₃ (2-(2,2,3,3,4,4,5,5-octafluoropentyl)-2-(3,3,4,4,4-pentafluorobutyl)-malonodinitrile), CF₃(CH₂)₂C(CN)₂CH₂(CF₂)₃CF₂H (2-(2,2,3,3,4,4,5,5-octafluoropentyl)-2-(3,3,3-trifluoro-butyl)-malononitrile);
   M.26. Microbial disruptors: Bacillus thuringiensis subsp. Israelensi, Bacillus sphaericus, Bacillus thuringiensis subsp. Aizawai, Bacillus thuringiensis subsp. Kurstaki, Bacillus thuringiensis subsp. Tenebrionis;

The commercially available compounds of the group M may be found in The Pesticide Manual, 13th Edition, British Crop Protection Council (2003) among other publications.

Thioamides of formula M6.1 and their preparation have been described in WO 98/28279. Lepimectin is known from Agro Project, PJB Publications Ltd, November 2004. Benclothiaz and its preparation have been described in EP-A1454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1 462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. AKD 1022 and its preparation have been described in U.S. Pat. No. 6,300,348. Chloranthraniliprole has been described in WO 01/70671, WO 03/015519 and WO 05/118552. Anthranilamide derivatives of formula M24.1 have been described in WO 01/70671, WO 04/067528 and WO 05/118552. Cyflumetofen and its preparation have been described in WO 04/080180. The aminoquinazolinone compound pyrifluquinazon has been described in EP A 109 7932. Sulfoximine sulfoxaflor has been described in WO 2006/060029 and WO 2007/149134. The alkynylether compound M22.1 is described e.g. in JP 2006131529. Organic sulfur compounds have been described in WO 2007060839. The carboxamide compound M 22.2 is known from WO 2007/83394. The oxazoline compounds M 22.3 to M 22.6 have been described in WO 2007/074789. The furanon compounds M 22.7 to M 22.16 have been described eg. in WO 2007/115644. The pyripyropene derivative M 22.17 has been described in WO 2008/66153 and WO 2008/108491. The pyridazin compound M 22.18 has been described in JP 2008/115155. The malononitrile compounds have been described in WO 02/089579, WO 02/090320, WO 02/090321, WO 04/006677, WO 05/068423, WO 05/068432 and WO 05/063694.

Fungicidal mixing partners are those selected from the group consisting of acylalanines such as benalaxyl, metalaxyl, ofurace, oxadixyl, amine derivatives such as aldimorph, dodine, dodemorph, fenpropimorph, fenpropidin, guazatine, iminoctadine, spiroxamin, tridemorph, anilinopyrimidines such as pyrimethanil, mepanipyrim or cyrodinyl, antibiotics such as cycloheximid, griseofulvin, kasugamycin, natamycin, polyoxin or streptomycin, azoles such as bitertanol, bromoconazole, cyproconazole, difenoconazole, dini-cona-zole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol, dicarboximides such as iprodion, myclozolin, procymidon, vinclozolin, dithiocarbamates such as ferbam, nabam, maneb, mancozeb, metam, metiram, propineb, polycarbamate, thiram, ziram, zineb, heterocyclic compounds such as anilazine, benomyl, bosca-lid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine, copper fungicides such as Bordeaux mixture, copper acetate, copper oxychloride, ba-sic copper sulfate, nitrophenyl derivatives such as binapacryl, dinocap, dinobuton, nitrophthalisopropyl, phenylpyrroles such as fenpiclonil or fludioxonil, sulfur, other fungicides such as acibenzolar-S-methyl, benthiavalicarb, carpropamid, chlorothalonil, cyflufenamid, cymoxanil, diclomezin, diclocymet, diethofencarb, edifen-phos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid, strobilurins such as azoxystrobin, dimoxystrobin, fluoxastrobin, kresoxim-methyl, me-tominostrobin, orysastrobin, picoxystrobin or trifloxystrobin, sulfenic acid derivatives such as captafol, captan, dichlofluanid, folpet, tolylfluanid, cinnemamides and analogs such as dimethomorph, flumetover or flumorph.

The animal pest, i.e. arthropodes and nematodes, the plant, soil or water in which the plant is growing can be contacted with the present compound(s) of formula (I) or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

Moreover, animal pests may be controlled by contacting the target pest, its food supply, habitat, breeding ground or its locus with a pesticidally effective amount of compounds of formula (I). As such, the application may be carried out before or after the infection of the locus, growing crops, or harvested crops by the pest.

“Locus” means a habitat, breeding ground, cultivated plants, plant propagation material (such as seed), soil, area, material or environment in which a pest or parasite is growing or may grow.

In general “pesticidally effective amount” means the amount of active, ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula (I) and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of are applied not only to the surrounding soil surface or into the under-floor soil in order to protect wooden materials but it can also be applied to lumbered articles such as surfaces of the under-floor concrete, alcove posts, beams, plywood, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of formula (I) can also be applied preventively to places at which occurrence of the pests is expected.

The compounds of formula (I) may also be used to protect growing plants from attack or infestation by pests by contacting the plant with a pesticidally effective amount of compounds of formula (I). As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95% by weight, preferably from 0.1 to 45% by weight, and more preferably from 1 to 25% by weight of at least one repellent and/or insecticide.

For use in bait compositions, the typical content of active ingredient is from 0.001% by weight to 15% by weight, desirably from 0.001% by weight to 5% by weight of active compound.

For use in spray compositions, the content of active ingredient is from 0.001 to 80% by weight, preferably from 0.01 to 50% by weight and most preferably from 0.01 to 15% by weight.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the active ingredients are generally from 0.1 g to 10 kg per 100 kg of seed, preferably from 0.1 g to 5 kg per 100 kg of seed, in particular from 1 g to 200 g per 100 kg of seed.

The present invention is now illustrated in further detail by the following examples.

### I. Experimental Procedures

With due modification of the starting compounds, the protocols shown in the synthesis example below were used for obtaining further compounds of formula (I).

The products were characterized by coupled High Performance Liquid Chromatography/mass spectrometry (HPLC/MS), by ¹H-NMR (400 MHz) in CDCl₃ or d₆-DMSO or by their melting points. HPLC column: RP-18 column (Chromolith Speed ROD from Merck KgaA, Germany). Elution: acetonitrile+0.1% trifluoroacetic acid (TFA)/water in a ratio of from 5:95 to 95:5 in 5 minutes at 40° C. MS: Quadrupol electrospray ionisation, 80 V (positiv modus).

### I.1 Preparation of 5-(2,4-bis(trifluoromethyl)phenyl)-1,1-dioxo-2-quinolin-4-ylmethyl-1,2-dihydro-1-benzo[d]isothiazol-3-one (5-(2,4-bis(trifluoromethyl)phenyl)-2-(quinolin-4-ylmethyl)saccharine) (compound 1.1)

### I.1.1 Preparation of 5-bromo-2-chlorosulfonyl-benzoic acid methyl ester

To a solution of 2-amino-5-bromo-benzoic acid methyl ester (25.0 g, 109 mmol) in aqueous hydrochloric acid (10 M, 100 ml) a solution of NaNO₂ (8.3 g, 119 mmol) in water (25 ml) was slowly added at 0° C. After stirring 1 hour at 0° C. the obtained reaction mixture was added to a saturated solution of SO₂ in 1,2-dichloroethane (75 ml) containing CuCl₂ (0.50 g, 3.7 mmol) and benzyltrimethmethylammonium chloride (1.41 g, 7.6 mmol) cooled to a temperature of 0° C. After the addition the reaction mixture was heated to 50° C. for 1 hour, cooled to ambient temperature and extracted with CH₂Cl₂ (250 ml). The organic phase was washed with an aqueous saturated solution of NaHCO₃, dried over Na₂SO₄, filtered and concentrated in vacuo. 5-Bromo-2-chlorosulfonyl-benzoic acid methyl ester was obtained as the residue in an amount of 3.6 g.

### I.1.2 Preparation of 5-bromo-2-(quinolin-4-ylmethyl)aminosulfonyl-benzoic acid methyl ester

A solution of quinolin-4-ylmethylamine (4.50 g, 24 mmol), crude 5-bromo-2-chlorosulfonyl-benzoic acid methyl ester (7.59 g, 24 mmol, prepared according to I.1.1) and triethylamine (4.04 ml, 29 mmol) in CH₂Cl₂ (100 ml) was stirred at ambient temperature for 10 hours. Water (100 ml) was added to the obtained reaction mixture. After separation of the phases, the organic phase was dried over Na₂SO₄, filtered and concentrated in vacuo. The residue was purified by column chromatography (gradient: cyclohexane/ethyl acetate; 100:1 to 1:9) to yield 5-bromo-2-(quinolin-4-ylmethyl-aminosulfonyl-benzoic acid methyl ester (2.48 g).

### I.1.3 Preparation of 5-(2,4-bis(trifluoromethyl)phenyl)-1,1-dioxo-2-quinolin-4-ylmethyl-1,2-dihydro-1-benzo[d]isothiazol-3-one

A solution of 5-bromo-2-(quinolin-4-ylmethyl)aminosulfonyl-benzoic acid methyl ester (0.50 g, 1.2 mmol, prepared according to I.1.2) and 2,4-bis(trifluoromethyl)phenylboronic acid (0.34 g, 1.3 mmol) in acetonitrile (5 ml) and water (2 ml) was refluxed for 8 hours in the presence of immobilized diisopropylethylamine (1.20 g, 4.08 mmol, PSDIPEA, from Novabiochem), tri-tert-butylphosphoniumtetrafluoroborat (0.017 g, 0.06 mmol) and bis(triphenylphosphino)palladium-II-chloride (0.025 g, 0.04 mmol). The obtained reaction mixture was filtered, the retained solid was rinsed with acetonitrile (10 ml) and the obtained filtrate was concentrated in vacuo. The residue was purified by column chromatography (gradient: cyclohexane/ethyl acetate; 90:10 to 40:60) to yield 5-(2,4-bis(trifluoromethyl)phenyl)-1,1-dioxo-2-quinolin-4-ylmethyl-1,2-dihydro-1-benzo[d]isothiazol-3-one (0.096 g).

### I.2 Preparation of 5-(2,4-difluorophenyl)-1,1-dioxo-2-quinolin-4-ylmethyl-1,2-dihydro-1-benzo[d]isothiazol-3-one (5-(2,4-difluorophenyl)-2-(quinolin-4-ylmethyl)saccharine) (compound 1.2)

### I.2.1 Preparation of 5-bromo-1,1-dioxo-2-quinolin-4-ylmethyl-1,2-dihydro-1-benzo[d]-isothiazol-3-one

A solution of 5-bromo-2-(quinolin-4-ylmethyl)aminosulfonyl-benzoic acid methyl ester (0.90 g, 2.1 mmol, prepared according to I.1.2) in glacial acetic acid (50 ml) was stirred for 10 hours at 100° C. The glacial acetic acid was removed in vacuo. The residue was dissolved in ethyl acetate, washed with a saturated solution of NaHCO₃, dried over NaSO₄, filtered and concentrated in vacuo. 5-Bromo-1,1-dioxo-2-quinolin-4-ylmethyl-1,2-dihydro-1-benzo[d]isothiazol-3-one was thus obtained in an amount of 0.70 g.

### I.2.2 Preparation of 5-(2,4-difluorophenyl)-1,1-dioxo-2-quinolin-4-ylmethyl-1,2-dihydro-1-benzo[d]isothiazol-3-one

A solution of 5-bromo-1,1-dioxo-2-quinolin-4-ylmethyl-1,2-dihydro-1-benzo[d]isothiazol-3-one (0.300 g, 0.7 mmol, prepared according to I.2.1) and 2,4-difluorophenyl-boronic acid (0.24 g, 2.4 mmol) in acetonitrile (5 ml) and water (2 ml) was refluxed for 3.5 hours in the presence of triethylamine (0.240 g, 2.4 mmol), tri-tert-butylphosphoniumtetrafluoroborat (0.010 g, 0.04 mmol) and bis(triphenylphosphino)palladium-II-chloride (0.015 g, 0.02 mmol). The reaction mixture was concentrated in vacuo. The residue was dissolved in glacial acetic acid and heated to 100° C. for 10 h. The reaction mixture was concentrated in vacuo. The residue was dissolved in ethyl acetate, washed with a saturated aqueous solution of NaHCO₃, dried over NaSO₄, filtered and concentrated in vacuo. The residue was purified by column chromatography (gradient: cyclohexane/ethyl acetate; 99:1 to 30:70) to yield 5-(2,4-difluorophenyl)-1,1-dioxo-2-quinolin-4-ylmethyl-1,2-dihydro-1-benzo[d]isothiazol-3-one (45 mg).

### I.3 Preparation of 5-(2,4-difluorophenyl)-2-(2-methoxypyridin-4-ylmethyl)-1,1-dioxo-1,2-dihydrobenzo[d]isothiazol-3-ylideneamine (compound 1.3)

### I.3.1 Preparation of 5-(2,4-difluorophenyl)benzo[d]isothiazole

A solution of 5-bromobenzo[d]isothiazole (5.0 g, 23 mmol, prepared according to EP 454621), 2,4-difluorophenylboronic acid (5.4 g, 34 mmol), tri-tert-butylphosphonium tetrafluoroborate (0.9 g), bis(triphenylphosphine)palladium(11)chloride (1.3 g) and triethylamine (14 ml) in a acetonitrile (40 ml) and water (20 ml) was heated at 75° C. for 2 h. The solvent was removed by distillation, water was added and the product was extracted with dichloromethane. The organic layer was dried over NaSO₄, filtered and concentrated in vacuo to yield crude 5-(2,4-difluorophenyl)benzo[d]isothiazole (5.8 g).

### I.3.2 Preparation of 2′,4′-difluoro-4-mercaptobiphenyl-3-ylcarbonitrile

To a solution of crude 5-(2,4-difluorophenyl)benzo[d]isothiazole (5.8 g, 23 mmol, prepared according to example I.3.1) in acetonitrile (100 ml) was added sodium methylate (2.9 g, 53 mmol) at room temperature. The mixture was heated to reflux for 2 hours then the solvent was removed by distillation, water was added and the mixture was acidified with aqueous HCl (10% strenght). The reaction mixture was extracted with dichloromethane and the organic layer was dried over NaSO₄, filtered and concentrated in vacuo. The residue was dissolved in methyl tert-butyl ether and sodium methylate (30% strenght in methanol) was added. The resulting solid precipitate was collected by filtration, washed with methyl tert-butyl ether and dried in vacuo. The dried solid precipitate was taken up in aqueous HCl (10% strenght) and extracted with dichloromethane. The organic layer was dried over NaSO₄, filtered and concentrated in vacuo to yield crude 2′,4′-difluoro-4-mercaptobiphenyl-3-ylcarbonitrile (4.5 g).

### I.3.3 Preparation of 3-cyano-2′,4′-difluorobiphenyl-4-sulfonyl chloride

Gaseous chlorine was passed through a solution of crude 2′,4′-difluoro-4-mercaptobiphenyl-3-ylcarbonitrile (4.5 g, 18 mmol, prepared according to example I.3.2) in acetic acid (45 ml) and water (3 ml) until the reaction was complete. Water was added and the product was extracted with dichloromethane. The organic layer was dried over NaSO₄, filtered and concentrated in vacuo. The residue was purified by column chromatography (eluent: toluene) to yield 3-cyano-2′,4′-difluorobiphenyl-4-sulfonyl chloride (4.6 g).

### I.3.4 3-Cyano-2′,4′-difluorobiphenyl-4-sulfonic acid N-(2-methoxypyridin-4-ylmethyl)amide

To a solution of (2-methoxypyridin-4-yl)-methylamine (705 mg, 5.1 mmol), pyridine (1.7 ml) and dimethyl amino pyridine (12 mg) in tetrahydrofuran (30 ml) was added a solution of 3-cyano-2′,4′-difluorobiphenyl-4-sulfonyl chloride (1.46 g, 4.6 mmol, prepared according to example I.3.3) in tetrahydrofuran (20 ml) at room temperature and the mixture was stirred for 48 hours. Water was added and the product was extracted with dichloromethane. The organic layer was washed with aqueous HCl (10% strenght) and an aqueous solution of sodium carbonate (10% strenght). The organic layer was dried over NaSO₄, filtered and concentrated in vacuo to yield 3-cyano-2′,4′-difluorobiphenyl-4-sulfonic acid N-(2-methoxy-pyridin-4-ylmethyl)amide (1.7 g).

### I.3.5 Preparation of 5-(2,4-difluorophenyl)-2-(2-methoxypyridin-4-ylmethyl)-1,1-dioxo-1,2-dihydro-benzo[d]isothiazol-3-ylideneamine

To a solution of 3-cyano-2′,4′-difluorobiphenyl-4-sulfonic acid N-(2-methoxypyridin-4-ylmethyl)amide (1.0 g, 2.4 mmol, prepared according to example I.3.4) in toluene (100 ml) was added a solution of sodium carbonate (0.77 g, 7.2 mmol) in water (50 ml). The mixture was stirred at room temperature for 24 hours. The organic phase was separated and the aqueous phase was extracted with ethyl acetate. The combined organic phases were dried over NaSO₄, filtered and concentrated in vacuo to yield 5-(2,4-difluoro-phenyl)-2-(2-methoxypyridin-4-ylmethyl)-1,1-dioxo-1,2-dihydro-benzo[d]isothiazol-3-ylideneamine (0.9 g; m.p.=173° C.; HPLC/MS: t₁=2.9 min, (m/z)=416[M+H]⁺)

### I.4 Preparation of 5-(2,4-difluorophenyl)-2-(2-methoxypyridin-4-ylmethyl)-1,1-dioxo-1,2-dihydro-benzo[d]isothiazol-3-one (compound 1.4)

To a solution of 5-(2,4-difluorophenyl)-2-(2-methoxypyridin-4-ylmethyl)-1,1-dioxo-1,2-dihydrobenzo[d]isothiazol-3-ylideneamine (0.6 g, 1.4 mmol, prepared according to example I.3.5) in dioxane (30 ml) was added aqueous HCl (10% strenght, 5 ml). The solution was stirred for 5 hours at room temperature. Water was added and the product was extracted with dichloromethane. The organic layer was dried over NaSO₄, filtered and concentrated in vacuo. The residue was purified by column chromatography (eluent: cyclohexane/ethyl acetate) to yield 5-(2,4-difluorophenyl)-2-(2-methoxypyridin-4-ylmethyl)-1,1-dioxo-1,2-dihydro-benzo[d]isothiazol-3-one (0.73 mmol, 300 mg).

¹H-NMR (CDCl₃): δ=3.92 (s, 3H), 4.86 (s, 2H), 6.84 (s, 1H), 7.01 (m, 3H), 7.44 (m, 1H), 8.02 (s, 2H), 8.18 ppm (m, 2H).

Sulfonamide compounds of formula (I.A), i.e. compounds of formulas (I) wherein m is 0, X is O, Y is CH and Z is a chemical bond,

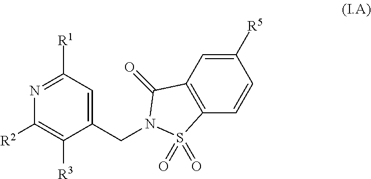

wherein R¹, R², R³ and R⁵ have the meanings given in table I below, have been prepared according to the methods outlined before (compounds I.5 to I.48).

TABLE I

| | | | | | HPLC-MS; |
|---|---|---|---|---|---|
| | R¹ | R² | R³ | R⁵ | mp |
| I.5 | H | —CH═CH—CH═CH— | | 4-chlorophenyl | |
| I.6 | H | —CH═CH—CH═CH— | | 4-(trifluoromethyl)-phenyl | |
| I.7 | H | —CH═CH—CH═CH— | | 4-cyanophenyl | |
| I.8 | H | —CH═CH—CH═CH— | | 4-(trifluoromethoxy)-phenyl | |
| I.9 | H | —CH═CH—CH═CH— | | 2,4-dichlorophenyl | |
| I.10 | H | —CH═CH—CH═CH— | | 2,4-di(trifluoromethyl)-phenyl | RT = 3.229 min, |
| | | | | | M_{w} = 468.60 g/mol |
| I.11 | H | —CH═CH—CH═CH— | | 2-chloro-4-(trifluoro-methyl)phenyl | |
| I.12 | H | —CH═CH—CH═CH— | | 2-(trifluoromethyl)-phenyl | |
| I.13 | H | —CH═CH—CH═CH— | | 2-chlorophenyl | |
| I.14 | H | —CH═CH—CH═CH— | | 4-chloro-2-(trifluoro-methyl)phenyl | |
| I.15 | H | —CH═CH—CH═CH— | | 2,4-difluorophenyl | |
| I.16 | H | —CH═CH—CH═CH— | | 2-fluorophenyl | |
| I.17 | H | H | H | 2,4-dichlorophenyl | RT = 2.813 min, |
| | | | | | M_{w} = 417.95 g/mol; 151.5° C. |
| I.18 | OCH₃ | H | H | 2,4-dichlorophenyl | RT = 3.855 min, |
| | | | | | M_{w} = 449.05 g/mol; 139.5° C. |
| I.19 | OCH₃ | H | H | 2,4-difluorophenyl | RT = 3.680 min, |
| | | | | | M_{w} = 417.10 g/mol |
| I.20 | H | H | H | 2,4-difluorophenyl | RT = 2.587 min, |
| | | | | | M_{w} = 387.05 g/mol |
| I.21 | OCH₃ | H | H | 4-(trifluoromethyl)-phenyl | RT = 3.896 min, |
| | | | | | M_{w} = 517.05 g/mol |
| I.22 | H | CH₃ | CH₃ | 4-(trifluoromethyl)-phenyl | RT = 3.271 min, |
| | | | | | M_{w} = 515.05 g/mol |
| I.23 | H | CH₃ | CH₃ | 2,4-difluorophenyl | RT = 2.837 min, |
| | | | | | M_{w} = 415.05 g/mol |
| I.24 | H | CH₃ | CH₃ | 2,4-dichlorophenyl | RT = 3.013 min, |
| | | | | | M_{w} = 446.60 g/mol |
| I.25 | H | H | CH₃ | 2,4-difluorophenyl | RT = 2.605 min, |
| | | | | | M_{w} = 400.70 g/mol |
| I.26 | H | H | CH₃ | 2,4-di(trifluoromethyl)-phenyl | RT = 3.237 min, |
| | | | | | M_{w} = 501.05 g/mol |
| I.27 | H | —CH═C(OCH₃)—CH═CH— | | 2,4-difluorophenyl | RT = 2.892 min, |
| | | | | | M_{w} = 467.05 g/mol |
| I.28 | H | —CH═C(CF₃)—CH═CH— | | 2,4-difluorophenyl | RT = 3.809 min, |
| | | | | | M_{w} = 504.60 g/mol |
| I.29 | H | H | F | 2,4-dichlorophenyl | RT = 3.650 min, |
| | | | | | M_{w} = 437.05 g/mol; 180° C. |
| I.30 | H | —CH═C(CF₃)—CH═CH— | | 2,4-dichlorophenyl | RT = 4.108 min, |
| | | | | | M_{w} = 536.50 g/mol; 224° C. |
| I.31 | H | —CH═CF—CH═CH— | | 2,4-dichlorophenyl | RT = 3.733 min, |
| | | | | | M_{w} = 487.00 g/mol |
| I.32 | H | H | CH₃ | 2,4-dichlorophenyl | RT = 3.268 min, |
| | | | | | M_{w} = 433.00 g/mol; 197° C. |
| I.33 | H | H | H | 2-chloro-4- | RT = 2.951 min, |
| | | | | (trifluoromethyl)phenyl | M_{w} = 452.60 g/mol; 179° C. |
| I.34 | H | CH₃ | CH₃ | 2-chloro-4- | RT = 3.102 min, |
| | | | | (trifluoromethyl)phenyl | M_{w} = 481.15 g/mol; 170° C. |
| I.35 | H | CH₃ | CH₃ | 4-chloro-2- | RT = 3.061 min, |
| | | | | (trifluoromethyl)phenyl | M_{w} = 481.15 g/mol |
| I.36 | H | H | H | 2,3,4-trifluorophenyl | RT = 2.643 min, |
| | | | | | M_{w} = 405.15 g/mol; 165° C. |
| I.37 | OCH₃ | H | H | 2,3,4-trifluorophenyl | RT = 3.373 min, |
| | | | | | M_{w} = 434.60 g/mol |
| I.38 | H | CH₃ | CH₃ | 2,3,4-trifluorophenyl | RT = 2.741 min, |
| | | | | | M_{w} = 433.15 g/mol; 143° C. |
| I.39 | H | —CH═C(OCH₃)—CH═CH— | | 2,4-dichlorophenyl | RT = 3.220 min, |
| | | | | | M_{w} = 498.50 g/mol |
| I.40 | OCH₃ | H | H | 2-chloro-4-(trifluormethyl)phenyl | RT = 3.738 min, |
| | | | | | M_{w} = 482.60 g/mol |
| I.41 | H | H | H | 2-chloro-4-cyanophenyl | RT = 2.630 min, |
| | | | | | M_{w} = 410.15 g/mol |
| I.42 | OCH₃ | H | H | 2-chloro-4-cyanophenyl | RT = 3.542 min, |
| | | | | | M_{w} = 440.15 g/mol |
| I.43 | H | CH₃ | CH₃ | 4-cyano-2-(trifluoromethyl)phenyl | RT = 2.856 min, |
| | | | | | M_{w} = 472.15 g/mol; 205° C. |
| I.44 | H | —CH═CH—CH═CH— | | 2,3,4-trifluorophenyl | RT = 3.129 min, |
| | | | | | M_{w} = 455.2 g/mol |
| I.45 | H | H | H | 3-chloro-5-(trifluoromethyl)- | RT = 2.818 min, |
| | | | | 2-pyridyl | M_{w} = 454.2 g/mol |
| I.46 | OCH₃ | H | H | 3-chloro-5-(trifluoromethyl)- | RT = 3.808 min, |
| | | | | 2-pyridyl | M_{w} = 483.2 g/mol |
| I.47 | H | CH₃ | CH₃ | 3-chloro-5-(trifluoromethyl)- | RT = 2.900 min, |
| | | | | 2-pyridyl | M_{w} = 481.6 g/mol |

| | | | | |
|---|---|---|---|---|
| I.48 | H | —CH═CH—CH═CH— | 2-chloro-4-cyanophenyl I | RT = 2.852 min, |
| | | | | M_{w} = 459.6 g/mol |

mp = melting point
RT = retention time
M_{w} = molecular weight

### II. Activity Against Insects and Arachnids

### General Conditions

If not otherwise specified, test solutions were prepared as follow: The active compound is dissolved at the desired concentration in a mixture of 1:1 (vol/vol) distilled water:aceton. The test solution is prepared at the day of use. Test solutions are prepared in general at concentrations of 1000, 500, 300, 100 and 30 ppm (wt/vol).

II.1 Boll Weevil (anthonomus grandis)

For evaluating control of boll weevil (Anthonomus grandis) the test unit consisted of 24-well-microtiter plates containing an insect diet and 20-30 anthonomus grandis eggs. The compounds were formulated using a solution containing 75% (vol/vol) water and 25% (vol/vol) DMSO. Different concentrations of formulated compounds were sprayed onto the insect diet at 20 μl, using a custom built micro atomizer, at two replications. After application, microtiter plates were incubated at about 23 (±1)° C. and about 50 (±5) % relative humidity for 5 days. Egg and larval mortality was then visually assessed.

In this test, compounds I.9, I.12, I.17, I.18, I.25-I.28, I.33-I.35 and I.38 at 2500 ppm showed over 75% mortality in comparison with untreated controls.

II.2 Mediterranean Fruitfly (ceratitis capitata)

For evaluating control of Mediterranean fruitfly (Ceratitis capitata) the test unit consisted of microtiter plates containing an insect diet and 50-80 Ceratitis capitata eggs. The compounds were formulated using a solution containing 75% (v:v) water and 25% (v:v) DMSO. Different concentrations of formulated compounds were sprayed onto the insect diet at 5 μl, using a custom built micro atomizer, at two replications. After application, microtiter plates were incubated at about 28 (±1)° C. and about 80 (±5) % relative humidity for 5 days. Egg and larval mortality was then visually assessed.

In this test, compounds I.9, I.12, I.17, I.25, I.27, I.33-I.35 and I.38 at 2500 ppm showed over 75% mortality in comparison with untreated controls.

II.3 Tobacco Budworm (heliothis virescens)

For evaluating control of tobacco budworm (Heliothis virescens) the test unit consisted of 96-well-microtiter plates containing an insect diet and 15-25 Heliothis virescens eggs. The compounds were formulated using a solution containing 75% (vol/vol) water and 25% (vol/vol) DMSO. Different concentrations of formulated compounds were sprayed onto the insect diet at 10 μl, using a custom built micro atomizer, at two replications. After application, microtiter plates were incubated at about 28 (±1)° C. and about 80 (±5) % relative humidity for 5 days. Egg and larval mortality was then visually assessed.

In this test, compounds I.9, I.12, I.17, I.18, I.26, I.28, I.33-I.35 and I.38 at 2500 ppm showed over 75% mortality in comparison with untreated controls.

II.4 Vetch Aphid (megoura viciae)

For evaluating control of vetch aphid (Megoura viciae) through contact or systemic means the test unit consisted of 24-well-microtiter plates containing broad bean leaf disks. The compounds were formulated using a solution containing 75% (vol/vol) water and 25% (vol/vol) DMSO. Different concentrations of formulated compounds were sprayed onto the leaf disks at 2.5 μl, using a custom built micro atomizer, at two replications. After application, the leaf disks were air-dried and 5-8 adult aphids placed on the leaf disks inside the microtiter plate wells. The aphids were then allowed to suck on the treated leaf disks and incubated at about 23 (±1° C. and about 50 (±5% relative humidity for 5 days. Aphid mortality and fecundity was then visually assessed.

In this test, compounds I.9, I.12, I.17, I.18, I.25, I.27, I.34, I.35 and I.38 at 2500 ppm showed over 75% mortality in comparison with untreated controls.

II.5 Green Peach Aphid (myzus persicae)

For evaluating control of green peach aphid (Myzus persicae) through systemic means the test unit consisted of 96-well-microtiter plates containing liquid artificial diet under an artificial membrane. The compounds were formulated using a solution containing 75% (vol/vol) water and 25% (vol/vol) DMSO. Different concentrations of formulated compounds were pipetted into the aphid diet, using a custom built pipetter, at two replications. After application, 5-8 adult aphids were placed on the artificial membrane inside the microtiter plate wells. The aphids were then allowed to suck on the treated aphid diet and incubated at about 23 (±1)° C. and about 50 (±5) % relative humidity for 3 days. Aphid mortality and fecundity was then visually assessed.

In this test, compounds I.9, I.12, I.17, I.18, I.26, I.27, I.34, I.35 and I.38 at 2500 ppm showed over 75% mortality in comparison with untreated controls

II.6 Orchid Thrips (dichromothrips corbetti)

Dichromothrips corbetti adults used for bioassay were obtained from a colony maintained continuously under laboratory conditions. For testing purposes, the test compound was diluted to a concentration of 300 ppm (wt compound: vol diluent) in a 1:1 mixture of acetone:water (vol/vol), plus 0.01% (vol/vol) Kinetic® surfactant.

Thrips potency of each compound was evaluated by using a floral-immersion technique. Plastic petri dishes were used as test arenas. All petals of individual, intact orchid flowers were dipped into treatment solution and allowed to dry. Treated flowers were placed into individual petri dishes along with 10-15 adult thrips. The petri dishes were then covered with lids. All test arenas were held under continuous light and a temperature of about 28° C. for duration of the assay. After 4 days, the numbers of live thrips were counted on each flower, and along inner walls of each petri dish. The level of thrips mortality was extrapolated from pre-treatment thrips numbers.

In this test, compounds I.8-I.13, I.15, I.19, I.20, I.22, I.23, I.27 and I.31 at 300 ppm showed over 75% mortality in comparison with untreated controls.

II.7 Cowpea Aphid (aphis craccivora)

The active compound is dissolved at the desired concentration in a mixture of 1:1 (vol/vol) distilled water:acetone. The test solution is prepared at the day of use.

Potted cowpea plants colonized with approximately 100-150 aphids of various stages were sprayed after the pest population has been recorded. Population reduction was assessed after 24, 72, and 120 hours.

In this test, compounds I.8-I.13, I.15, I.17-I.20, I.22-I.27, I.29 and I.31 at 300 ppm showed over 75% mortality in comparison with untreated controls.

II.8 Cotton Aphid (aphis gossypii) I

The active compounds were formulated in 50:50 (vol/vol) acetone:water and 100 ppm Kinetic™ surfactant.

Cotton plants at the cotyledon stage (one plant per pot) were infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.

In this test, compounds I.5-I.20, I.22-I.25, I.28 and I.31 at 300 ppm showed over 75% mortality in comparison with untreated controls.

II.9 Silverleaf Whitefly (bemisia argentifolii)

The active compounds were formulated in 50:50 acetone:water (vol/vol) and 100 ppm Kinetic™ surfactant.

Selected cotton plants were grown to the cotyledon state (one plant per pot). The cotyledons were dipped into the test solution to provide complete coverage of the foliage and placed in a well-vented area to dry. Each pot with treated seedling was placed in a plastic cup and 10 to 12 whitefly adults (approximately 3-5 day old) were introduced. The insects were collected using an aspirator and a Tygon® tubing connected to a barrier pipette tip. The tip containing the collected insects was then gently inserted into the soil containing the treated plant allowing insects to crawl out of the tip to reach the foliage for feeding. The cups were covered with a re-usable screened lid. Test plants were maintained in the holding room at about 25° C. and about 20-40% relative humidity for 3 days avoiding direct exposure to the fluorescent light (24 hour photoperiod) to prevent trapping of heat inside the cup. Mortality was assessed 3 days after treatment of the plants.

In this test, compounds I.22-I.24 and I.28 at 300 ppm showed over 75% mortality in comparison with untreated controls.

II.10 Colorado Potato Beetle (leptinotarsa decemlineata)

Potato plants are utilized for bioassays. Excised plant leaves are dipped into 1:1 (vol/vol) acetone/water dilutions of the active compounds. After the leaves have dried, they are individually placed onto water-moistened filter paper on the bottoms of Petri dishes. Each dish is infested with 5-7 larvae and covered with a lid. Each treatment dilution is replicated 4 times. Test dishes are held at approximately 27° C. and about 60% humidity. Numbers of live and morbid larvae are assessed in each dish at 5 days after treatment application, and percent mortality is calculated.

In this test, compounds I.6-I.8 at 300 ppm showed over 75% mortality in comparison with untreated controls.

II.11 Green Peach Aphid (myzus persicae)

The active compounds were formulated in 50:50 acetone:water (vol/vol) and 100 ppm Kinetic™ surfactant.

Pepper plants in the 2nd leaf-pair stage (variety “California Wonder”) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and about 20-40% relative humidity. Aphid mortality on the treated plants, relative to mortality on check plants, was determined after 5 days.

In this test, compounds I.8-I.17, I.20 and I.22-I.24 at 300 ppm showed over 75% mortality in comparison with untreated controls.

II.12 Rice Green Leafhopper (nephotettix virescens)

Rice seedlings were cleaned and washed 24 hours before spraying. The active compounds were formulated in 50:50 acetone:water (vol/vol), and 0.1% (vol/vol) surfactant (EL 620) was added. Potted rice seedlings were sprayed with 5 ml test solution, air dried, placed in cages and inoculated with 10 adults. Treated rice plants were kept at about 28-29° C. and relative humidity of about 50-60%. Percent mortality was recorded after 72 hours.

In this test, compounds I.19 and I.22-I.24 at 300 ppm showed over 75% mortality in comparison with untreated controls.

II.13 Rice Brown Plant Hopper (nilaparvata lugens)

Rice seedlings were cleaned and washed 24 hours before spraying. The active compounds were formulated in 50:50 acetone:water (vol/vol) and 0.1% vol/vol surfactant (EL 620) was added. Potted rice seedlings were sprayed with 5 ml test solution, air dried, placed in cages and inoculated with 10 adults. Treated rice plants were kept at about 28-29° C. and relative humidity of about 50-60%. Percent mortality was recorded after 72 hours.

In this test, compounds I.9, I.11, I.13, I.15, I.22 and I.23 at 300 ppm showed over 75% mortality in comparison with untreated controls.

II.14 Diamond Back Moth (plutella xylostella)

The active compound is dissolved at the desired concentration in a mixture of 1:1 (vol/vol) distilled water:acteon. The test solution is prepared at the day of use. Leaves of Chinese cabbage are dipped in test solution and air-dried. Treated leaves are placed in petri dished lined with moist filter paper. Mortality is recorded 24, 72, and 120 hours after treatment. Behavior of treated larvae, e.g. hypoactivity and hyperactivity, presence of pupal cocoon, as well as characteristics of dead larvae is also be noted.

In this test, compounds I.8-I.13, I.15, I.19, I.20, I.22-I-24, I.27 and I.31 at 300 ppm showed over 75% mortality in comparison with untreated controls.

II.15 Southern Armyworm (spodoptera eridania, 2^{nd} instar larvae)

The active compounds were formulated in 50:50 acetone:water (vol/vol) and 100 ppm Kinetic™ surfactant.

A pair of first true leaves of Sieva lima bean was dipped into the test solution and allowed to dry. The leaves were then placed in a plastic perforated zip enclosure bag and ten 2^{nd} instar larvae were added. At 4 days, observations were made of mortality and reduced feeding.

In this test, compounds I.6-I.16, I.19, I.21-I.24 and I.31 at 300 ppm showed over 75% mortality in comparison with untreated controls.

II.16 Spider Mite (tetranychus spp.)

The active compound is dissolved at the desired concentration in a mixture of 1:1 (vol/vol) distilled water:acetone. The test solution is prepared at the day of use.

Potted cotton plants colonized with approximately 50 mites of various stages are sprayed after the pest population has been recorded. Population reduction (or increase) after 24, 72, and 120 hours is assessed.

In this test, compounds I.8, I.10, I.11, I.15, I.19, I.23 and I.24 at 300 ppm showed over 75% mortality in comparison with untreated controls.

### III. Activity Against Rodents

Some representatives of the compounds according to the present invention have been tested in acute oral rat toxicity studies.

In these studies, test substances were administered to adult female rats by oral gavage for one time. The compounds were administered as a 1% b.w. suspension or the compound in a 1% aqueous carboxymethyl cellulose solution. In each study three rats were used. The animals were treated with a single dose of 100 mg/kg bw. The animals were observed for clinical signs of toxicity including death for 14 days post-administration in order to identify possible delayed toxicity. After the 14 day observation period, the animals were sacrificed, dissected, and examined for gross pathological lesions. The results are summarized in table II:

TABLE II

| | Lethality (cumulative | | | | |
|---|---|---|---|---|---|
| Compound* | 0-5 h* | 1 d* | 2 d* | 7 d* | 14 d* |
| I.34 | 0 | 0 | 0 | 0 | 1 |
| I.35 | 0 | 0 | 0 | 0 | 2 |

*time after treatment
**according to table I

Remarkably, these compounds differed in their induction of clinical signs of toxicity, however, no such signs were observed directly or within the first 24 h or more after treatment. This delayed toxicity after single oral dosing accompanied by a lack of immediate clinical symptoms renders these compounds suitable for as rodenticides.

1-29. (canceled)

30. A compound of formula (I) 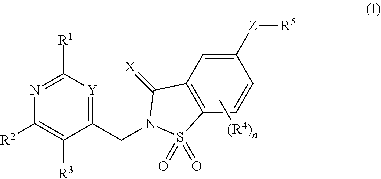
wherein
R¹ is selected from the group consisting of hydrogen, halogen, cyano, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio,
C₂-C₆-alkynylthio, C₁-C₆-haloalkyl and C₁-C₆-haloalkoxy;
R² and R³ are independently selected from the group consisting of hydrogen, halogen, cyano, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy; or
R² together with R³ and together with the carbon atoms to which they are bound form a fused 5 or 6-membered carbocycle or a fused 5- or 6-membered heterocycle, which contains 1 or 2 heteroatoms selected from the group consisting of O, N and S as ring members, wherein each fused carbocycle or heterocycle is unsubstituted or carries 1, 2, 3 or 4 substituents, independently of one another selected from the group consisting of halogen, cyano, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy;
R⁴ is selected from the group consisting of halogen, cyano, C₁-C₆-alkyl, C₂-C₆-alkenyl, C₂-C₆-alkynyl, C₁-C₆-alkoxy, C₁-C₆-alkylthio, C₁-C₆-alkylsulfinyl, C₁-C₆-alkylsulfonyl,
C₁-C₆-haloalkyl, C₁-C₆-haloalkoxy, C₁-C₆-haloalkylthio, C₁-C₆-haloalkylsulfinyl and C₁-C₆-haloalksulfonyl;
n is 0, 1, 2 or 3;
R⁵ is selected from the group consisting of phenyl and a 5- or 6-membered heterocycle Het, which contains 1, 2 or 3 heteroatoms selected from the group consisting of O, N and S as ring members, wherein phenyl and Het are unsubstituted or carry 1, 2, 3 or 4 substituents, independently of one another selected from the group consisting of halogen, cyano, C₁-C₆-alkyl, C₂-C₆-alkenyl, C₂-C₆-alkynyl, C₃-C₇-cycloalkyl, C₁-C₆-alkoxy, C₁-C₆-alkylthio, C₁-C₆-alkylsulfinyl, C₁-C₆-alkylsulfonyl, C₁-C₆-haloalkyl, C₁-C₆-haloalkoxy C₁-C₆-haloalkylthio, C₁-C₆-haloalkylsulfinyl and C₁-C₆-haloalkylsulfonyl;
X is O or NR^{x}, wherein R^{x} is selected from the group consisting of hydrogen, C₁-C₆-alkyl, C₂-C₆-alkenyl, C₂-C₆-alkynyl, C₃-C₇-cycloalkyl, C₁-C₆-alkoxy, C₂-C₆-alkenyloxy, C₁-C₆-alkylcarbonyl and C₁-C₆-alkylcarbonyloxy;
Y is N or C(R^{y}), wherein R^{y} is selected from the group consisting of hydrogen, halogen, cyano, C₁-C₆-alkyl, C₂-C₆-alkenyl, C₂-C₆-alkynyl, C₃-C₇-cycloalkyl, C₃₋₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy; and
Z is a chemical bond, O or N(R^{z}), wherein R^{z} is selected from the group consisting of C₁-C₆-alkyl, C₂-C₆-alkenyl, C₂-C₆-alkynyl, C₃-C₇-cycloalkyl, C₁-C₆-alkoxy, C₂-C₆-alkenyloxy, C₁-C₆-alkylcarbonyl and C₁-C₆-alkylcarbonyloxy;
and the N-oxides and salts thereof.

31. The compound according to claim 30, wherein R¹ is selected from the group consisting of hydrogen, halogen, C₁-C₆-alkyl, C₃-C₇-cycloalkyl, C₁-C₆-alkoxy, C₁-C₆-alkylthio, C₁-C₆-haloalkyl and C₁-C₆-haloalkoxy.

32. The compound of claim 30, wherein R² and R³ are independently selected from the group consisting of hydrogen, halogen, C₁-C₆-alkyl, C₃-C₇-cycloalkyl, C₁-C₆-alkoxy, C₁-C₆-alkylthio, C₁-C₆-haloalkyl and C₁-C₆-haloalkoxy.

33. The compound of claim 30, wherein R² together with R³ and together with the carbon atoms to which they are bound form a fused 5 or 6-membered carbocycle or a fused 5- or 6-membered heterocycle, which contains 1 heteroatom selected from the group consisting of O, N and S as a ring member, wherein each fused carbocycle or heterocycle is unsubstituted or carries 1 or 2 substituents, independently of one another selected from the group consisting of halogen, cyano, C₁-C₆-alkyl, C₃-C₇-cycloalkyl, C₁-C₆-alkoxy, C₁-C₆-alkylthio, C₁-C₆-haloalkyl and C₁-C₆-haloalkoxy.

34. The compound of claim 30, wherein Y is C(R^{y}) and R^{y} is selected from the group consisting of hydrogen, halogen, C₁-C₆-alkyl, C₃-C₇-cycloalkyl, C₁-C₆-alkoxy, C₁-C₆-alkylthio, C₁-C₆-haloalkyl and C₁-C₆-haloalkoxy.

35. The compound of claim 30, wherein X is O or NH.

36. The compound of claim 30, wherein R⁴ is selected from halogen, C₁-C₄-alkyl and C₁-C₄-haloalkyl.

37. The compound of claim 30, wherein n is 0.

38. The compound of claim 30, wherein Z is a chemical bond or O.

39. The compound of claim 30, wherein R⁵ is selected from the group consisting of phenyl which is unsubstituted or carries 1 or 2 substituents, independently of one another selected from halogen, C₁-C₄-alkyl, C₃-C₇-cycloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₁-C₄-haloalkylsulfinyl and C₁-C₄-haloalkylsulfonyl.

40. The compound of claim 39, wherein Z is a chemical bond.

41. The compound of claim 39, wherein R⁵ is a phenyl which carries 1 substituent in the 2-position or in the 4-position relative to the bonding position or a phenyl which carries 2 substituents in the 2- and in the 4-position or in the 2- and in the 5-position relative to the bonding position.

42. The compound of claim 38, wherein R⁵ is selected from 5- or 6-membered heterocycles Het, which contain 1, 2 or 3 heteroatoms selected from the group consisting of O, N and S as ring members, wherein Het is unsubstituted or carries 1 or 2 substituents, independently of one another selected from halogen, C₁-C₄-alkyl, C₃-C₇-cycloalkyl, C₁-C₄-alkoxy, C₁-C₄-alkylsulfonyl, C₁-C₄-haloalkyl, C₁-C₄-haloalkoxy, C₁-C₄-haloalkylthio, C₁-C₄-haloalkylsulfinyl and C₁-C₄-haloalkylsulfonyl.

43. The compound of claim 42, wherein Het is selected from the group consisting of furyl, pyrrolyl, thienyl, oxazolyl, isoxazolyl, pyrazolyl, imidazolyl, thiazolyl, isothiazolyl, 1,2,4-oxadiazolyl, 1,3,4-oxadiazolyl, 1,2,4-thiadiazolyl, 1,3,4-thiadiazolyl, 1,2,3-triazolyl, 1,2,4-triazolyl, pyridinyl, pyrazinyl, pyrimidinyl and pyridazinyl, wherein each of the aforementioned radicals are unsubstituted or carry 1 or 2 substituents, independently of one another selected from the group consisting of halogen, C₁-C₄-alkyl, C₃-C₇-cycloalkyl, C₁-C₄-alkoxy, C₁-C₄-alkylthio, C₁-C₄-alkylsulfinyl, C₁-C₄-alkylsulfonyl, C₁-C₄-haloalkyl, C₁-C₄-haloalkoxy, haloalkylthio, C₁-C₄-haloalkylsulfinyl and C₁-C₄-haloalkylsulfonyl.

44. The compound of claim 42, wherein Z is a O.

45. A process for the preparation the compound of claim 30 comprising, reacting a compound of formula (II), 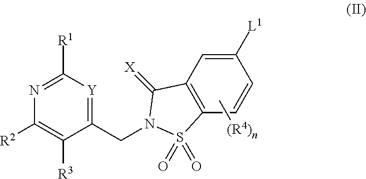
with a boronic acid derivative of the formula R⁵—(Z)—B(OR^{b1})(OR^{b2}) in the presence of a base and a transition metal catalyst to give a compound of formula (I),
wherein L¹ is a suitable leaving group and R^{b1} and R^{b2} are each independently hydrogen or C₁-C₄-alkyl, or R^{b1} and R^{b2} together form an 1,2-ethylene moiety the carbon atoms of which may be unsubstituted or may all or in part be substituted by methyl groups.

46. A process for preparing a compound of claim 30, comprising, submitting a compound of formula (VII), 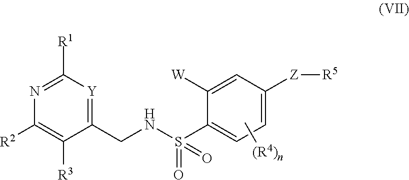
wherein W is CN or —C(═O)L², and L² is a suitable leaving group,
to an intramolecular cyclisation to give a compound of formula (I).

47. A process for preparing a compound of claim 30, comprising, reacting a pyridine or pyrimidine compound of formula (VIII) 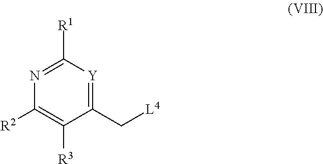
with a saccharine or iminosaccharine derivative of formula (IX), 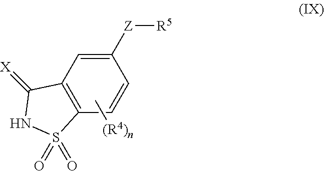
wherein L⁴ is a suitable leaving group.

48. A compound of formula (II), 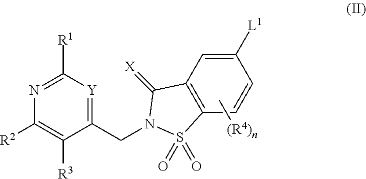
wherein
R¹ is selected from the group consisting of hydrogen, halogen, cyano, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio,
C₂-C₆-alkynylthio, C₁-C₆-haloalkyl and C₁-C₆-haloalkoxy;
R² and R³ are independently selected from the group consisting of hydrogen, halogen, cyano, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy; or
R² together with R³ and together with the carbon atoms to which they are bound form a fused 5 or 6-membered carbocycle or a fused 5- or 6-membered heterocycle, which contains 1 or 2 heteroatoms selected from the group consisting of O, N and S as ring members, wherein each fused carbocycle or heterocycle is unsubstituted or carries 1, 2, 3 or 4 substituents, independently of one another selected from the group consisting of halogen, cyano, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy;
R⁴ is selected from the group consisting of halogen, cyano, C₁-C₆-alkyl, C₂-C₆-alkenyl, C₂-C₆-alkynyl, C₁-C₆-alkoxy, C₁-C₆-alkylthio, C₁-C₆-alkylsulfinyl, C₁-C₆-alkylsulfonyl,
C₁-C₆-haloalkyl, C₁-C₆-haloalkoxy, C₁-C₆-haloalkylthio, C₁-C₆-haloalkylsulfinyl and C₁-C₆-haloalksulfonyl;
n is 0, 1, 2 or 3;
X is O or NR^{x}, wherein R^{x} is selected from the group consisting of hydrogen, C₁-C₆-alkyl, C₂-C₆-alkenyl, C₂-C₆-alkynyl, C₃-C₇-cycloalkyl, C₁-C₆-alkoxy, C₂-C₆-alkenyloxy, C₁-C₆-alkylcarbonyl and C₁-C₆-alkylcarbonyloxy;
Y is N or C(R^{y}), wherein R^{y} is selected from the group consisting of hydrogen, halogen, cyano, C₁-C₆-alkyl, C₂-C₆-alkenyl, C₂-C₆-alkynyl, C₃-C₇-cycloalkyl, C₃₋₇-cycloalkyl-C₁-C₄-alkyl, C₁-C₆-alkoxy, C₂-C₆-alkenyloxy, C₂-C₆-alkynyloxy, C₁-C₆-alkylthio, C₂-C₆-alkenylthio, C₂-C₆-alkynylthio, C₁-C₆-haloalkyl and C₁-C₆-haloalkoxy;
and
L¹ is halogen.

49. A composition containing at least one compound of claim 30 and at least one liquid or solid carrier.

50. A method for controlling animal pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from pest attack or infestation with a compound of claim 30 or a composition comprising the compound and at least one liquid or solid carrier.

51. A method for protecting plant propagation material and/or the plants which grow therefrom, which method comprises treating the plant propagation material with a compound of claim 30 or a composition comprising the compound and at least one liquid or solid carrier.

52. The method as claimed in claim 50, where the animal pests are selected from invertebrate pests.

53. The method as claimed in claim 50, where the animal pests are selected from rodent pests.

54. Plant propagation material, treated with a compound of claim 30 or a composition comprising the compound and at least one liquid or solid carrier.

55. A method for treating or protecting an animal from infestation or infection by parasites which comprises administering or applying to the animal or to their habitat a compound of claim 30 or a composition comprising the compound and at least one liquid or solid carrier.
